(19) United States
(12) Patent Application Publication
(10) Pub. No.: US 2025/0188153 A1
(43) Pub. Date: Jun. 12, 2025

(54) HMPV ANTIBODIES AND THEIR USE

(71) Applicant: University of Georgia Research Foundation, Inc., Athens, GA (US)

(72) Inventors: Jarrod Mousa, Athens, GA (US); Jiachen Huang, Athens, GA (US); Avik Banerjee, San Jose, CA (US)

(73) Assignee: University of Georgia Research Foundation, Inc., Athens, GA (US)

(21) Appl. No.: 18/844,829

(22) PCT Filed: Mar. 6, 2023
(86) PCT No.: PCT/US2023/063801

§ 371 Date: Sep. 6, 2024

Related U.S. Application Data
(60) Provisional application No. 63/317,384, filed on Mar. 7, 2022.

(51) Int. Cl. C07K 16/10; A61K 39/00; A61P 31/14; G01N 33/569

(52) U.S. Cl. CPC C07K 16/10; A61P 31/14; G01N 33/56983; A61K 2039/505; C07K 2317/33; C07K 2317/34; C07K 2317/73; C07K 2317/76; C07K 2317/92; G01N 2333/08

(57) ABSTRACT

Antibodies and antigen binding fragments that specifically bind to human metapneumovirus (hMPV) F protein and neutralize hMPV are disclosed. Nucleic acids encoding these antibodies, vectors and host cells are also provided. The disclosed antibodies, antigen binding fragments, nucleic acids and vectors can be used, for example, to inhibit an hMPV infection or detect a hMPV infection.

### CROSS REFERENCE TO RELATED APPLICATIONS

This claims the benefit of U.S. Provisional Application No. 63/317,384, filed Mar. 7, 2022, which is incorporated herein by reference.

### STATEMENT OF GOVERNMENT SUPPORT

This invention was made with Government support under grant number R01 AI143865 and 1K01 OD026569 from the National Institutes of Health. The United States Government has certain rights in the invention.

### FIELD OF THE DISCLOSURE

This related to the field of metapneumoviruses, specifically to monoclonal antibodies and antigen binding fragments thereof that specifically bind the human metapneumovirus F protein.

### REFERENCE TO AN ELECTRONIC SEQUENCE LISTING

The contents of the electronic sequence listing (8618-108103-02 SEQUENCE LISTING.xml; Size: 122,880 bytes; and Date of Creation: Feb. 20, 2023) is herein incorporated by reference in its entirety.

### BACKGROUND

Human metapneumovirus (hMPV) is a leading cause of respiratory disease in children and the elderly (Panda, et al., Int. J. Infect. Dis. 25, 45-52 (2014); Falsey et al., J. Infect. Dis. 187, 785-790 (2003); van den Hoogen et al., J. Infect. Dis. 188, 1571-1577 (2003); Madhi et al., Clin. Infect. Dis. 37, 1705-1710 (2003); Haas et al.; Viruses 5, 87-110 (2013)). Initially identified in 2001 in samples collected from children with respiratory tract infection in the Netherlands (van den Hoogen et al Nat. Med. 7, 719-724 (2001)), the clinical features of hMPV are similar to those of respiratory syncytial virus (RSV), and include mid-to-upper respiratory tract infection that may require hospitalization (Akhras et al., Infect. Dis. Rep. 2, e12 (2010)). Severe disease can occur in immunocompromised patients, such as those undergoing lung transplant (Larcher et al., J. Hear. Lung Transpl. 24, 1891-1901 (2005)), hematopoietic stem cell transplant (Cane et al., Bone Marrow Transplant. 31, 309-310 (2003); Englund et al., Ann. Intern. Med. 144, 344-349 (2013); Dokos et al., Transpl. Infect. Dis. 15, 97-101 (2013); Shah, et al., Cancer Lett. 379, 100-106 (2016)), as well as those living with HIV (Klein et al., J. Infect. Dis. 201, 297-301 (2010)) and COPD (Kan-o et al., J. Infect. Dis. 215, 1536-1545 (2018)). There are no approved vaccines or specific treatments available for hMPV infection, in contrast to RSV, for which palivizumab (Group and Im, Pediatrics 102, 531-537 (1998)) has been in use for many years in specific high-risk infant groups.

Serological studies have shown that nearly all children are seropositive for hMPV by five years of age (Edwards et al. N. Engl. J. Med. 368, 633-643 (2013)). hMPV has three surface glycoproteins—the small hydrophobic (SH), attachment (G), and fusion (F) proteins. Of these, the hMPV F protein is the only target of neutralizing antibodies (Ulbrandt et al., J. Gen. Virol. 89, 3113-3118 (2008)), which is different than RSV where both the RSV G and RSV F proteins elicit neutralizing antibodies (Tripp et al., J. Virol. 92, 1-8 (2017)). There are no licensed vaccines to prolect against bMPV, but several candidates have been examined in animal models, including live-attenuated viruses, recombinam viruses, vectored vaccines, and recombinant surface proteins (Shafagati and Williams, F1000Res. 7, 135 (2018)). Limited vaccine candidates have advanced to clinical trials, including a live-attenuated hMPV vaccine (NCT1255410), and more recently, an mRNA-based vaccine combined with parainfluenza virus 3 (NCT03392389, NCTO4144348). Similar to vaccine enhanced disease observed with formalin-inactivated RSV (Killikelly et al. Sci. Rep. 6, 34108 (2016); Kim et al. Am. J. Epidemiol. 89, 422-434 (1969); Kapikian et al., Am. J. Epidemiol. 89, 405-421 (1969)), vaccines using formalin-inactivated hMPV result in enhanced disease following viral infection in mice, cotton rats, and macaques (Hamelin et al., J. Gen. Virol. 88, 3391-3400 (2007); Yim et al. Vaccine 25, 5034-5040 (2007)). Thus, a need remains for other therapeutics targeting hMPV.

### SUMMARY OF THE DISCLOSURE

Disclosed is an isolated monoclonal antibody or antigen binding fragment thereof, that includes a heavy chain variable (V_{H}) region and a light chain variable region (V_{L}) comprising a heavy chain complementarity determining region (HCDR)1, a HCDR2, and a HCDR3, and a light chain complementarity determining region (LCDR)1, a LCDR2, and a LCDR3 of the V_{H} and V_{L} set forth as:
a) SEQ ID NOs: 41 and 55, respectively;
b) SEQ ID NOs: 9 and 13, respectively;
c) SEQ ID NOs: 17 and 21, respectively;
d) SEQ ID NOs: 25 and 29, respectively;
e) SEQ ID NOs: 33 and 37, respectively;
f) SEQ ID NOs: 1 and 5, respectively;
g) SEQ ID NOs: 49 and 53, respectively;
h) SEQ ID NOs: 57 and 61, respectively;
i) SEQ ID NOs: 65 and 69, respectively;
j) SEQ ID NOs: 73 and 77, respectively;
k) SEQ ID NOs: 81 and 85, respectively;
l) SEQ ID NOs: 89 and 93, respectively;
m) SEQ ID NOs: 97 and 101, respectively;
n) SEQ ID NOs: 105 and 109, respectively;
o) SEQ ID NOs: 113 and 117, respectively;
p) SEQ ID NOs: 121 and 125, respectively;
q) SEQ ID NOs: 129 and 133, respectively; or
r) SEQ ID NOs: 137 and 141, respectively;
wherein the monoclonal antibody specifically binds to human metapneumovirus (hMPV) F protein and neutralizes hMPV. In specific non-limiting examples, the CDRs can be identified using the e Kabat, Chothia or IMGT numbering scheme.

In some embodiments, disclosed is an isolated monoclonal antibody or antigen binding fragment thereof, wherein the HCDR1, the HCDR2, the HCDR3, the LCDR1, the LCDR2, and the LCDR3 comprise the amino acids sequences set forth as:
a) SEQ ID NOs: 42, 43, 44, 46, 47, and 48, respectively;
b) SEQ ID NOs: 10, 11, 12, 14, 15, and 16, respectively;
c) SEQ ID NOs: 18, 19, 20, 22, 23, and 24, respectively;
d) SEQ ID NOs: 26, 27, 28, 30, 31, and 32, respectively;
e) SEQ ID NOs: 34, 35, 36, 38, 39, and 40, respectively;
f) SEQ ID NOs: 2, 3, 4, 6, 7, and 8, respectively;
g) SEQ ID NOs: 50, 51, 52, 54, 55, and 56, respectively;
h) SEQ ID NOs: 58, 59, 60, 62, 63, and 64, respectively;
i) SEQ ID NOs: 66, 67, 68, 70, 71, and 72, respectively;
j) SEQ ID NOs: 74, 75, 76, 78, 79, and 80, respectively;
k) SEQ ID NOs: 82, 83, 84, 86, 87, and 88, respectively;
l) SEQ ID NOs: 90, 91, 92, 94, 95, and 96, respectively;
m) SEQ ID NOs: 98, 99, 100, 102, 103, and 104, respectively;
n) SEQ ID NOs: 106, 107, 108, 110, 111, and 112, respectively;
o) SEQ ID NOs: 114, 115, 116, 118, 119, and 120, respectively;
p) SEQ ID NOs: 122, 123, 124, 126, 127, and 128, respectively;
q) SEQ ID NOs: 130, 131, 132, 134, 135, and 136, respectively; or
r) SEQ ID NOs: 138, 139, 140, 142, 143, and 144, respectively.

In some embodiments, the antibody or antigen binding fragment is conjugated to an effector molecule or a detectable marker.

Bispecific antibodies comprising the antibody or antigen binding fragment are also disclosed.

In further embodiments, nucleic acids and vectors encoding the antibody, antigen binding fragment, or a V_{H} or V_{L} of the antibody or antigen binding fragment.

In more embodiments, pharmaceutical compositions are disclosed for use in inhibiting an hMPV infection that includes an effective amount of the antibody, antigen binding fragment, nucleic acid molecule, or vector, and a pharmaceutically acceptable carrier.

In further embodiments, disclosed is a method of producing an antibody or antigen binding fragment that specifically binds to hMPV F protein.

In some embodiments, methods are also disclosed for detecting the presence of hMPV in a biological sample from a human subject.

In additional embodiments, methods are disclosed for inhibiting an hMPV infection in a subject, comprising administering an effective amount of the antibody, antigen binding fragment, nucleic acid molecule, vector, or pharmaceutical composition to the subject, wherein the subject has or is at risk of an hMPV infection.

The foregoing and other features and advantages of the invention will become more apparent from the following detailed description of several embodiments which proceeds with reference to the accompanying figures.

### BRIEF DESCRIPTION OF THE FIGURES

FIGS. 1A-1C. Sequence determinants of the isolated mAbs. (A) The usage of heavy, kappa, and lambda chain genes are shown as a proportion of all respective genes from the panel of isolated mAbs. (B) The amino acid lengths of the junction for the heavy and light chains are shown. (C) The percent identity of the V gene to predicted germline sequences are shown.

FIGS. 2A-2C. Epitope mapping of the hMPV F-specific mAbs. (A) Epitope binning for mAbs binding to the hMPV B2 F protein. Data indicate the percent binding of the second antibody in the presence of the primary antibody, as compared to the second antibody alone. Cells are colored in a gradient according to the legend displayed right. (B) Epitopes for control mAbs 101F (site IV), 196 and DS7 (DS7 epitope), MPE8 and MPV364 (site III), and MPV458 (66-87 epitope) were used as the second mAb and are labeled according to the colors in (A). Select control mAbs were also used as the first mAb as positive blocking controls. (C) The mAb binding sites of MPE8 and MPV364 (site III), 101F (site IV), DS7, and MPV458 (66-87 epitope) are displayed on the surface of monomeric prefusion hMPV F. Estimated epitopes from epitope binning for each mAb are displayed. mAbs with an asterisk were previously discovered.

FIGS. 3A-3B. The percent phagocytosis of hMPV F-coated beads by THP-1 cells in the presence of each mAb was assessed using flow cytometry. The relative percent increase of phagocytic cells for each mAb relative to the no mAb control (A), in addition to the phagocytic score (B), are shown. Bars represent the average of three replicates, while errors bars are the standard deviation.

FIGS. 4A-4C. Protective efficacy of MPV467 against hMPV replication in vivo. (A) BALB/c mice were treated intraperitoneally with 10 mg/kg of mAb MPV467 24 his prior (prophylaxis study) or three days after (treatment study) intranasal hMPV infection. Viral titers in the lung homogenates of BALB/c mice in each treatment group (n=10 mice per group, 5 males, 5 females) in prophylaxis study (B) and treatment study (C) were determined by plaque assay. n.s.=not significant, **P=0.0016, ***P=0.0003-0.0001, ****P<0.0001. Limit of detection (LOD) is indicated with a dashed line. ND=not detected.

FIGS. 5A-5C. MPV467 binds pre-fusion hMPV F at sites II and V. (A) (left) Side view of the hMPV F-MPV467 Fab complex cryo-EM map shown at two different contour levels. Global map shown as white transparent map. Particle-subtracted, DeepEMhanced map is opaque with a single protomer identified (right) Top-down view of particle-subtracted, DeepEMhanced map. (B) A single protomer of the hMPV F trimer and MPV467 Fab variable domain are shown as ribbons (hMPV F: blue, Fab: red/orange). (C) Zoomed in view of the MPV467 interface with hMPV F. View direction as shown by box and arrow in panel (B). Important residues shown as sticks. Hydrogen bonds and salt bridges depicted as black dotted lines. Oxygen atoms are colored red and nitrogens are blue.

FIG. 6. Neutralization profiles of the hMPV F protein-specific mAbs. Data represent the averages from three replicates, and error bars are the standard deviation. Data are representative of results from at least two independent experiments.

FIG. 7. ELISA binding curves of the hMPV F protein-specific mAbs. ELISA binding curves of the isolated mAbs with recombinant hMPV F protein constructs. Prefusion RSV F protein (DsCav1) was utilized to determine if any mAb cross-reacts with RSV F. The previously discovered hMPV F-specific mAb MPV196 was used as a negative binding control. Each point represents the average of data from four replicates, and error bars represent the standard deviation. Data are representative of results from at least two independent experiments.

FIG. 8. MPV467 cryo-EM processing workflow. Each step, from representative micrograph to DeepEMhancer map, of the cryo-EM data processing workflow is shown. Computational programs and algorithms used are labeled for each step. The mask used for particle subtraction is colored as a transparent cyan.

FIGS. 9A-9E. MPV467 cryo-EM Validation. (A) (top) FSC curves for the homogeneous refinement 3D reconstruction. Horizontal blue line corresponds to an FSC value of 0.143. (bottom) Viewing distribution plot calculated in cryoSPARC. (B) FSC curves and viewing distribution plot for the particle-subtracted non-uniform refinement 3D reconstruction. (top) The FSC curves for the non-uniform refinement 3D reconstruction. The horizontal blue line corresponds to an FSC value of 0.143. (bottom) Viewing distribution plot calculated in cryoSPARC. (C) Cryo-EM maps of homogenous refinement (left) and particle-subtracted non-uniform refinement (right) colored by local resolution. Cryo-EM maps are shown as top (top) and side (bottom) views. (D) The cryo-EM map of DS-CavEs2-IPDS bound by MPV467 is shown in a side view (left) and top view (right). A single protomer is shown as a transparent colored surface with a docked ribbon model (DS-CavEs2-IPDS: blue; MPV467 heavy chain: red, MPV467 light chain: orange). (E) The binding interface for MPV467 heavy chain (left) and light chain (right) with DS-CavEs2-IPDS. Cryo-EM map is shown as a transparent surface with the docked model shown as ribbon and sticks. Colored the same as in D.

FIG. 10 is Table 1. hMPV F-specific mAb neutralization and binding properties. Neutralization values were determined using a plaque-reduction assay, where the IC₅₀ corresponds to the mAb concentration at which 50% plaque reduction was observed. EC₅₀ values correspond to the concentration at which the half-maximum signal was obtained in ELISA, based on the optical density at 405 nm. >indicates the binding signal was below 1 μg/mL at the highest mAb concentration tested. Each value is an average of three technical replicates for neutralization experiments and four technical replicates for binding experiments. Each experiment was repeated independently at least twice.

FIG. 11 is Table 2. EM data collection for MPV467.

FIG. 12. hMPV replication and clearance in immunosuppressed vs. normal cotton rats. Cotton rats immunosuppressed via repeated cyclophosphamide treatments (Immunosuppr) or normal cotton rats (Normal) were challenged with hMPV at 10⁵ PFU per animal. Five, seven, or nine days after infection animals were sacrificed, and lung and nose samples were collected for viral titration by plaque assay followed by immunostaining. Results represent the geomean±S.E.M. for 4-10 animals per group (data combined from two studies). *p<0.05 when compared to hMPV-infected normal animals sacrificed on the same day.

FIG. 13. MPV467 prophylaxis and therapy of hMPV infection in immunosuppressed cotton rats: dose-dependency of antiviral effect. Immunosuppressed S. hispidus were challenged with hMPV at 10⁵ PFU/animal. MPV467 treatment was administered intramuscularly as 0.1, 1, or 10 mg/kg one day before (Prophyl) or three days after (Therap) hMPV challenge. Five days after infection, animals were sacrificed and lung and nose samples were collected for hMPV quantification. Results represent the geomean±S.E.M. for 5 animals per group. *p<0.05 when compared to hMPV-infected mock-treated animals sacrificed on day five post-infection.

FIG. 14. Effect of antibody therapy on delayed viral clearance in immunosuppressed cotton rats infected with hMPV. Immunosuppressed and normal S. hispidus were challenged with hMPV at 10⁵ PFU/animal and treated with MPV467 10 mg/kg on day three (normal) and days three and seven (immunosuppressed) after infection. Five or nine days after infection animals were sacrificed and lung and nose samples were collected for hMPV quantification. Results represent the geomean±S.E.M. for 4-10 animals per group. *p<0.05 when compared to hMPV-infected mock-treated animals sacrificed on the corresponding day.

FIG. 15. Effect of antibody therapy on lung histopathology in normal and immunosuppressed cotton rats infected with hMPV. Immunosuppressed and normal S. hispidus were challenged with hMPV at 10⁵ PFU/animal and treated with MPV467 as described in the description of FIG. 14. Pulmonary histopathology was evaluated in hematoxylin-eosin (H&E) slides in each of the following categories: peribronchiolitis (Peribr), perivasculitis (Perivasc), interstitial inflammation (Interst), alveolitis (Alveol), and epithelial hyperplasia (Epith.Hyp.). Results represent the mean±SE for 4-5 animals per group.

FIG. 16. Effect of antibody therapy on pulmonary cytokine/chemokine expression in normal and immunosuppressed cotton rats infected with hMPV. Immunosuppressed and normal S. hispidus were challenged with hMPV at 10⁵ PFU/animal, treated with 10 mg/kg MPV467 or PBS (mock), and sacrificed 9 days post-infection. Expression of MIP-la and IP-10 mRNA was quantified in the lung tissue by qPCR and normalized by the expression of b-actin in the corresponding organ. Results represent the mean±SE for 4-5 animals per group. *p<0.05 compared to normal animals uninfected (Ctl) or receiving the PBS (mock) or MPV467 (Ab) treatment. #p<0.05 for antibody-vs. mock-treated animals.

### DETAILED DESCRIPTION OF SEVERAL EMBODIMENTS

The hMPV F protein is a trimreric class I viral fusion protein that has high conservation between viral subgroups (A1, A2, B1, B2) (Huang and Mousa, Front. Immunol. 10, 2778 (2019)). hMPV can infect respiratory epithelial cells in the absence of the hMPV G protein, although hMPV G is required for viral fitness in vivo (Biacchesi et al., J. Virol. 78, 12877-12887 (2004)). The bMPV F protein contains an RGD motif, and the receptor has been hypothesized to be α₅β₁ integrin (Cox et al., J. Virol. 86, 12148-12160 (2012)). Heparan sulfate has also been shown to have a role in hMPV F protein-mediated attachment (Chang et al., J. Virol. 86, 3230-3243 (2012)), and direct binding was recently demonstrated between heparan sulfate and the hMPV F protein (Jiachen, H. et al. J. Virol. 95, e00593-21 (2021)). hMPV F induces fusion of viral and host cell membranes in a transition from the metastable pre-fusion state to the post-fusion conformation (Poor et al., PNAS 111, 2596-2605 (2014)). X-ray crystal structures of the hMPV F protein in the pre-fusion (Battles et al. Nat. Commun. 8, 1528 (2017)) and post-fusion (Jiachen, H. et al. J. Virol. 95, e00593-21 (2021); Mds et al. PLoS Pathog. 12, e1005859 (2016)) conformations have been elucidated, and the protein shares similar structural topology with the RSV F protein (McLellan et al., Curr. Top. Microbiol. Immunol. 372, 83-104 (2013)).

There has been a paucity of information regarding specific epitopes on the hMPV F protein compared to RSV F. Clear differences in the immunologic features between RSV F and hMPV F have been identified. For example, pre-fusion and post-fusion hMPV F proteins elicit similar antibody responses, suggesting that the majority of the neutralizing epitopes are present in both conformations (Jiachen, H. et al. J. Virol. 95, e00593-21 (2021); Battles et al. Nat. Commun. 8, 1528 (2017); Pilaev et al., Vaccine 38, 2122-2127 (2020)), while for RSV F the majority and the most potent neutralizing antibodies target pre-fusion-specific epitopes (Huang and Mousa, Front. Immunol. 10, 2778 (2019); Gilman et al., Sci. Immunol. 1, 1-12 (2016); Mousa et al., Nat. Microbiol. 2, 16271 (2017). Known neutralizing epitopes on the hMPV F protein include antigenic sites IV (Mas et al. PLoS Pathog. 12, e1005859 (2016); Mousa et al., PLoS Pathog. 14, e1006837 (2018); Schuster et al., J. Infect. Dis. 211, 1-34 (2014)), 111 (Corti et al., Nature 501, 439-43 (2013); Wen et al., Nat. Microbiol. 2, 16272 (2017)) and V (Xiao et al., MAbs 11, 1415-1427 (2019)) based upon identification of RSV F mAbs that also neutralize hMPV F. A unique epitope targeted by the human mAb DS7 has been structurally defined (Wen et al. Nat. Struc. Mol. Biol. 19, 461-463 (2012)). Additionally, a novel epitope located within the trimeric interface of the bMPV F protein, which was defined by mAb MPV458, was recently identified (Huang and Mousa, PLOS Pathog. 16, e1008942 (2020)). To further the understanding of neutralizing hMPV F epitopes, a panel of 18 human mAbs to the hMPV F protein were isolated.

Two mAbs MPV467 and MPV487, were demonstrated to have potent neutralizing activity, with MPV467 being exceptionally potent. It was also demonstrated that MPV467 can prevent and treat bMPV infection in mice, and using cryo-electron microscopy it was determined that the epitope of MPV467 targets a complex binding site interfacing both antigenic sites I and V.

### I. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

About: Unless context indicated otherwise, “about” refers to plus or minus 5% of a reference value. For example, “about” 100 refers to 95 to 105.

Administration: The introduction of a composition into a subject by a chosen route. Administration can be local or systemic. For example, if the chosen route is intravenous, the composition (such as a composition including a disclosed antibody or antigen binding fragment) is administered by introducing the composition into a vein of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Agent: Any substance or any combination of substances that is useful for achieving an end or result; for example, a substance or combination of substances useful for inhibiting an hMPV infection in a subject. Agents include proteins, nucleic acid molecules, compounds, small molecules, organic compounds, inorganic compounds, or other molecules of interest. An agent can include a therapeutic agent (such as an anti-retroviral agent), a diagnostic agent or a pharmaceutical agent. In some embodiments, the agent is an antibody that specifically bind hMPV, optionally combined with an anti-viral agent. The skilled artisan will understand that particular agents may be useful to achieve more than one result.

Amino acid substitutions: The replacement of one amino acid in a polypeptide with a different amino acid or with no amino acid (i.e., a deletion). In some examples, an amino acid in a polypeptide is substituted with an amino acid from a homologous polypeptide, for example, and amino acid in a recombinant group A MPV F polypeptide can be substituted with the corresponding amino acid from a group B MPV F polypeptide.

Antibody and Antigen Binding Fragment: An immunoglobulin, antigen-binding fragment, or derivative thereof, that specifically binds and recognizes an analyte (antigen) such as an hMPV F polypeptide. The term “antibody” is used herein in the broadest sense and encompasses various antibody structures, including but not limited to monoclonal antibodies, polyclonal antibodies, multispecific antibodies (e.g., bispecific antibodies), and antigen binding fragments, so long as they exhibit the desired antigen-binding activity.

Non-limiting examples of antibodies include, for example, intact immunoglobulins and variants and fragments thereof that retain binding affinity for the antigen. Examples of antigen binding fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies (see, e.g., Kontermann and Dubel (Eds.), Antibody Engineering, Vols. 1-2, 2^{nd} ed., Springer-Verlag, 2010).

Antibodies also include genetically engineered forms such as chimeric antibodies (such as humanized murine antibodies) and heteroconjugate antibodies (such as bispecific antibodies).

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

Typically, a naturally occurring immunoglobulin has heavy (H) chains and light (L) chains interconnected by disulfide bonds. Immunoglobulin genes include the kappa, lamda, alpha, gamma, delta, epsilon and mu constant region genes, as well as the myriad immunoglobulin variable domain genes. There are two types of light chain, lamda (λ) and kappa (κ). There are five main heavy chain classes (or isotypes) which determine the functional activity of an antibody molecule: IgM, IgD, IgG, IgA and IgE.

Each heavy and light chain contains a constant region (or constant domain) and a variable region (or variable domain). In combination, the heavy and the light chain variable regions specifically bind the antigen.

References to “V_{H}” or “V_{H}” refer to the variable region of an antibody heavy chain, including that of an antigen binding fragment, such as Fv, scFv, dsFv or Fab. References to “V_{L}” or “VL” refer to the variable domain of an antibody light chain, including that of an Fv, scFv, dsFv or Fab.

The V_{H} and V_{L} contain a “framework” region interrupted by three hypervariable regions, also called “complementarity-determining regions” or “CDRs” (see, e.g., Kabat et al., Sequences of Proteins of Immunological Interest, 5^{th} ed., NIH Publication No. 91-3242, Public Health Service, National Institutes of Health. U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (Sequences of Proteins of Immunological Interest, 5^{th} ed., NIH Publication No. 91-3242, Public Health Service, National Institutes of Health, U.S. Department of Health and Human Services, 1991; “Kabat” numbering scheme), Al-Lazikani et al., (“Standard conformations for the canonical structures of immunoglobulins,” J. Mol. Bio., 273(4):927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27(1):55-77, 2003; “IMGT” numbering scheme). The CDRs of each chain are typically referred to as CDR1, CDR2, and CDR3 (from the N-terminus to C-terminus), and are also typically identified by the chain in which the particular CDR is located. Thus, a V_{H} CDR3 is the CDR3 from the V_{H} of the antibody in which it is found, whereas a V_{L} CDR1 is the CDR1 from the V_{L} of the antibody in which it is found. Light chain CDRs are sometimes referred to as LCDR1, LCDR2, and LCDR3. Heavy chain CDRs are sometimes referred to as HCDR1, HCDR2, and HCDR3.

In some embodiments, a disclosed antibody includes a heterologous constant domain. For example, the antibody includes a constant domain that is different from a native constant domain, such as a constant domain including one or more modifications (such as the “LS” mutations) to increase half-life.

A “monoclonal antibody” is an antibody obtained from a population of substantially homogeneous antibodies, that is, the individual antibodies comprising the population are identical and/or bind the same epitope, except for possible variant antibodies, for example, containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In some examples monoclonal antibodies are isolated from a subject. Monoclonal antibodies can have conservative amino acid substitutions which have substantially no effect on antigen binding or other immunoglobulin functions. (See, for example, Greenfield (Ed.), Antibodies: A Laboratory Manual, 2^{nd} ed. New York: Cold Spring Harbor Laboratory Press, 2014.)

A “humanized” antibody or antigen binding fragment includes a human framework region and one or more CDRs from a non-human (such as a mouse, rat, or synthetic) antibody or antigen binding fragment. The non-human antibody or antigen binding fragment providing the CDRs is termed a “donor,” and the human antibody or antigen binding fragment providing the framework is termed an “acceptor.” In one embodiment, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

A “chimeric antibody” is an antibody which includes sequences derived from two different antibodies, which typically are of different species. In some examples, a chimeric antibody includes one or more CDRs and/or framework regions from one human antibody and CDRs and/or framework regions from another human antibody.

A “fully human antibody” or “human antibody” is an antibody which includes sequences from (or derived from) the human genome, and does not include sequence from another species. In some embodiments, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1^{st} Ed. New York: Cold Spring Harbor Laboratory Press, 2004. Print.; Lonberg, Nat. Biotech., 23: 1117-1125, 2005; Lonenberg, Curr. Opin. Immunol., 20:450-459, 2008).

Antibody or antigen binding fragment that neutralizes MPV: An antibody or antigen binding fragment that specifically binds to an hMPV antigen, such as hMPV (for example, an F protein) in such a way as to inhibit a biological function associated with that inhibits the hMPV infection. The antibody can neutralize the activity of hMPV at various points during the lifecycle of the pathogen.

Biological sample: A sample obtained from a subject. Biological samples include all clinical samples useful for detection of disease or infection (for example, an hMPV infection) in subjects, including, but not limited to, cells, tissues, and bodily fluids, such as blood, derivatives and fractions of blood (such as serum), cerebrospinal fluid; as well as biopsied or surgically removed tissue, for example tissues that are unfixed, frozen, or fixed in formalin or paraffin. In a particular example, a biological sample is obtained from a subject having or suspected of having a hMPV infection.

Bispecific antibody: A recombinant molecule composed of two different antigen binding domains that consequently binds to two different antigenic epitopes. Bispecific antibodies include chemically or genetically linked molecules of two antigen-binding domains. The antigen binding domains can be linked using a linker. The antigen binding domains can be monoclonal antibodies, antigen-binding fragments (e.g., Fab, scFv), or combinations thereof. A bispecific antibody can include one or more constant domains but does not necessarily include a constant domain.

Conditions sufficient to form an immune complex: Conditions which allow an antibody or antigen binding fragment thereof to bind to its cognate epitope to a detectably greater degree than, and/or to the substantial exclusion of, binding to substantially all other epitopes. Conditions sufficient to form an immune complex are dependent upon the format of the binding reaction and typically are those utilized in immunoassay protocols or those conditions encountered in vivo. See Harlow & Lane, Antibodies, A Laboratory Manual, 2^{nd} ed. Cold Spring Harbor Publications, New York (2013) for a description of immunoassay formats and conditions. The conditions employed in the methods are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (e.g., from pH 6.0 to pH 8.0, more typically pH 6.5 to 7.5), contains water as the predominant solvent, and exists at a temperature above 0° C., and below 50° C. Osmolarity is within the range that is supportive of cell viability and proliferation.

Conjugate: A complex of two molecules linked together, for example, linked together by a covalent bond. In one embodiment, an antibody is linked to an effector molecule; for example, an antibody that specifically binds to an hMPV F protein covalently linked to an effector molecule. The linkage can be by chemical or recombinant means. In one embodiment, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. Because conjugates can be prepared from two molecules with separate functionalities, such as an antibody and an effector molecule, they are also sometimes referred to as “chimeric molecules.”

Conservative variants: “Conservative” amino acid substitutions are those substitutions that do not substantially affect or decrease a function of a protein, such as the ability of the protein to interact with a target protein. For example, a MPV-specific antibody can include up to 1, 2, 3, 4, 5, 6, 7, 8, 9, or up to 10 conservative substitutions compared to a reference antibody sequence and retain specific binding activity for MPV, and/or MPV neutralization activity. The term conservative variation also includes the use of a substituted amino acid in place of an unsubstituted parent amino acid.

Individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of the hMPV specific antibody, such as the ability to specifically bind to hMPV F protein or neutralize hMPV. For instance, if an amino acid residue is essential for a function of the protein, even an otherwise conservative substitution may disrupt that activity. Thus, a conservative substitution does not alter the basic function of a protein of interest.

Contacting: Placement in direct physical association; includes both in solid and liquid form, which can take place either in vivo or in vitro. Contacting includes contact between one molecule and another molecule, for example the amino acid on the surface of one polypeptide, such as a peptide, that contacts another polypeptide. Contacting can also include contacting a cell for example by placing a polypeptide in direct physical association with a cell.

Control: A reference standard. In some embodiments, the control is a negative control sample obtained from a healthy patient. In other embodiments, the control is a positive control sample obtained from a patient diagnosed with MPV infection. In still other embodiments, the control is a historical control or standard reference value or range of values (such as a previously tested control sample, such as a group of MPV patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Degenerate variant: In the context of the present disclosure, a “degenerate variant” refers to a polynucleotide encoding a polypeptide (such as a recombinant MPV F protein or immunogenic fragment thereof) that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Detectable marker: A detectable molecule (also known as a label) that is conjugated directly or indirectly to a second molecule, such as an antibody, to facilitate detection of the second molecule. For example, the detectable marker can be capable of detection by ELISA, spectrophotometry, flow cytometry, microscopy or diagnostic imaging techniques (such as CT scans, MRIs, ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). Methods for using detectable markers and guidance in the choice of detectable markers appropriate for various purposes are discussed for example in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements, 2017).

Detecting: To identify the existence, presence, or fact of something.

DS7 Antibody: A neutralizing monoclonal antibody that specifically binds to an epitope on hMPV F protein that is present on the pre- and post-fusion conformations of the hMPV F protein. The DS7 antibody does not specifically bind to hMPV F in its postfusion conformation. The DS7 antibody and methods for its production are described, for example, in Wen et al., Nat. Struct. Mol. Biol., 19, 461-463, 2012, which is incorporated by reference herein in its entirety. The amino acid sequences of the heavy and light variable regions of the DS7 antibody are provided as SEQ ID NOs: 41 and 42, and have been deposited in PDB as Nos. 4DAG_H (DS7 V_{H}) and 4DAG_L (DS7 V_{L}), each of which is incorporated by reference herein as resent in the database on Nov. 10, 2014).

DS7 V_{H}-
(SEQ ID NO: 145)
EVQLLESGGGLVQPGGSRRLSCAASGFTVSSSYMSWVRQTPGKGLEWIS
VFYSGGTTYYADAVKGRFSISMDTSKNTLHLQMNSLRVEDTAIYYCARV
LSRASGMPDAFDIWGPGTMVTVSS
DS7 V_{L}-
(SEQ ID NO: 146)
ELALIQPASVSVSPGQTASITCSGDKLGDKYASWYQQKPGQSPVLVIYQ
DSERPSGIPERFSGSNSGNTATLTISGTQAMDEADYYCQAWDSSTAVFG
GGTTLTVLGQ

Effective amount: A quantity of a specific substance sufficient to achieve a desired effect in a subject to whom the substance is administered. For instance, this can be the amount of an antibody necessary to inhibit an hMPV infection, or to measurably alter outward symptoms of the hMPV infection.

In some embodiments, administration of an effective amount of a disclosed antibody or antigen binding fragment that binds to an hMPV F protein can reduce or inhibit an MPV infection (for example, as measured by infection of cells, or by number or percentage of subjects infected by the hMPV, or by an increase in the survival time of infected subjects, or reduction in symptoms associated with the hMPV) by a desired amount, for example by at least 10%, at least 20%, at least 50%, at least 60%, at least 70%, at least 80%, at least 90%, at least 95%, at least 98%, or even at least 100% (elimination or prevention of detectable hMPV infection), as compared to a suitable control.

The effective amount of an antibody or antigen binding fragment that specifically binds to the hMPV F protein that is administered to a subject to inhibit an hMPV infection will vary depending upon a number of factors associated with that subject, for example the overall health and/or weight of the subject. An effective amount can be determined by varying the dosage and measuring the resulting response, such as, for example, a reduction in pathogen titer. Effective amounts also can be determined through various in vitro, in vivo or in situ immunoassays.

An effective amount encompasses a fractional dose that contributes in combination with previous or subsequent administrations to attaining an effective response. For example, an effective amount of an agent can be administered in a single dose, or in several doses, for example daily, during a course of treatment lasting several days or weeks. However, the effective amount can depend on the subject being treated, the severity and type of the condition being treated, and the manner of administration. A unit dosage form of the agent can be packaged in an amount, or in multiples of the effective amount, for example, in a vial (e.g., with a pierceable lid) or syringe having sterile components.

Epitope: An antigenic determinant. These are particular chemical groups or peptide sequences on a molecule that are antigenic, such that they elicit a specific immune response, for example, an epitope is the region of an antigen to which B and/or T cells respond. An antibody can bind to a particular antigenic epitope, such as an epitope on hMPV F protein.

Expression: Transcription or translation of a nucleic acid sequence. For example, a gene is expressed when its DNA is transcribed into an RNA or RNA fragment, which in some examples is processed to become mRNA. A gene may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. The term “expression” is used herein to denote either transcription or translation. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lamda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

Fc region: The constant region of an antibody excluding the first heavy chain constant domain. Fe region generally refers to the last two heavy chain constant domains of IgA, IgD, and IgG, and the last three heavy chain constant domains of IgE and IgM. An Fc region may also include part or all of the flexible hinge N-terminal to these domains. For IgA and IgM, an Fc region may or may not include the tailpiece, and may or may not be bound by the J chain. For IgG, the Fc region is typically understood to include immunoglobulin domains Cγ2 and Cγ3 and optionally the lower part of the hinge between Cγ1 and Cγ2. Although the boundaries of the Fc region may vary, the human IgG heavy chain Fc region is usually defined to include residues following C226 or P230 to the Fc carboxyl-terminus, wherein the numbering is according to Kabat. For IgA, the Fc region includes immunoglobulin domains Cα2 and Cα3 and optionally the lower part of the hinge between Cα1 and Cα2.

Heterologous: Originating from a different genetic source. A nucleic acid molecule that is heterologous to a cell originated from a genetic source other than the cell in which it is expressed. In one specific, non-limiting example, a heterologous nucleic acid molecule encoding a protein, such as an scFv, is expressed in a cell, such as a mammalian cell. Methods for introducing a heterologous nucleic acid molecule in a cell or organism are well known in the art, for example transformation with a nucleic acid, including electroporation, lipofection, particle gun acceleration, and homologous recombination.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

IgG: A polypeptide belonging to the class or isotype of antibodies that are substantially encoded by a recognized immunoglobulin gamma gene. In humans, this class comprises IgG₁, IgG₂, IgG₃, and IgG₄.

Immune complex: The binding of antibody or antigen binding fragment (such as a scFv) to a soluble antigen forms an immune complex. The formation of an immune complex can be detected through conventional methods, for instance immunohistochemistry, immunoprecipitation, flow cytometry, immunofluorescence microscopy, ELISA, immunoblotting (for example, Western blot), magnetic resonance imaging, CT scans, radiography, and affinity chromatography.

Immune response: A response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies.

Immunogen: A compound, composition, or substance that can stimulate the production of antibodies or a T cell response in an animal, including compositions that are injected or absorbed into an animal, such as hMPV. An immunogen reacts with the products of specific humoral or cellular immunity.

Inhibiting a disease or condition: Reducing the full development of a disease or condition in a subject, for example, reducing the full development of an MPV infection, such as a hMVP infection, in a subject who is at risk of an MPV infection. This includes neutralizing, antagonizing, prohibiting, restraining, slowing, disrupting, stopping, or reversing progression or severity of the disease or condition.

Inhibiting a disease or condition can refer to a prophylactic intervention administered before the disease or condition has begun to develop (for example a treatment initiated in a subject at risk of an hMPV infection, but not infected by hMPV) that reduces subsequent development of the disease or condition and/or ameliorates a sign or symptom of the disease or condition following development. The term “ameliorating,” with reference to inhibiting a disease or condition refers to any observable beneficial effect of the prophylactic intervention intended to inhibit the disease or condition. The beneficial effect can be evidenced, for example, by a delayed onset of clinical symptoms of the disease or condition in a susceptible subject, a reduction in severity of some or all clinical symptoms of the disease or condition, a slower progression of the disease or condition, an improvement in the overall health or well-being of the subject, a reduction in infection, or by other parameters that are specific to the particular disease or condition.

In some embodiments, the disclosed hMPV F protein-specific antibodies and antigen binding fragments inhibit the growth of the hMPV in a subject, for example, the antibodies and antigen binding fragments inhibit the multiplication of hMPV in the subject, resulting in a reduction in pathogen load in the subject compared to a relevant control. For example, the disclosed hMPV F protein-specific antibodies and antigen binding fragments can inhibit the hMPV infection in a subject, or inhibit viral replication, by at least 20%, at least 30%, at least 40%, or at least 50%, compared to a suitable control.

Isolated: A biological component (such as a nucleic acid, peptide, protein or protein complex, for example an antibody) that has been substantially separated, produced apart from, or purified away from other biological components in the cell of the organism in which the component naturally occurs, that is, other chromosomal and extra-chromosomal DNA and RNA, and proteins. Thus, isolated nucleic acids, peptides and proteins include nucleic acids and proteins purified by standard purification methods. The term also embraces nucleic acids, peptides and proteins prepared by recombinant expression in a host cell, as well as, chemically synthesized nucleic acids. An isolated nucleic acid, peptide or protein, for example an antibody, can be at least 50%, at least 60%, at least 70%, at least 80%, at least 90%, at least 95%, at least 96%, at least 97%, at least 98%, or at least 99% pure.

Kabat position: A position of a residue in an amino acid sequence that follows the numbering convention delineated by Kabat et al. (Sequences of Proteins of Immunological Interest, 5^{th} Edition, Department of Health and Human Services, Public Health Service, National Institutes of Health, Bethesda, NIH Publication No. 91-3242, 1991).

Linker: A bi-functional molecule that can be used to link two molecules into one contiguous molecule, for example, to link an effector molecule to an antibody, or a detectable marker to an antibody. Non-limiting examples of peptide linkers include glycine-serine linkers.

The terms “conjugating,” “joining,” “bonding,” or “linking” can refer to making two molecules into one contiguous molecule; for example, linking two polypeptides into one contiguous polypeptide, or covalently attaching an effector molecule or detectable marker radionuclide or other molecule to a polypeptide, such as an scFv. The linkage can be either by chemical or recombinant means. “Chemical means” refers to a reaction between the antibody moiety and the effector molecule such that there is a covalent bond formed between the two molecules to form one molecule.

Metapneumovirus (MPV): An enveloped non-segmented negative-sense single-stranded RNA virus of the family Paramyxoviridae. It is a common cause of lower respiratory track infections, including bronchiolitis and pneumonia, among children and adults and infects nearly all humans by five years of age. MPV causes repeated infections including severe lower respiratory tract disease, which may occur at any age, especially among the elderly or those with compromised cardiac, pulmonary, or immune systems. A hMPV can infect humans.

The MPV genome includes eight genes encoding nine proteins, including the glycoproteins SH, G and F. The F protein mediates fusion, allowing entry of the virus into the cell cytoplasm. Two groups of human MPV strains have been described, the A and B groups, which are further divided into subgroups A1, A2, B1, and B2. Exemplary MPV strain sequences are known to the person of ordinary skill in the art. Further, several models of human MPV infection are available, including model organisms infected with hMPV (see, e.g., Herfst et al., J General Virol., 88, 2702-2709, 2007; Bayon et al., Rev. Med. Virol., 2, 15-34, 2013; and Liu et al., Clinical Vaccine Immunol., 20, 1246-1254, 2013). The F protein has a head and a tail; the head is the top 50% of the pre-fusion state, and the tail is the bottom 50% of the pre-fusion state.

Methods of diagnosing MPV infection are known, including use of Direct Fluorescent Antibody detection (DFA), Chromatographic rapid antigen detection, and detection of viral RNA using RT PCR. Quantification of viral load can be determined, for example, by Plaque Assay, antigen capture enzyme immunoassay (EIA), or PCR. Quantification of antibody levels can be performed by subgroup-specific neutralization assay or ELISA. Current MPV treatment includes use of the anti-viral Ribaviran and passive administration of experimental monoclonal antibodies such as MPE8 (see, e.g., Corti et al., Nature, 501, 439-443, 2013) and mAb338 (Medimmune, Inc., see Hamelin et al., Antiviral Res., 88, 31-37, 2010), which recognize the MPV F protein and reduces incidence of MPV infection and disease in animal models.

There are several subgroups of MPV, including groups A and B, and subgroups A1, A2, B1, and B2 in human MPV. Within the subgroups of MPV, there are individual strains of each subgroup. Sequences of F proteins from particular MPV strains are known and provided herein (see, e.g., Table 1).

MPV Fusion (F) protein: An MPV envelope glycoprotein that facilitates fusion of viral and cellular membranes. In nature, the MPV F protein is initially synthesized as a single polypeptide precursor approximately 540 amino acids in length, designated F₀. F₀ includes an N-terminal signal peptide that directs localization to the endoplasmic reticulum, where the signal peptide (approximately the first 18 residues of F₀) is proteolytically cleaved. The remaining Foresidues oligomerize to form a trimer which is again processed at a protease site (between approximately F₀ positions 102 and 103; for example, RQSR₁₀₂ (residues 99-102) to generate two disulfide-linked fragments, F₁ and F₂. The smaller of these fragments, F₂, originates from the N-terminal portion of the F₀ precursor and includes approximately residues 20-102 of F₀. The larger of these fragments, F₁, includes the C-terminal portion of the F₀ precursor (approximately residues 103-540) including an extracellular/lumenal region (˜residues 103-490), a transmembrane domain (˜residues 491-513), and a cytoplasmic domain (˜residues 514-540) at the C-terminus.

Three F₂-F₁ protomers oligomerize in the mature F protein, which adopts a metastable “prefusion” conformation that is triggered to undergo a conformational change (to a “postfusion” conformation) upon contact with a target cell membrane. This conformational change exposes a hydrophobic sequence, known as the fusion peptide, which is located at the N-terminus of the F₁ polypeptide, and which associates with the host cell membrane and promotes fusion of the membrane of the virus, or an infected cell, with the target cell membrane.

The extracellular portion of the MPV F protein is the MPV F ectodomain, which includes the F₂ protein (approximately MPV F positions 20-102) and the F₁ ectodomain (approximately MPV F positions 103-490). An MPV F ectodomain trimer includes a protein complex of three MPV F ectodomains.

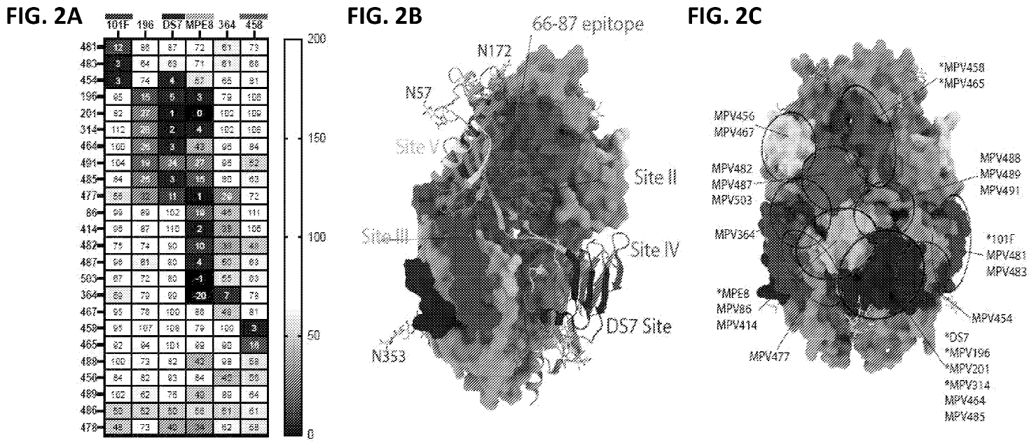

FIGS. 2A-2C show antigenic sites II, III, IV and V of F protein, which are based on competition-binding.

MPV F prefusion conformation: A structural conformation adopted by the MPV F protein prior to triggering of the fusogenic event that leads to transition of MPV F to the postfusion conformation and following processing into a mature MPV F protein in the secretory system. The prefusion conformation of MPV F is similar in overall structure to the prefusion conformation of the F protein of other paramyxoviruses (such as RSV.

MPE8 Antibody: A neutralizing monoclonal antibody that specifically binds to an epitope on MPV F protein that is present on the prefusion, but not the postfusion conformation, of the MPV F protein. The MPE8 antibody and methods for its production are described, for example, in Corti et al. (Nature, 501, 439-443, 2013), which is incorporated by reference herein. MPE8 binds to site III of MPV F protein; site III can be identified by MPE8 binding. The amino acid sequences of the heavy and light variable regions of the MPE8 antibody used herein are provided as SEQ ID NOs: 43 and 44 MPE8 heavy and light chain sequences have been deposited in GenBank as Nos. AGU13651.1 (MPE8 V_{H}) and AGU13652.1 (MPE8 V_{L}), each of which is incorporated by reference herein as present in the database on Nov. 10, 2014).

MPE8 V_{H}-
(SEQ ID NO: 147)
EVQLVESGGGLVKPGGSLRLSCAASGFTFSSYSMNWVRQAPGKGLEWVS
SISASSSYSDYADSAKGRFTISRDNAKTSLFLQMNSLRAEDTAIYFCAR
ARATGYSSITPYFDIWGQGTLVTVSS
MPE8 V_{L}-
(SEQ ID NO: 148)
QSVVTQPPSVSGAPGQRVTISCTGSSSNIGAGYDVHWYQQLPGTAPKLL
IYDNNNRPSGVPDRFSASKSGTSASLAITGLQAEDEADYYCQSYDRSLS
GVFGTGTKVTVL

Neutralizing antibody: An antibody which reduces the infectious titer of an infectious agent by binding to a specific antigen on the infectious agent. In some examples the infectious agent is a virus. In some examples, an antibody that is specific for MPV F neutralizes the infectious titer of hMPV. A “broadly neutralizing antibody” is an antibody that binds to and inhibits the function of related antigens, such as antigens that share at least 85%, 90%, 95%, 96%, 97%, 98% or 99% identity antigenic surface of antigen. With regard to an antigen from a pathogen, such as a virus, the antibody can bind to and inhibit the function of an antigen from more than one class and/or subclass of the pathogen. For example, with regard to MPV, the antibody can bind to and inhibit the function of an antigen, such as MPV F from more than one group.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

A first sequence is an “antisense” with respect to a second sequence if a polynucleotide whose sequence is the first sequence specifically hybridizes with a polynucleotide whose sequence is the second sequence.

Operably linked: A first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the disclosed antibodies and antigen binding fragments thereof.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. In particular embodiments, suitable for administration to a subject the carrier may be sterile, and/or suspended or otherwise contained in a unit dosage form containing one or more measured doses of the composition. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Polypeptide: Any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

Polypeptide modifications: Polypeptides and peptides, such as the antibodies disclosed herein can be modified by a variety of chemical techniques to produce derivatives having essentially the same activity as the unmodified peptides, and optionally having other desirable properties. For example, carboxylic acid groups of the protein, whether carboxyl-terminal or side chain, may be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring. Amino groups of the peptide, whether amino-terminal or side chain, may be in the form of a pharmaceutically-acceptable acid addition salt, such as the HCl, HBr, acetic, benzoic, toluene sulfonic, maleic, tartaric and other organic salts, or may be modified to C₁-C₁₆ alkyl or dialkyl amino or further converted to an amide.

Hydroxyl groups of the peptide side chains can be converted to C₁-C₁₆ alkoxy or to a C₁-C₁₆ ester using well-recognized techniques. Phenyl and phenolic rings of the peptide side chains can be substituted with one or more halogen atoms, such as F, Cl, Br or I, or with C₁-C₁₆ alkyl, C₁-C₁₆ alkoxy, carboxylic acids and esters thereof, or amides of such carboxylic acids. Methylene groups of the peptide side chains can be extended to homologous C₂-C₄ alkylenes. Thiols can be protected with any one of a number of well-recognized protecting groups, such as acetamide groups. Those skilled in the art will also recognize methods for introducing cyclic structures into the peptides of this disclosure to select and provide conformational constraints to the structure that result in enhanced stability. For example, a C- or N-terminal cysteine can be added to the peptide, so that when oxidized the peptide will contain a disulfide bond, generating a cyclic peptide. Other peptide cyclizing methods include the formation of thioethers and carboxyl- and amino-terminal amides and esters.

Purified: The term purified does not require absolute purity; rather, it is intended as a relative term. Thus, for example, a purified peptide preparation is one in which the peptide or protein (such as an antibody) is more enriched than the peptide or protein is in its natural environment within a cell. In one embodiment, a preparation is purified such that the protein or peptide represents at least 50% of the total peptide or protein content of the preparation.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques. A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

Respiratory Syncytial Virus (RSV): An enveloped non-segmented negative-sense single-stranded RNA virus of the family Paramyxoviridae. It is the most common cause of bronchiolitis and pneumonia among children in their first year of life and infects nearly all children by 3 years of age. RSV also causes repeated infections including severe lower respiratory tract disease, which may occur at any age, especially among the elderly or those with compromised cardiac, pulmonary, or immune systems. In the United States, RSV bronchiolitis is the leading cause of hospitalization in infants and a major cause of asthma and wheezing throughout childhood (Shay et al., JAMA, 282, 1440 (1999); Hall et al., N. Engl. J. Med., 360, 588 (2009)). Globally, RSV is responsible for 66,000-199,000 deaths each year for children younger than five years of age (Nair et al., Lancet, 375, 1545 (2010)), and accounts for 6.7% of deaths among infants one month to one year old—more than any other single pathogen except malaria (Lozano et al., Lancet, 380, 2095 (2013)).

The RSV genome is ˜15,000 nucleotides in length and includes 10 genes encoding 11 proteins, including the glycoproteins SH, G and F. The F protein mediates fusion, allowing entry of the virus into the cell cytoplasm and also promoting the formation of syncytia. Two subtypes of human RSV strains have been described, the A and B subtypes, based on differences in the antigenicity of the G glycoprotein. RSV strains for other species are also known, including bovine RSV. Exemplary RSV strain sequences are known to the person of ordinary skill in the art. Further, several models of human RSV infection are available, including model organisms infected with hRSV, as well as model organisms infected with species specific RSV, such as use of bRSV infection in cattle (see, e.g., Bern et al., Am J, Physiol. Lung Cell Mol. Physiol., 301: L148-L156, 2011).

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs, orthologs, or variants of a polypeptide will possess a relatively high degree of sequence identity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natd. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a peptide sequence that has 1166 matches when aligned with a test sequence having 1554 amino acids is 75.0 percent identical to the test sequence (1166±1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least about 75%, for example at least about 80%, 85%, 90%, 91%, 92%, 93%, 94%, 95%, 96%, 97%, 98% or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013). One example of a useful algorithm is PILEUP. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395, 1984.

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (ncbi.nlm.nih.gov). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915, 1989). An oligonucleotide is a linear polynucleotide sequence of up to about 100 nucleotide bases in length.

As used herein, reference to “at least 80% identity” refers to “at least 80%, at least 85%, at least 90%, at least 91%, at least 92%, at least 93%, at least 94%, at least 95%, at least 96%, at least 97%, at least 98%, at least 99%, or even 100% identity” to a specified reference sequence. As used herein, reference to “at least 90% identity” refers to “at least 90%, at least 91%, at least 92%, at least 93%, at least 94%, at least 95%, at least 96%, at least 97%, at least 98%, at least 99%, or even 100% identity” to a specified reference sequence.

Specifically bind: When referring to an antibody or antigen binding fragment, refers to a binding reaction which determines the presence of a target protein in the presence of a heterogeneous population of proteins and other biologics. Thus, under designated conditions, an antibody binds preferentially to a particular target protein, peptide or polysaccharide (such as an antigen present on the surface of a pathogen, for example MPV F protein) and does not bind in a significant amount to other proteins present in the sample or subject. Specific binding can be determined by standard methods. See Harlow & Lane, Antibodies, A Laboratory Manual, 2^{nd} ed., Cold Spring Harbor Publications, New York (2013), for a description of immunoassay formats and conditions that can be used to determine specific immunoreactivity.

With reference to an antibody-antigen complex, specific binding of the antigen and antibody has a K_{D} of less than about 10⁻⁷ Molar, such as less than about 10⁻⁸ Molar, 10⁻⁹, or even less than about 10-10 Molar. K_{D} refers to the dissociation constant for a given interaction, such as a polypeptide ligand interaction or an antibody antigen interaction. For example, for the bimolecular interaction of an antibody or antigen binding fragment and an antigen it is the concentration of the individual components of the bimolecular interaction divided by the concentration of the complex.

An antibody that specifically binds to an epitope on hMPV F protein is an antibody that binds substantially to hMPV F protein, including cells or tissue expressing the hMPV F protein, substrate to which the hMPV F protein is attached, or hMPV F protein in a biological specimen. It is, of course, recognized that a certain degree of non-specific interaction may occur between an antibody and a non-target (such as a cell that does not express hMPV F protein). Typically, specific binding results in a much stronger association between the antibody and protein or cells bearing the antigen than between the antibody and protein or cells lacking the antigen. Specific binding typically results in greater than 2-fold, such as greater than 5-fold, greater than 10-fold, or greater than 100-fold increase in amount of bound antibody (per unit time) to a protein including the epitope or cell or tissue expressing the target epitope as compared to a protein or cell or tissue lacking this epitope. Specific binding to a protein under such conditions requires an antibody that is selected for its specificity for a particular protein. A variety of immunoassay formats are appropriate for selecting antibodies or other ligands specifically immunoreactive with a particular protein. For example, solid-phase ELISA immunoassays are routinely used to select monoclonal antibodies specifically immunoreactive with a protein.

Subject: Living multi-cellular vertebrate organisms, a category that includes human and non-human mammals. In an example, a subject is a human. In a particular example, the subject is a newborn infant. In an additional example, a subject is selected that is in need of inhibiting of an hMPV infection. For example, the subject is either uninfected and at risk of hMPV infection or is infected in need of treatment.

Therapeutically effective amount: The amount of agent, such as a disclosed antibodies or antigen binding fragments thereof that is sufficient to prevent, treat (including prophylaxis), reduce and/or ameliorate the symptoms and/or underlying causes of a disorder or disease, for example to prevent, inhibit, and/or treat an hMPV infection. In some embodiments, a therapeutically effective amount is sufficient to reduce or eliminate a symptom of a disease, such as an hMPV infection. For instance, this can be the amount necessary to inhibit or prevent viral replication or to measurably alter outward symptoms of the viral infection. In general, this amount will be sufficient to measurably inhibit virus replication or infectivity.

In one example, a desired response is to inhibit or reduce or prevent an hMPV infection. The MPV infection does not need to be completely eliminated or reduced or prevented for the method to be effective. For example, administration of a therapeutically effective amount of the agent can decrease the hMPV infection (for example, as measured by infection of cells, or by number or percentage of subjects infected by hMPV) by a desired amount, for example by at least 10%, at least 20%, at least 50%, at least 60%, at least 70%, at least 80%, at least 90%, at least 95%, at least 98%, or even at least 100% (elimination or prevention of detectable hMPV infection, as compared to a suitable control.

A therapeutically effective amount of an agent can be administered in a single dose, or in several doses, for example daily, during a course of treatment (such as a prime-boost vaccination treatment). However, the therapeutically effective amount can depend on the subject being treated, the severity and type of the condition being treated, and the manner of administration. A unit dosage form of the agent can be packaged in a therapeutic amount, or in multiples of the therapeutic amount, for example, in a vial (e.g., with a pierceable lid) or syringe having sterile components.

Treating or preventing a disease: Inhibiting the full development of a disease or condition, for example, in a subject who is at risk of or has a disease such as an hMPV infection. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

The term “reduces” is a relative term, such that an agent reduces a disease or condition if the disease or condition is quantitatively diminished following administration of the agent, or if it is diminished following administration of the agent, as compared to a reference agent. Similarly, the term “prevents” does not necessarily mean that an agent completely eliminates the disease or condition, so long as at least one characteristic of the disease or condition is eliminated. Thus, a composition that reduces or prevents an infection, can, but does not necessarily completely, eliminate such an infection, so long as the infection is measurably diminished, for example, by at least about 50%, such as by at least about 70%, or about 80%, or even by about 90% the infection in the absence of the agent, or in comparison to a reference agent.

Transformed: A transformed cell is a cell into which a nucleic acid molecule has been introduced by molecular biology techniques. As used herein, the term transformed and the like (e.g., transformation, transfection, transduction, etc.) encompasses all techniques by which a nucleic acid molecule might be introduced into such a cell, including transduction with viral vectors, transformation with plasmid vectors, and introduction of DNA by electroporation, lipofection, and particle gun acceleration.

Vector: An entity containing a nucleic acid molecule (such as a DNA or RNA molecule) bearing a promoter(s) that is operationally linked to the coding sequence of a protein of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements. Viral vectors are recombinant nucleic acid vectors having at least some nucleic acid sequences derived from one or more viruses. In some embodiments, a viral vector comprises a nucleic acid molecule encoding a disclosed antibody or antigen binding fragment that specifically binds to hMPV F protein and neutralizes the hMPV.

Under conditions sufficient for: A phrase that is used to describe any environment that permits a desired activity.

### II. Description of Several Embodiments

Isolated monoclonal antibodies and antigen binding fragments that specifically bind hMPV F protein are provided. The antibodies and antigen binding fragments can be fully human. The antibodies and antigen binding fragments can neutralize hMPV, example the disclosed antibodies can inhibit an hMPV infection in vivo, and can be administered prior to, or after, an infection with hMPV. Also disclosed herein are compositions comprising the antibodies and antigen binding fragments and a pharmaceutically acceptable carrier. Nucleic acids encoding the antibodies or antigen binding fragments, expression vectors (such as adeno-associated virus (AAV) viral vectors) comprising these nucleic acids are also provided. The antibodies, antigen binding fragments, nucleic acid molecules, host cells, and compositions can be used for research, diagnostic, treatment and prophylactic purposes. For example, the disclosed antibodies and antigen binding fragments can be used to diagnose a subject with an hMPV infection or can be administered to inhibit an hMPV infection in a subject.

In some embodiments, the disclosed antibodies do not cross react with the RSV F protein. In other embodiments, these antibodies neutralize both genotypes A and B of hMPV. In further embodiments, the disclosed antibodies bind to site III of MPV F protein. In yet other embodiments, the disclosed antibodies bind pre-fusion F protein with a higher affinity than post-fusion F protein.

A. Monoclonal Antibodies that specifically bind hMPV F protein and Antigen Binding Fragments Thereof

The discussion of monoclonal antibodies below refers to isolated monoclonal antibodies that include heavy and/or light chain variable domains (or antigen binding fragments thereof) comprising a CDR1, CDR2, and/or CDR3 with reference to the IMGT numbering scheme (unless the context indicates otherwise). Various CDR numbering schemes (such as the Kabat, Chothia or IMGT numbering schemes) can be used to determine CDR positions. The amino acid sequence and the CDRs of the heavy and light chain of the disclosed monoclonal antibody according to the IMGT numbering scheme are provided in the listing of sequences, but these are exemplary only.

In some embodiments, a monoclonal antibody is provided that comprises the heavy and light chain CDRs of any one of the antibodies described herein. In some embodiment, a monoclonal antibody is provided that comprises the heavy and light chain variable regions of any one of the antibodies described herein.

TABLE 1
IMGT CDRs of Antibodies and SEQ ID NOS
MPV86 V_{H}
QVQLVQSGAEVKKPGSSVKVSCKASGDTFNDVALSWVRQAPGQGLEWLGGIIPFFGTANY
AQRFQDRVTITADASTSTAYLELGGLRSEDTAVYYCARGWSCRNITCFMMGYYFYIMDVW
GQGTLVTVSS (SEQ ID NO: 1)

| | SEQ ID NO: 1 | | CDR |
|---|---|---|---|
| V_{H} | positions | CDR protein sequence | SEQ ID NO |
| HCDR1 | 26-33 | GDTFNDVA | 2 |
| HCDR2 | 51-58 | IIPFFGTA | 3 |
| HCDR3 | 97-119 | ARGWSCRNITCFMMGYYFYI | 4 |
| | | MDV | |

MPV86 Vₖ
LSLSPGERATLSCRASQSVGNFLAWYQQKPGQTPRLLIQDASNRATGIPARFSGTGSGTDFTL
TISSLEPEDFAVYYCQQRSNWPPTFGPGTKVDIK (SEQ ID NO: 5)

| | SEQ ID NO: 5 | | CDR |
|---|---|---|---|
| V_{L} | positions | CDR Sequence | SEQ ID NO |
| LCDR1 | 17-22 | QSVGNF | 6 |
| LCDR2 | 40-42 | DAS | 7 |
| LCDR3 | 79-87 | QQRSNWPPT | 8 |

MPV414 V_{H}
QVQLVQSGPEVKKPGSSVMVSCKTSGGNFNNFAISWVRQAPGQGLEWMGGIVPIFGTANY
AQKSQDRVTITADPSSSTAYLQLSSLTSDDTAIYYCARDLGRSSNYYYYSVFESWGQGTLVT
VSS (SEQ ID NO: 9)

| | SEQ ID NO: 9 | | CDR |
|---|---|---|---|
| V_{H} | positions | CDR protein sequence | SEQ ID NO |
| HCDR1 | 26-33 | GGNFNNFA | 10 |
| HCDR2 | 51-58 | IVPIFGTA | 11 |
| HCDR3 | 97-114 | ARDLGRSSNYYYYSVFES | 12 |

MPV414 Vₖ
DIVMTQSPATLSLSPGERATLSCRASQSVGDKLAWYQQKPGQAPRLLIYGASTRATDIPARF
SGSGSGTEFTLTVSSLQPEDFAVYYCQQYENWPPITFGQGTRLEIK (SEQ ID NO: 13)

| | SEQ ID NO: 13 | | CDR |
|---|---|---|---|
| V_{L} | positions | CDR Sequence | SEQ ID NO |
| LCDR1 | 27-32 | QSVGDK | 14 |
| LCDR2 | 50-52 | GAS | 15 |
| LCDR3 | 89-98 | QQYENWPPIT | 16 |

MPV454 V_{H}
EVQLVESGGGLVKPGGSLRLSCSTSGFVFSSYAMHWVRQAPGKGLEHVSAISSTGDNTHYA
DSVKGRFTISRDNSRGRLYLQMTSVRPEDAALYYCVKDLYSGRFYYFLDDWGQGTRIIVSS
(SEQ ID NO: 17)

| | SEQ ID NO: 17 | | CDR |
|---|---|---|---|
| V_{H} | positions | CDR protein sequence | SEQ ID NO |
| HCDR1 | 26-33 | GFVFSSYA | 18 |
| HCDR2 | 51-58 | ISSTGDNT | 19 |
| HCDR3 | 97-111 | VKDLYSGRFYYFLDD | 20 |

MPV454 Vₖ
DIQVTQSPSSLSASVGDRVTISCRASQNISNYLNWFQQRPGKAPKLLIYVASTLQSGAPSRFS
GSGSGTDFTLTITDLQPEDFATYYCQQSYNTPPITFGQGTRLEIK (SEQ ID NO: 21)

| | SEQ ID NO: 21 | | CDR |
|---|---|---|---|
| V_{L} | positions | CDR Sequence | SEQ ID NO |
| LCDR1 | 27-32 | QNISNY | 22 |
| LCDR2 | 50-52 | VAS | 23 |
| LCDR3 | 89-98 | QQSYNTPPIT | 24 |

MPV456 V_{H}
QVQLQESGPGVVKPSGTLSLTCGVSGGSISSSYWWSWVRQPPGKGLEWIGEIYHSGSTNYN
PSLKRRVTISVDKSKNQFSLKVTSVTAADTAVYYCARGPIQYYDILTEERVGYCDYWGQGT
LVTVSS (SEQ ID NO: 25)

| | SEQ ID NO: 25 | | CDR |
|---|---|---|---|
| V_{H} | positions | CDR protein sequence | SEQ ID NO |
| HCDR1 | 26-34 | GGSISSSYW | 26 |
| HCDR2 | 52-58 | IYHSGST | 27 |
| HCDR3 | 97-117 | ARGPIQYYDILTEERVGYCD | 28 |
| | | Y | |

MPV456 Vₖ
TLSASVGDRVTITCRASQSISSWLAWFQQKPGKAPKLLIYKTSSLESGVPSRFSGSGSGTEFTL
TISSLQPDDFATYYCQEYNSYSRTFGQGTKVEIK (SEQ ID NO: 29)

| | SEQ ID NO: 29 | | CDR |
|---|---|---|---|
| VL | positions | CDR protein sequence | SEQ ID NO |
| LCDR1 | 18-23 | QSISSW | 30 |
| LCDR2 | 41-43 | KTS | 31 |
| LCDR3 | 80-88 | QEYNSYSRT | 32 |

MPV464 V_{H}
QVQLQESGPGLVKPSQTLSLTCTVSGGSVRGGGDYWSWIRRPPGKGLEWIGHIYNSGTTFY
NPSLKSRVTISIDTSKNQFSLKLRSVTAADTAVYYCGRVGSRATGLPDWIDPWGQGTLVTVS
S (SEQ ID NO: 33)

| | SEQ ID NO: 33 | | CDR |
|---|---|---|---|
| V_{H} | positions | CDR protein sequence | SEQ ID NO |
| HCDR1 | 26-35 | GGSVRGGGDY | 34 |
| HCDR2 | 53-59 | IYNSGTT | 35 |
| HCDR3 | 98-113 | GRVGSRATGLPDWIDP | 36 |

MPV464 V_{L}
SYELTQPPSVSVSPGQTATITCSGTNLGAKYSCWYQQKPGQSPVLVIFQDNKRASGIPARFSA
STSGDTATLTISGTQVMDEADYFCQAWDSRTAVFGGGTTLTVL (SEQ ID NO: 37)

| | SEQ ID NO: 37 | | CDR |
|---|---|---|---|
| V_{L} | positions | CDR Sequence | SEQ ID NO |
| LCDR1 | 26-31 | NLGAKY | 38 |
| LCDR2 | 49-51 | QDN | 39 |
| LCDR3 | 88-96 | QAWDSRTAV | 40 |

MPV467 V_{H}
EVQLVESGGGVVRPGTSLRVSCAAFDFNFRDYGMHWVRQAPGKGLEWVAGIWYDGSNKD
YADSVKGRFTISRDNSQNTLYLQMNSLRVEDTAVYYCARDPRTHREGALSHFDSWGQGTL
VTVSS (SEQ ID NO: 41)

| | SEQ ID NO: 41 | | CDR |
|---|---|---|---|
| V_{H} | positions | CDR Sequence | SEQ ID NO |
| HCDR1 | 26-33 | DFNFRDYG | 42 |
| HCDR2 | 51-58 | IWYDGSNK | 43 |
| HCDR3 | 97-113 | ARDPRTHREGALSHEDS | 44 |

MPV467 V_{L}
PNELTQDPAVSVALGQTVRITCQGDSLRNYFAGWYQQKPGQAPLLVLYGENIRPSGIPDRFS
GSSSGNTVSLTITGAQAEDEADYYCNSRDNSGNHWVFGGGTRLTVL (SEQ ID NO: 45)

| | SEQ ID NO: 45 | | CDR |
|---|---|---|---|
| V_{L} | positions | CDR protein sequence | SEQ ID NO |
| LCDR1 | 26-31 | SLRNYF | 46 |
| LCDR2 | 49-51 | GEN | 47 |
| LCDR3 | 88-98 | NSRDNSGNHWV | 48 |

MPV477 V_{H}
EVQLVQSGAEVKKPGASVEVSCKTSGYTFTDYYLHWVRQAPGQGLEWMGWINPRSGGTK
YGQKFQGRVTMTRDTSISTAYMDLRGLRSDDTAVYYCARAPLLTVYAVAHRSGENRFDPW
GQGTVTVSS (SEQ ID NO: 49)

| | SEQ ID NO: 49 | | CDR |
|---|---|---|---|
| V_{H} | positions | CDR Sequence | SEQ ID NO |
| HCDR1 | 26-33 | GYTFTDYY | 50 |
| HCDR2 | 51-58 | INPRSGGT | 51 |
| HCDR3 | 97-118 | ARAPLLTVYAVAHRSGENRF | 52 |
| | | DP | |

MPV477 V_{L}
QPPSLSGAPGQTVTISCTGSGSNIGAGYDVNWYQCLPGTAPKLLMFDNSNRPSGVPDRFSGS
RSGASASLAITGLQAEDEADYYCQSYDSGLSGWVFGGGTKLTVL (SEQ ID NO: 53)

| | SEQ ID NO: 53 | | CDR |
|---|---|---|---|
| V_{H} | positions | CDR protein sequence | SEQ ID NO |
| LCDR1 | 21-29 | GSNIGAGYD | 54 |
| LCDR2 | 47-49 | DNS | 55 |
| LCDR3 | 86-96 | QSYDSGLSGWV | 56 |

MPV478 V_{H}
EVQLVQSGAEVKKPGASVKVSCNASGYTFTGYYIHWVRQAPGQGLEWMGWINPRSGGTN
YAQKFQGRVTLTRDTSITTAYMDVTRLRPDDTAVYYCATTREGIVLMPRGRGDDALDTWG
QGTTVIVSS (SEQ ID NO: 57)

| | SEQ ID NO: 57 | | CDR |
|---|---|---|---|
| V_{H} | positions | CDR Sequence | SEQ ID NO |
| HCDR1 | 26-33 | GYTFTGYY | 58 |
| HCDR2 | 51-58 | INPRSGGT | 59 |
| HCDR3 | 97-117 | ATTREGIVLMPRGRGDDALD | 60 |
| | | T | |

MPV478 Vₖ
DIVMTQSPVSLTVSLGERATINCKSSQSVLSTSNNENYLAWYQQKPGQPPNLLIYWASTRES
GVPDRFSGSGSGTDFTLTISSLQAEDVAVYYCQQYFATPITFGPGTKVDVK (SEQ ID NO: 61)

| | SEQ ID NO: 61 | | CDR |
|---|---|---|---|
| V_{L} | positions | CDR protein sequence | SEQ ID NO |
| LCDR1 | 27-38 | QSVLSTSNNENY | 62 |
| LCDR2 | 56-58 | WAS | 63 |
| LCDR3 | 95-103 | QQYFATPIT | 64 |

MPV481 V_{H}
EVQLVQSGGGLVKPGGSLRLSCAASGLPFNNAWMNWVRQAPGKGLEWVGRIKSKIDGGTT
EYAAPVKGRFTISRDDSENTLYLQMNSLRTEDTAVYYCTTEYYALLSGNYSDCWGQGTLV
TVSS (SEQ ID NO:65)

| | SEQ ID NO: 65 | | CDR |
|---|---|---|---|
| V_{H} | positions | CDR Sequence | SEQ ID NO |
| HCDR1 | 26-33 | GLPFNNAW | 66 |
| HCDR2 | 51-60 | IKSKIDGGTT | 67 |
| HCDR3 | 99-113 | TTEYYALLSGNYSDC | 68 |

MPV481 Vₖ
DIVMTQSPFSLSLSPGERATLSCRASQSIKRAYLGWYQQKPGQAPRLLIYGASNRATGIPDRF
SGSGSGTDFTLTISRLEPEDFAVYYCQQYGDSPGGSFGQGTKLEIR (SEQ ID NO: 69)

| | SEQ ID NO: 69 | | CDR |
|---|---|---|---|
| V_{L} | positions | CDR protein sequence | SEQ ID NO |
| LCDR1 | 27-33 | QSIKRAY | 70 |
| LCDR2 | 51-53 | GAS | 71 |
| LCDR3 | 90-99 | QQYGDSPGGS | 72 |

MPV482 V_{H}
EVQLVQSGGGVVQPGRSLRLSCAASGFTFSSFAMHWVRQAPGKGLEWVALISDDGNNKYH
ADSVKGRFTISRDSSKNTLYLQMNSLRGEDTAVYYCARDGSVVGAAAHCDHWGQGTLVT
VSS (SEQ ID NO: 73)

| | SEQ ID NO: 73 | | CDR |
|---|---|---|---|
| V_{H} | positions | CDR protein sequence | SEQ ID NO |
| HCDR1 | 26-33 | GFTFSSFA | 74 |
| HCDR2 | 51-58 | ISDDGNNK | 75 |
| HCDR3 | 97-111 | ARDGSVVGAAAHCDH | 76 |

MPV482 V_{L}
SYELTQPPSVSVSPGQTARITCSGDALPNQCAYWYQQKPGLAPVLLIYKDKERPSGIPERFSG
SISGTTVTLTISGVQAEDEADYYCQSSDSSSTVVFGGGTKLTVL(SEQ ID NO: 77)

| | SEQ ID NO: 77 | | CDR |
|---|---|---|---|
| V_{L} | positions | CDR Sequence | SEQ ID NO |
| LCDR1 | 26-31 | ALPNQC | 78 |
| LCDR2 | 49-51 | KDK | 79 |
| LCDR3 | 88-97 | QSSDSSSTVV | 80 |

MPV483 V_{H}
EVQLVQSGAELKKPGSSVKVSCKASGDTFSNYAISWVRQAPGQGLEWMGGIIPIYNTANYA
QKFQGRVTLTADESTSTAYMELSSLRSEDTAVYYCARDVRNNWSVLRGARYYYYGMDVW
GQGTTVTVSS (SEQ ID NO: 81)

| | SEQ ID NO: 81 | | CDR |
|---|---|---|---|
| V_{H} | positions | CDR protein sequence | SEQ ID NO |
| HCDR1 | 26-33 | GDTFSNYA | 82 |
| HCDR2 | 51-58 | IIPIYNTA | 83 |
| HCDR3 | 97-119 | ARDVRNNWSVLRGARYYYY | 84 |
| | | GMDV | |

MPV483 Vₖ
DIVMTQTPFSLSASVGDRVTVTCRASQDISNSLAWFQQKPGKAPKSLIYAASSLQSGVPTRFS
GGGSGTDFTLTISSLQPEDFATFYCLQYDSFPPTFGGGTKVEIK (SEQ ID NO: 85)

| | SEQ ID NO: 85 | | CDR |
|---|---|---|---|
| V_{L} | positions | CDR Sequence | SEQ ID NO |
| LCDR1 | 27-32 | QDISNS | 86 |
| LCDR2 | 50-52 | AAS | 87 |
| LCDR3 | 89-97 | LQYDSFPPT | 88 |

MPV485 V_{H}
EVQLQEPGPGLVKPSETLSLTCTVSGGSISSTNSFWGWVRQPPGKGLEWIGSIYYSGTTYYNS
SLKSRVTISVDTSKNQFSLRLSSVTAADTAVYYCARQGLTSSWYDGSGLDVWGRGTKVTVS
S (SEQ ID NO: 89)

| | SEQ ID NO: 89 | | CDR |
|---|---|---|---|
| V_{H} | positions | CDR protein sequence | SEQ ID NO |
| HCDR1 | 26-35 | GGSISSTNSF | 90 |
| HCDR2 | 53-59 | IYYSGTT | 91 |
| HCDR3 | 98-114 | ARQGLTSSWYDGSGLDV | 92 |

MPV485 V_{L}
SYELTQPPSVSVSPGQTASITCSGDKLGNKYACWYQQRPGQSPVLVIYQDTKRPSGIPERFSG
SNSGNTATLTISGTQAMDEADYYCQAWDSNTVVFGGGTKLTVL (SEQ ID NO: 93)

| | SEQ ID NO: 93 | | CDR |
|---|---|---|---|
| V_{L} | positions | CDR Sequence | SEQ ID NO |
| LCDR1 | 26-31 | KLGNKY | 94 |
| LCDR2 | 49-51 | QDT | 95 |
| LCDR3 | 88-96 | QAWDSNTVV | 96 |

MPV486 V_{H}
EVQLVESGGHLVQSGGSLRLSCAASGFTFSNYILNWVRQAPGKGLEWISYITGGSSAIYYAD
SVKGRFTISRDDAKNSLYLQMNNLRAEDTAVYYCARSHGYDSSGYYYYFAMDVWGQGTT
VTVSS (SEQ ID NO: 97)

| | SEQ ID NO: 97 | | CDR |
|---|---|---|---|
| V_{H} | positions | CDR protein sequence | SEQ ID NO |
| HCDR1 | 26-33 | GFTFSNYI | 98 |
| HCDR2 | 51-58 | ITGGSSAI | 99 |
| HCDR3 | 97-115 | ARSHGYDSSGYYYYFAMDV | 100 |

MPV486 V_{L}
QSVLTQPPSASGTPGQRVTISCSGSNSNIESNTVNWYQQIPGTAPKLLIYSDNRRPSGVPDRFS
GSKSGTSASLAISGLQSEDEADYYCAAWDDSLIGYVFGTGTKVTV (SEQ ID NO: 101)

| | SEQ ID NO: 101 | | CDR |
|---|---|---|---|
| V_{L} | positions | CDR Sequence | SEQ ID NO |
| LCDR1 | 26-33 | NSNIESNT | 102 |
| LCDR2 | 51-53 | SDN | 103 |
| LCDR3 | 90-100 | AAWDDSLIGYV | 104 |

MPV487 V_{H}
EVQLLESGGGLVQPGRSLRLSCTASGFTFDDYAMHWVRQLPGKGLEWVSGISWNSDNIGY
ADSVKGRFTISRDNGKNSLYLQMNSLRAEDTAFYYCAKDVRIDYDLLIGHIDYWGPGTLVT
VSS (SEQ ID NO: 105)

| | SEQ ID NO: 105 | | CDR |
|---|---|---|---|
| V_{H} | positions | CDR protein sequence | SEQ ID NO |
| HCDR1 | 26-33 | GFTFDDYA | 106 |
| HCDR2 | 51-58 | ISWNSDNI | 107 |
| HCDR3 | 97-113 | AKDVRIDYDLLIGHIDY | 108 |

MPV487 Vₖ
TQSPVTLSLSPGERATLSCRASQSFGGYCAWYQQKPGQSPRLLIYDASNRATGVPARFSGSG
SGTDFTLTISSLEPEDSAVYFCQQRGNWPIFGQGTRLEIK (SEQ ID NO: 109)

| | SEQ ID NO: 109 | | CDR |
|---|---|---|---|
| V_{L} | positions | CDR Sequence | SEQ ID NO |
| LCDR1 | 23-28 | QSFGGY | 110 |
| LCDR2 | 46-48 | DAS | 111 |
| LCDR3 | 85-92 | QQRGNWPI | 112 |

MPV488 V_{H}
EVQLVQSGFEVKKPGASVKVSCKASGYNFNNYGISWVRQAPGQGLEWMGWISAYTGNTN
EAQKFQGRVSMTTDTSTSTAYMEVRSLRSDDTAVYYCARDIGSSMFYSYFYGMDVWGQG
TTVIVSS (SEQ ID NO: 113)

| | SEQ ID NO: 113 | | CDR |
|---|---|---|---|
| V_{H} | positions | CDR protein sequence | SEQ ID NO |
| HCDR1 | 26-33 | GYNFNNYG | 114 |
| HCDR2 | 51-58 | ISAYTGNT | 115 |
| HCDR3 | 97-114 | ARDIGSSMFYSYFYGMDV | 116 |

MPV488 V_{K}
SLSVSLGERATINCKSSESVLYNSNNENYLDWYQQKPGQPPKLLIYWASTRASGVPDRESGS
ASGTDFTLTISSLQAEDVAVYYCQQYYSTPRTFGQGTKVEIK (SEQ ID NO: 117)

| | SEQ ID NO: 117 | | CDR |
|---|---|---|---|
| V_{L} | positions | CDR Sequence | SEQ ID NO |
| LCDR1 | 18-29 | ESVLYNSNNENY | 118 |
| LCDR2 | 47-49 | WAS | 119 |
| LCDR3 | 86-94 | QQYYSTPRT | 120 |

MPV489 V_{H}
EVQLLESGGGLVQPGGSLRLSCAASKFTFNNYEMNWLRQAPGKGLEWVSSISSSGDTIHNA
DSVKGRFIISRDNAKNSLHLQMNGLRAEDTAVYYCARAGSGWAYDAFDIWGLGTMVTVSS
(SEQ ID NO: 121)

| | SEQ ID NO: 121 | | CDR |
|---|---|---|---|
| V_{H} | positions | CDR protein sequence | SEQ ID NO |
| HCDR1 | 26-33 | KFTFNNYE | 122 |
| HCDR2 | 51-58 | ISSSGDTI | 123 |
| HCDR3 | 97-110 | ARAGSGWAYDAFDI | 124 |

MPV489 V_{K}
LSASVGDRVTITCRASQSISSFLNWYQQKPGKAPKLLMYAASSLPVGVPSRFSGSGSGPEYT
LTISNLQPEDFATYYCQQGYSSPPTFGGGTRLEVK (SEQ ID NO: 125)

| | SEQ ID NO: 125 | | CDR |
|---|---|---|---|
| V_{L} | positions | CDR Sequence | SEQ ID NO |
| LCDR1 | 17-22 | QSISSF | 126 |
| LCDR2 | 40-42 | AAS | 127 |
| LCDR3 | 79-87 | QQGYSSPPT | 128 |

MPV491 V_{H}
QVQLQESGPGLVKSSQTLSLTCTVSGGSISSVTSHWTWIRQHPGKGLEWIGYIFSSGTTYYSP
SLRSRLTMSVDTSKNQFSLQLSSVTAADTAMYYCARGIYCGDSCYKGTDYWGRGTLVTVS
S (SEQ ID NO: 129)

| | SEQ ID NO: 129 | | CDR |
|---|---|---|---|
| VH | positions | CDR protein sequence | SEQ ID NO |
| HCDR1 | 26-31 | GGSISS | 130 |
| HCDR2 | 53-59 | IFSSGTT | 131 |
| HCDR3 | 98-113 | ARGIYCGDSCYKGTDY | 132 |

MPV491 Vₖ
IRLMTQTPVTLPVTPGEPASISCRSSQSLLHSNGYNYLDWYLQKPGQSPQLLIYRGSTRASGV
PDRFSGSGSGTEFTLKISRVEAEDVGVYYCMQGLQTPYTFGQGTKLESR (SEQ ID NO: 133)

| | SEQ ID NO: 133 | | CDR |
|---|---|---|---|
| V_{L} | positions | CDR Sequence | SEQ ID NO |
| LCDR1 | 27-37 | QSLLHSNGYNY | 134 |
| LCDR2 | 55-57 | RGS | 135 |
| LCDR3 | 94-102 | MQGLQTPYT | 136 |

MPV503 V_{H}
EVQLVQSGAEVKKPGSSVKVSCKASGGTFSSYTFSWVRQAPGQGLEWMGGIIPIFGTANYA
QKFQDRVTITADESTSTAYMELSSLRSEDTAVYYCARGRVVVIASHYYYYDMDVWGQGTT
VTVSS (SEQ ID NO: 137)

| | SEQ ID NO: 137 | | CDR |
|---|---|---|---|
| V_{H} | positions | CDR protein sequence | SEQ ID NO |
| HCDR1 | 26-33 | GGTFSSYT | 138 |
| HCDR2 | 51-58 | IIPIFGTA | 139 |
| HCDR3 | 97-115 | ARGRVVVIASHYYYYDMDV | 140 |

MPV503 Vₖ
VSLSVSPGERATLSCRASQSVSSNLAWYQQKPGQAPRLLIYGASTRATGIPARFSGSGSGTEF
TLTISSLQSEDFAVYYCQQYDNWPPLTFGGGTKVEIK (SEQ ID NO: 141)

| | SEQ ID NO: 141 | | CDR |
|---|---|---|---|
| V_{L} | positions | CDR Sequence | SEQ ID NO |
| LCDR1 | 19-24 | QSVSSN | 142 |
| LCDR2 | 42-44 | GAS | 143 |
| LCDR3 | 81-90 | QQYDNWPPLT | 144 |

a. Monoclonal Antibody MPV86

In some embodiments, the antibody or antigen binding fragment is based on or derived from the MPV86 antibody, and specifically binds to hMPV F protein and neutralizes hMPV.

In some examples, the antibody or antigen binding fragment comprises a V_{H} and a V_{L} Comprising the HCDR1, the HCDR2, the H-CDR3, the LCDR1, the LCDR2, and the LCDR3, respectively (for example, according to IMGT, Kabat or Chothia), of the MPV86 antibody, and specifically binds to hMPV F protein and neutralizes hMPV.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 1, and specifically binds to binds to hMPV F protein and neutralizes hMPV. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 5, and specifically binds to hMPV F protein and neutralizes hMPV. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 1 and 5, respectively, and specifically binds to binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 2, 3, and 4, respectively, and/or a V_{L} comprising a LCDR1, a LCDR2, and a LCDR3 as set forth as SEQ ID NOs: 6, 7, and 8, respectively, and specifically binds to hMPV F protein and neutralizes hMPV In some embodiments, the antibody or antigen binding fragment comprises a V_{H} comprising a HCDR1, a HCDR2, and a HCDR3 as set forth as SEQ ID NOs: 2, 3, and 4, respectively, a V_{L} comprising a LCDR1, a LCDR2, and a LCDR3 as set forth as SEQ ID NOs: 6, 7, and 8 respectively, wherein the V_{H} comprises an amino acid sequence at least 90% identical to SEQ ID NO: 1, such as 95%, 96%, 97%, 98% or 99% identical to SEQ ID NO: 1, and wherein the V_{L} comprises an amino acid sequence at least 90% identical to SEQ ID NO: 5, such as 95%, 96%, 97%, 98% or 99% identical to SEQ ID NO: 5, and the antibody or antigen binding fragment specifically binds to hMPV F protein and neutralizes hMPV. In this embodiment, variations due to sequence identify fall outside the CDRs.

In some embodiments, the antibody or antigen binding fragment comprises a V_{H} comprising the amino acid sequence set forth as SEQ ID NO: 1, and specifically binds to hMPV F protein and neutralizes hMPV. In more embodiments, the antibody or antigen binding fragment comprises a V_{L} comprising the amino acid sequence set forth as SEQ ID NO: 5, and specifically binds to hMPV F protein and neutralizes hMPV. In some embodiments, the antibody or antigen binding fragment comprises a V_{H} and a V_{L} comprising the amino acid sequences set forth as SEQ ID NOs: 1 and 5, respectively, and specifically binds to hMPV F protein and neutralizes hMPV.

b. Monoclonal Antibody MPV414

In some embodiments, the antibody or antigen binding fragment is based on or derived from the MPV414 antibody, and specifically binds to hMPV F protein and neutralizes hMPV. In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the MPV414 antibody, and specifically binds to hMPV F protein and neutralizes hMPV.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 9, and specifically binds to hMPV F protein and neutralizes hMPV. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 13, and specifically binds to hMPV F protein and neutralizes hMPV. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 9 and 13, respectively, and specifically binds to binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 10, 11, and 12, respectively, and/or a V_{L} comprising a LCDR1, a LCDR2, and a LCDR3 as set forth as SEQ ID NOs: 14, 15 and 16, respectively, and specifically binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 10, 11, and 12, respectively, a V_{L} comprising a LCDR1, a LCDR2, and a LCDR3 as set forth as SEQ ID NOs: 14, 15 and 16, respectively, wherein the V_{H} comprises an amino acid sequence at least 90% identical to SEQ ID NO: 9, such as 95%, 96%, 97%, 98% or 99% identical to SEQ ID NO: 9, and wherein the V_{L} comprises an amino acid sequence at least 90% identical to SEQ ID NO: 13, such as 95%, 96%, 97%, 98% or 99% identical to SEQ ID NO: 13, and the antibody or antigen binding fragment specifically binds to hMPV F protein and neutralizes hMPV. In this embodiment, variations due to sequence identify fall outside the CDRs.

In some embodiments, the antibody or antigen binding fragment comprises a V_{H} comprising the amino acid sequence set forth as SEQ ID NO: 9, and specifically binds to hMPV F protein and neutralizes hMPV. In more embodiments, the antibody or antigen binding fragment comprises a V_{L} comprising the amino acid sequence set forth as SEQ ID NO: 13, and specifically binds to hMPV F protein and neutralizes hMPV. In some embodiments, the antibody or antigen binding fragment comprises a V_{H} and a V_{L} comprising the amino acid sequences set forth as SEQ ID NOs: 9 and 13, respectively, and specifically binds to hMPV F protein and neutralizes hMPV.

c. Monoclonal Antibody MPV454

In some embodiments, the antibody or antigen binding fragment is based on or derived from the MPV454 antibody, and specifically binds to hMPV F protein and neutralizes hMPV.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the MPV454 antibody, and specifically binds to hMPV F protein and neutralizes hMPV.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 17, and specifically binds to hMPV F protein and neutralizes hMPV. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 21, and specifically binds to hMPV F protein and neutralizes hMPV. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 17 and 21, respectively, and specifically binds to binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 18, 19, and 20, respectively, and/or a V_{L} comprising a LCDR1, a LCDR2, and a LCDR3 as set forth as SEQ ID NOs: 22, 23, and 24, respectively, and specifically binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 18, 19, and 20, respectively, a V_{L} comprising a LCDR1, a LCDR2, and a LCDR3 as set forth as SEQ ID NOs: 22, 23, and 24, respectively, wherein the V_{H} comprises an amino acid sequence at least 90% identical to SEQ ID NO: 17, such as 95%, 96%, 97%, 98% or 99% identical to SEQ ID NO: 17, and wherein the V_{L} comprises an amino acid sequence at least 90% identical to SEQ ID NO: 21, such as 95%, 96%, 97%, 98% or 99% identical to SEQ ID NO: 21, and the antibody or antigen binding fragment specifically binds to hMPV F protein and neutralizes hMPV. In this embodiment, variations due to sequence identify fall outside the CDRs.

In some embodiments, the antibody or antigen binding fragment comprises a V_{H} comprising the amino acid sequence set forth as SEQ ID NO: 17, and specifically binds to hMPV F protein and neutralizes hMPV. In more embodiments, the antibody or antigen binding fragment comprises a V_{L} comprising the amino acid sequence set forth as SEQ ID NO: 21, and specifically binds to hMPV F protein and neutralizes hMPV. In some embodiments, the antibody or antigen binding fragment comprises a V_{H} and a V_{L} comprising the amino acid sequences set forth as SEQ ID NOs: 17 and 21, respectively, and specifically binds to hMPV F protein and neutralizes hMPV.

d. Monoclonal Antibody MPV456

In some embodiments, the antibody or antigen binding fragment is based on or derived from the MPV456 antibody, and specifically binds to hMPV F protein and neutralizes hMPV.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the MPV456 antibody, and specifically binds to hMPV F protein and neutralizes hMPV.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 25, and specifically binds to hMPV F protein and neutralizes hMPV. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 29, and specifically binds to hMPV F protein and neutralizes hMPV. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 25 and 29, respectively, and binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 26, 27, and 28, respectively, and/or a V_{L}, comprising a LCDR1, a LCDR2, and a LCDR3 as set forth as SEQ ID NOs: 30, 31, and 32, respectively, and specifically binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 26, 27, and 28, respectively, a V_{L} comprising a LCDR1, a LCDR2, and a LCDR3 as set forth as SEQ ID NOs: 30, 31, and 32, respectively, wherein the V_{H} comprises an amino acid sequence at least 90% identical to SEQ ID NO: 25, such as 95%, 96%, 97%, 98% or 99% identical to SEQ ID NO: 25, and wherein the V_{L} comprises an amino acid sequence at least 90% identical to SEQ ID NO: 29, such as 95%, 96%, 97%, 98% or 99% identical to SEQ ID NO: 29, and the antibody or antigen binding fragment specifically binds to hMPV F protein and neutralizes hMPV. In this embodiment, variations due to sequence identify fall outside the CDRs.

In some embodiments, the antibody or antigen binding fragment comprises a V_{H} comprising the amino acid sequence set forth as SEQ ID NO: 25, and specifically binds to hMPV F protein and neutralizes hMPV. In more embodiments, the antibody or antigen binding fragment comprises a V_{L} comprising the amino acid sequence set forth as SEQ ID NO: 29, and specifically binds to hMPV F protein and neutralizes hMPV. In some embodiments, the antibody or antigen binding fragment comprises a V_{H} and a V_{L} comprising the amino acid sequences set forth as SEQ ID NOs: 25 and 29, respectively, and specifically binds to hMPV F protein and neutralizes hMPV.

e. Monoclonal Antibody MPV464

In some embodiments, the antibody or antigen binding fragment is based on or derived from the MPV464 antibody, and specifically binds to hMPV F protein and neutralizes hMPV.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the MPV464 antibody, and specifically binds to hMPV F protein and neutralizes hMPV

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 33, and specifically binds to hMPV F protein and neutralizes hMPV. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 37, and specifically binds to hMPV F protein and neutralizes hMPV. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 33 and 37, respectively, and binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 34, 35, and 36, respectively, and/or a V_{L} comprising a LCDR1, a LCDR2, and a LCDR3 as set forth as SEQ ID NOs: 38, 39, and 40, respectively, and specifically binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 34, 35, and 36, respectively, a V_{L} comprising a LCDR1, a LCDR2, and a LCDR3 as set forth as SEQ ID NOs: 38, 39, and 40, respectively, wherein the V_{H} comprises an amino acid sequence at least 90% identical to SEQ ID NO: 33, such as 95%, 96%, 97%, 98% or 99% identical to SEQ ID NO: 33, and wherein the V_{L} comprises an amino acid sequence at least 90% identical to SEQ ID NO: 37, such as 95%, 96%, 97%, 98% or 99% identical to SEQ ID NO: 37, and the antibody or antigen binding fragment specifically binds to hMPV F protein and neutralizes hMPV. In this embodiment, variations due to sequence identify fall outside the CDRs.

In some embodiments, the antibody or antigen binding fragment comprises a V_{H} comprising the amino acid sequence set forth as SEQ ID NO: 33, and specifically binds to hMPV F protein and neutralizes hMPV. In more embodiments, the antibody or antigen binding fragment comprises a V_{L} comprising the amino acid sequence set forth as SEQ ID NO: 37, and specifically binds to hMPV F protein and neutralizes hMPV. In some embodiments, the antibody or antigen binding fragment comprises a V_{H} and a V_{L} comprising the amino acid sequences set forth as SEQ ID NOs: 33 and 37, respectively, and specifically binds to hMPV F protein and neutralizes hMPV.

f. Monoclonal Antibody MPV467

In some embodiments, the antibody or antigen binding fragment is based on or derived from the MPV467 antibody, and specifically binds to hMPV F protein and neutralizes hMPV.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the MPV467 antibody, and specifically binds to hMPV F protein and neutralizes hMPV.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 41, and specifically binds to hMPV F protein and neutralizes hMPV. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 45, and specifically binds to hMPV F protein and neutralizes hMPV. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 41 and 45, respectively, and binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 42, 43, and 44, respectively, and/or a V_{L} comprising a LCDR1, a LCDR2, and a LCDR3 as set forth as SEQ ID NOs: 46, 47, and 48, respectively, and specifically binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 42, 43, and 44, respectively, a V_{L} comprising a LCDR1, a LCDR2, and a LCDR3 as set forth as SEQ ID NOs: 46, 47, and 48, respectively, wherein the V_{H} comprises an amino acid sequence at least 90% identical to SEQ ID NO: 41, such as 95%, 96%, 97%, 98% or 99% identical to SEQ ID NO: 41, and wherein the V_{L} comprises an amino acid sequence at least 90% identical to SEQ ID NO: 45, such as 95%, 96%, 97%, 98% or 99% identical to SEQ ID NO: 45, and the antibody or antigen binding fragment specifically binds to hMPV F protein and neutralizes hMPV. In this embodiment, variations due to sequence identify fall outside the CDRs.

In some embodiments, the antibody or antigen binding fragment comprises a V_{H} comprising the amino acid sequence set forth as SEQ ID NO: 41, and specifically binds to hMPV F protein and neutralizes hMPV. In more embodiments, the antibody or antigen binding fragment comprises a V_{L} comprising the amino acid sequence set forth as SEQ ID NO: 45, and specifically binds to hMPV F protein and neutralizes hMPV. In some embodiments, the antibody or antigen binding fragment comprises a V_{H} and a V_{L} comprising the amino acid sequences set forth as SEQ ID NOs: 41 and 45, respectively, and specifically binds to hMPV F protein and neutralizes hMPV.

g. Monoclonal Antibody MPV477

In some embodiments, the antibody or antigen binding fragment is based on or derived from the MPV477 antibody, and specifically binds to hMPV F protein and neutralizes hMPV.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the MPV477 antibody, and specifically binds to hMPV F protein and neutralizes hMPV.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 49, and specifically binds to hMPV F protein and neutralizes hMPV. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 53, and specifically binds to hMPV F protein and neutralizes hMPV. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 49 and 53, respectively, and binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 50, 51, and 52, respectively, and/or a V_{L} comprising a LCDR1, a LCDR2, and a LCDR3 as set forth as SEQ ID NOs: 54, 55, and 56, respectively, and specifically binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 50, 51, and 52, respectively, a V_{L} comprising a LCDR1, a LCDR2, and a LCDR3 as set forth as SEQ ID NOs: 54, 55, and 56, respectively, wherein the V_{H} comprises an amino acid sequence at least 90% identical to SEQ ID NO: 49, such as 95%, 96%, 97%, 98% or 99% identical to SEQ ID NO: 49, and wherein the V_{L} comprises an amino acid sequence at least 90% identical to SEQ ID NO: 53, such as 95%, 96%, 97%, 98% or 99% identical to SEQ ID NO: 53, and the antibody or antigen binding fragment specifically binds to hMPV F protein and neutralizes hMPV. In this embodiment, variations due to sequence identify fall outside the CDRs.

In some embodiments, the antibody or antigen binding fragment comprises a V_{H} comprising the amino acid sequence set forth as SEQ ID NO: 49, and specifically binds to hMPV F protein and neutralizes hMPV. In more embodiments, the antibody or antigen binding fragment comprises a V_{L} comprising the amino acid sequence set forth as SEQ ID NO: 53, and specifically binds to hMPV F protein and neutralizes hMPV. In some embodiments, the antibody or antigen binding fragment comprises a V_{H} and a V_{L} comprising the amino acid sequences set forth as SEQ ID NOs: 49 and 53, respectively, and specifically binds to hMPV F protein and neutralizes hMPV.

h. Monoclonal Antibody MPV478

In some embodiments, the antibody or antigen binding fragment is based on or derived from the MPV478 antibody, and specifically binds to hMPV F protein and neutralizes hMPV.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the MPV478 antibody, and specifically binds to hMPV F protein and neutralizes hMPV.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 57, and specifically binds to hMPV F protein and neutralizes hMPV. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 61, and specifically binds to hMPV F protein and neutralizes hMPV. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 57 and 61, respectively, and binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 58, 59, and 60, respectively, and/or a V_{L} comprising a LCDR1, a LCDR2, and a LCDR3 as set forth as SEQ ID NOs: 62, 63, and 64, respectively, and specifically binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 58, 59, and 60, respectively, a V_{L} comprising a LCDR1, a LCDR2, and a LCDR3 as set forth as SEQ ID NOs: 62, 63, and 64, respectively, wherein the V_{H} comprises an amino acid sequence at least 90% identical to SEQ ID NO: 57, such as 95%, 96%, 97%, 98% or 99% identical to SEQ ID NO: 57, and wherein the V_{L} comprises an amino acid sequence at least 90% identical to SEQ ID NO: 61, such as 95%, 96%, 97%, 98% or 99% identical to SEQ ID NO: 61, and the antibody or antigen binding fragment specifically binds to hMPV F protein and neutralizes hMPV. In this embodiment, variations due to sequence identify fall outside the CDRs.

In some embodiments, the antibody or antigen binding fragment comprises a V_{H} comprising the amino acid sequence set forth as SEQ ID NO: 57, and specifically binds to hMPV F protein and neutralizes hMPV. In more embodiments, the antibody or antigen binding fragment comprises a V_{L} comprising the amino acid sequence set forth as SEQ ID NO: 61, and specifically binds to hMPV F protein and neutralizes hMPV. In some embodiments, the antibody or antigen binding fragment comprises a V_{H} and a V_{L} comprising the amino acid sequences set forth as SEQ ID NOs: 57 and 61, respectively, and specifically binds to hMPV F protein and neutralizes hMPV.

i. Monoclonal Antibody MPV481

In some embodiments, the antibody or antigen binding fragment is based on or derived from the MPV481 antibody, and specifically binds to hMPV F protein and neutralizes hMPV.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the MPV481 antibody, and specifically binds to hMPV F protein and neutralizes hMPV.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 65, and specifically binds to hMPV F protein and neutralizes hMPV. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 69, and specifically binds to hMPV F protein and neutralizes hMPV. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 65 and 69, respectively, and binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 66, 67, and 68, respectively, and/or a V_{L} comprising a LCDR1, a LCDR2, and a LCDR3 as set forth as SEQ ID NOs: 70, 71, and 72, respectively, and specifically binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 66, 67, and 68, respectively, a V_{L} comprising a LCDR1, a LCDR2, and a LCDR3 as set forth as SEQ ID NOs: 70, 71, and 72, respectively, wherein the V_{H} comprises an amino acid sequence at least 90% identical to SEQ ID NO: 65, such as 95%, 96%, 97%, 98% or 99% identical to SEQ ID NO: 65, and wherein the V_{L} comprises an amino acid sequence at least 90% identical to SEQ ID NO: 69, such as 95%, 96%, 97%, 98% or 99% identical to SEQ ID NO: 69, and the antibody or antigen binding fragment specifically binds to hMPV F protein and neutralizes hMPV. In this embodiment, variations due to sequence identify fall outside the CDRs.

In some embodiments, the antibody or antigen binding fragment comprises a V_{H} comprising the amino acid sequence set forth as SEQ ID NO: 65, and specifically binds to hMPV F protein and neutralizes hMPV. In more embodiments, the antibody or antigen binding fragment comprises a V_{L} comprising the amino acid sequence set forth as SEQ ID NO: 69, and specifically binds to hMPV F protein and neutralizes hMPV. In some embodiments, the antibody or antigen binding fragment comprises a V_{H} and a V_{L} comprising the amino acid sequences set forth as SEQ ID NOs: 65 and 69, respectively, and specifically binds to hMPV F protein and neutralizes hMPV.

j. Monoclonal Antibody MPV482

In some embodiments, the antibody or antigen binding fragment is based on or derived from the MPV482 antibody, and specifically binds to hMPV F protein and neutralizes hMPV.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the MPV482 antibody, and specifically binds to hMPV F protein and neutralizes hMPV.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 73, and specifically binds to hMPV F protein and neutralizes hMPV. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 77, and specifically binds to hMPV F protein and neutralizes hMPV. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 73 and 77, respectively, and binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 74, 75, and 76, respectively, and/or a V_{L} comprising a LCDR1, a LCDR2, and a LCDR3 as set forth as SEQ ID NOs: 78, 79, and 80, respectively, and specifically binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 74, 75, and 76, respectively, a V_{L} comprising a LCDR1, a LCDR2, and a LCDR3 as set forth as SEQ ID NOs: 78, 79, and 80, respectively, wherein the V_{H} comprises an amino acid sequence at least 90% identical to SEQ ID NO: 73, such as 95%, 96%, 97%, 98% or 99% identical to SEQ ID NO: 73, and wherein the V_{L} comprises an amino acid sequence at least 90% identical to SEQ ID NO: 77, such as 95%, 96%, 97%, 98% or 99% identical to SEQ ID NO: 77, and the antibody or antigen binding fragment specifically binds to hMPV F protein and neutralizes hMPV. In this embodiment, variations due to sequence identify fall outside the CDRs.

In some embodiments, the antibody or antigen binding fragment comprises a V_{H} comprising the amino acid sequence set forth as SEQ ID NO: 73 and specifically binds to hMPV F protein and neutralizes hMPV. In more embodiments, the antibody or antigen binding fragment comprises a V_{L} comprising the amino acid sequence set forth as SEQ ID NO: 77, and specifically binds to hMPV F protein and neutralizes hMPV. In some embodiments, the antibody or antigen binding fragment comprises a V_{H} and a V_{L} comprising the amino acid sequences set forth as SEQ ID NOs: 73 and 77, respectively, and specifically binds to hMPV F protein and neutralizes hMPV.

k. Monoclonal Antibody MPV483

In some embodiments, the antibody or antigen binding fragment is based on or derived from the MPV483 antibody, and specifically binds to hMPV F protein and neutralizes hMPV.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the MPV483 antibody, and specifically binds to hMPV F protein and neutralizes hMPV.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 81, and specifically binds to hMPV F protein and neutralizes hMPV. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 85, and specifically binds to hMPV F protein and neutralizes hMPV. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 81 and 85, respectively, and binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 82, 83, and 84, respectively, and/or a V_{L} comprising a LCDR1, a LCDR2, and a LCDR3 as set forth as SEQ ID NOs: 86, 87, and 88, respectively, and specifically binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 82, 83, and 84, respectively, a V_{L} comprising a LCDR1, a LCDR2, and a LCDR3 as set forth as SEQ ID NOs: 86, 87, and 88, respectively, wherein the V_{H} comprises an amino acid sequence at least 90% identical to SEQ ID NO: 81, such as 95%, 96%, 97%, 98% or 99% identical to SEQ ID NO: 81, and wherein the V_{L} comprises an amino acid sequence at least 90% identical to SEQ ID NO: 85, such as 95%, 96%, 97%, 98% or 99% identical to SEQ ID NO: 85, and the antibody or antigen binding fragment specifically binds to hMPV F protein and neutralizes hMPV. In this embodiment, variations due to sequence identify fall outside the CDRs.

In some embodiments, the antibody or antigen binding fragment comprises a V_{H} comprising the amino acid sequence set forth as SEQ ID NO: 81, and specifically binds to hMPV F protein and neutralizes hMPV. In more embodiments, the antibody or antigen binding fragment comprises a V_{L} comprising the amino acid sequence set forth as SEQ ID NO: 85, and specifically binds to hMPV F protein and neutralizes hMPV. In some embodiments, the antibody or antigen binding fragment comprises a V_{H} and a V_{L} comprising the amino acid sequences set forth as SEQ ID NOs: 81 and 85, respectively, and specifically binds to hMPV F protein and neutralizes hMPV.

l. Monoclonal Antibody MPV485

In some embodiments, the antibody or antigen binding fragment is based on or derived from the MPV485 antibody, and specifically binds to hMPV F protein and neutralizes hMPV.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the MPV485 antibody, and specifically binds to hMPV F protein and neutralizes hMPV.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 89, and specifically binds to hMPV F protein and neutralizes hMPV. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 93, and specifically binds to hMPV F protein and neutralizes hMPV. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 89 and 93, respectively, and binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 90, 91, and 92, respectively, and/or a V_{L} comprising a LCDR1, a LCDR2, and a LCDR3 as set forth as SEQ ID NOs: 94, 95, and 96, respectively, and specifically binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 90, 91, and 92, respectively, a V_{L} comprising a LCDR1, a LCDR2, and a LCDR3 as set forth as SEQ ID NOs: 94, 95, and 96, respectively, wherein the V_{H} comprises an amino acid sequence at least 90% identical to SEQ ID NO: 89, such as 95%, 96%, 97%, 98% or 99% identical to SEQ ID NO: 89, and wherein the V_{L} comprises an amino acid sequence at least 90% identical to SEQ ID NO: 93, such as 95%, 96%, 97%, 98% or 99% identical to SEQ ID NO: 93, and the antibody or antigen binding fragment specifically binds to hMPV F protein and neutralizes hMPV. In this embodiment, variations due to sequence identify fall outside the CDRs.

In some embodiments, the antibody or antigen binding fragment comprises a V_{H} comprising the amino acid sequence set forth as SEQ ID NO: 89, and specifically binds to hMPV F protein and neutralizes hMPV. In more embodiments, the antibody or antigen binding fragment comprises a V_{L} comprising the amino acid sequence set forth as SEQ ID NO: 93, and specifically binds to hMPV F protein and neutralizes hMPV. In some embodiments, the antibody or antigen binding fragment comprises a V_{H} and a V_{L} comprising the amino acid sequences set forth as SEQ ID NOs: 89 and 93, respectively, and specifically binds to hMPV F protein and neutralizes hMPV.

m. Monoclonal Antibody MPV486

In some embodiments, the antibody or antigen binding fragment is based on or derived from the MPV486 antibody, and specifically binds to hMPV F protein and neutralizes hMPV.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the MPV486 antibody, and specifically binds to hMPV F protein and neutralizes hMPV.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 97, and specifically binds to hMPV F protein and neutralizes hMPV. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 101, and specifically binds to hMPV F protein and neutralizes hMPV. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 97 and 101, respectively, and binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 98, 99, and 100, respectively, and/or a V_{L} comprising a LCDR1, a LCDR2, and a LCDR3 as set forth as SEQ ID NOs: 102, 103, and 104, respectively, and specifically binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 98, 99, and 100, respectively, a V_{L} comprising a LCDR1, a LCDR2, and a LCDR3 as set forth as SEQ ID NOs: 102, 103, and 104, respectively, wherein the V_{H} comprises an amino acid sequence at least 90% identical to SEQ ID NO: 97, such as 95%, 96%, 97%, 98% or 99% identical to SEQ ID NO: 97, and wherein the V_{L} comprises an amino acid sequence at least 90% identical to SEQ ID NO: 101, such as 95%, 96%, 97%, 98% or 99% identical to SEQ ID NO: 101, and the antibody or antigen binding fragment specifically binds to hMPV F protein and neutralizes hMPV. In this embodiment, variations due to sequence identify fall outside the CDRs.

In some embodiments, the antibody or antigen binding fragment comprises a V_{H} comprising the amino acid sequence set forth as SEQ ID NO: 97, and specifically binds to hMPV F protein and neutralizes hMPV. In more embodiments, the antibody or antigen binding fragment comprises a V_{L} comprising the amino acid sequence set forth as SEQ ID NO: 101, and specifically binds to hMPV F protein and neutralizes hMPV. In some embodiments, the antibody or antigen binding fragment comprises a V_{H} and a V_{L} comprising the amino acid sequences set forth as SEQ ID NOs: 97 and 101, respectively, and specifically binds to hMPV F protein and neutralizes hMPV.

n. Monoclonal Antibody MPV487

In some embodiments, the antibody or antigen binding fragment is based on or derived from the MPV487 antibody, and specifically binds to hMPV F protein and neutralizes hMPV.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the MPV487 antibody, and specifically binds to hMPV F protein and neutralizes hMPV.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 105, and specifically binds to hMPV F protein and neutralizes hMPV. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 109, and specifically binds to hMPV F protein and neutralizes hMPV. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 105 and 109, respectively, and binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 106, 107, and 108, respectively, and/or a V_{L} comprising a LCDR1, a LCDR2, and a LCDR3 as set forth as SEQ ID NOs: 110, 111, and 112, respectively, and specifically binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 106, 107, and 108, respectively, a V_{L} comprising a LCDR1, a LCDR2, and a LCDR3 as set forth as SEQ ID NOs: 110, 111, and 112, respectively, wherein the V_{H} comprises an amino acid sequence at least 90% identical to SEQ ID NO: 105, such as 95%, 96%, 97%, 98% or 99% identical to SEQ ID NO: 105, and wherein the V_{L} comprises an amino acid sequence at least 90% identical to SEQ ID NO: 109, such as 95%, 96%, 97%, 98% or 99% identical to SEQ ID NO: 109, and the antibody or antigen binding fragment specifically binds to hMPV F protein and neutralizes hMPV. In this embodiment, variations due to sequence identify fall outside the CDRs.

In some embodiments, the antibody or antigen binding fragment comprises a V_{H} comprising the amino acid sequence set forth as SEQ ID NO: 105, and specifically binds to hMPV F protein and neutralizes hMPV. In more embodiments, the antibody or antigen binding fragment comprises a V_{L} comprising the amino acid sequence set forth as SEQ ID NO: 109, and specifically binds to hMPV F protein and neutralizes hMPV. In some embodiments, the antibody or antigen binding fragment comprises a V_{H} and a V_{L} comprising the amino acid sequences set forth as SEQ ID NOs: 105 and 109, respectively, and specifically binds to hMPV F protein and neutralizes hMPV.

o. Monoclonal Antibody MPV488

In some embodiments, the antibody or antigen binding fragment is based on or derived from the MPV488 antibody, and specifically binds to hMPV F protein and neutralizes hMPV.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the MPV488 antibody, and specifically binds to hMPV F protein and neutralizes hMPV.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 113, and specifically binds to hMPV F protein and neutralizes hMPV. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 117, and specifically binds to hMPV F protein and neutralizes hMPV. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 113 and 117, respectively, and binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 114, 115, and 116, respectively, and/or a V_{L} comprising a LCDR1, a LCDR2, and a LCDR3 as set forth as SEQ ID NOs: 118, 119, and 120, respectively, and specifically binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 114, 115, and 116, respectively, a V_{L} comprising a LCDR1, a LCDR2, and a LCDR3 as set forth as SEQ ID NOs: 118, 119, and 120, respectively, wherein the V_{H} comprises an amino acid sequence at least 90% identical to SEQ ID NO: 113, such as 95%, 96%, 97%, 98% or 99% identical to SEQ ID NO: 113, and wherein the V_{L} comprises an amino acid sequence at least 90% identical to SEQ ID NO: 117, such as 95%, 96%, 97%, 98% or 99% identical to SEQ ID NO: 117, and the antibody or antigen binding fragment specifically binds to hMPV F protein and neutralizes hMPV. In this embodiment, variations due to sequence identify fall outside the CDRs.

In some embodiments, the antibody or antigen binding fragment comprises a V_{H} comprising the amino acid sequence set forth as SEQ ID NO: 113, and specifically binds to hMPV F protein and neutralizes hMPV. In more embodiments, the antibody or antigen binding fragment comprises a V_{L} comprising the amino acid sequence set forth as SEQ ID NO: 117, and specifically binds to hMPV F protein and neutralizes hMPV. In some embodiments, the antibody or antigen binding fragment comprises a V_{H} and a V_{L} comprising the amino acid sequences set forth as SEQ ID NOs: 113 and 117, respectively, and specifically binds to hMPV F protein and neutralizes hMPV.

p. Monoclonal Antibody MPV489

In some embodiments, the antibody or antigen binding fragment is based on or derived from the MPV489 antibody, and specifically binds to hMPV F protein and neutralizes hMPV.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the MPV489 antibody, and specifically binds to hMPV F protein and neutralizes hMPV.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 121, and specifically binds to hMPV F protein and neutralizes hMPV. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 125, and specifically binds to hMPV F protein and neutralizes hMPV. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 121 and 125, respectively, and specifically binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 122, 123, and 124, respectively, and/or a V_{L} comprising a LCDR1, a LCDR2, and a LCDR3 as set forth as SEQ ID NOs: 126, 127, and 128, respectively, and specifically binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 122, 123, and 124, respectively, a V_{L} comprising a LCDR1, a LCDR2, and a LCDR3 as set forth as SEQ ID NOs: 126, 127, and 128, respectively, wherein the V_{H} comprises an amino acid sequence at least 90% identical to SEQ ID NO: 121, such as 95%, 96%, 97%, 98% or 99% identical to SEQ ID NO: 121, and wherein the V_{L} comprises an amino acid sequence at least 90% identical to SEQ ID NO: 125, such as 95%, 96%, 97%, 98% or 99% identical to SEQ ID NO: 125, and the antibody or antigen binding fragment specifically binds to hMPV F protein and neutralizes hMPV. In this embodiment, variations due to sequence identify fall outside the CDRs.

In some embodiments, the antibody or antigen binding fragment comprises a V_{H} comprising the amino acid sequence set forth as SEQ ID NO: 121, and specifically binds to hMPV F protein and neutralizes hMPV. In more embodiments, the antibody or antigen binding fragment comprises a V_{L} comprising the amino acid sequence set forth as SEQ ID NO: 125, and specifically binds to hMPV F protein and neutralizes hMPV. In some embodiments, the antibody or antigen binding fragment comprises a V_{H} and a V_{L} comprising the amino acid sequences set forth as SEQ ID NOs: 121 and 125, respectively, and specifically binds to hMPV F protein and neutralizes hMPV.

q. Monoclonal Antibody MPV491

In some embodiments, the antibody or antigen binding fragment is based on or derived from the MPV491 antibody, and specifically binds to hMPV F protein and neutralizes hMPV.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to TMGT, Kabat or Chothia), of the MPV491 antibody, and specifically binds to hMPV F protein and neutralizes hMPV.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 129, and specifically binds to hMPV F protein and neutralizes hMPV. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 133, and specifically binds to hMPV F protein and neutralizes hMPV. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 129 and 133, respectively, and specifically binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 130, 131, and 132, respectively, and/or a V_{L} comprising a LCDR1, a LCDR2, and a LCDR3 as set forth as SEQ ID NOs: 134, 135, and 136, respectively, and specifically binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 130, 131, and 132, respectively, a V_{L} comprising a LCDR1, a LCDR2, and a LCDR3 as set forth as SEQ ID NOs: 134, 135, and 136, respectively, wherein the V_{H} comprises an amino acid sequence at least 90% identical to SEQ ID NO: 129, such as 95%, 96%, 97%, 98% or 99% identical to SEQ ID NO: 129, and wherein the V_{L} comprises an amino acid sequence at least 90% identical to SEQ ID NO: 133, such as 95%, 96%, 97%, 98% or 99% identical to SEQ ID NO: 133, and the antibody or antigen binding fragment specifically binds to hMPV F protein and neutralizes hMPV. In this embodiment, variations due to sequence identify fall outside the CDRs.

In some embodiments, the antibody or antigen binding fragment comprises a V_{H} comprising the amino acid sequence set forth as SEQ ID NO: 129, and specifically binds to hMPV F protein and neutralizes hMPV. In more embodiments, the antibody or antigen binding fragment comprises a V_{L} comprising the amino acid sequence set forth as SEQ ID NO: 133, and specifically binds to hMPV F protein and neutralizes hMPV. In some embodiments, the antibody or antigen binding fragment comprises a V_{H} and a V_{L} comprising the amino acid sequences set forth as SEQ ID NOs: 129 and 133, respectively, and specifically binds to hMPV F protein and neutralizes hMPV.

r. Monoclonal Antibody MPV503

In some embodiments, the antibody or antigen binding fragment is based on or derived from the MPV503 antibody, and specifically binds to hMPV F protein and neutralizes hMPV.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the MPV503 antibody, and specifically binds to hMPV F protein and neutralizes hMPV.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 137, and specifically binds to hMPV F protein and neutralizes hMPV. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 141, and specifically binds to hMPV F protein and neutralizes hMPV. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 137 and 141, respectively, and specifically binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 138, 139, and 140, respectively, and/or a V_{L} comprising a LCDR1, a LCDR2, and a LCDR3 as set forth as SEQ ID NOs: 142, 143, and 144, respectively, and specifically binds to hMPV F protein and neutralizes hMPV.

In some embodiments, the antibody or antigen binding fragment comprises a V_{H} comprising a HCDR1, a HCDR2, and a HCDR3 as set forth as SEQ ID NOs: 138, 139, and 140, respectively, a V_{L} comprising a LCDR1, a LCDR2, and a LCDR3 as set forth as SEQ ID NOs: 142, 143, and 144, respectively, wherein the V_{H} comprises an amino acid sequence at least 90% identical to SEQ ID NO: 137, such as 95%, 96%, 97%, 98% or 99% identical to SEQ ID NO: 137, and wherein the V_{L} comprises an amino acid sequence at least 90% identical to SEQ ID NO: 141, such as 95%, 96%, 97%, 98% or 99% identical to SEQ ID NO: 141, and the antibody or antigen binding fragment specifically binds to hMPV F protein and neutralizes hMPV. In this embodiment, variations due to sequence identify fall outside the CDRs.

In some embodiments, the antibody or antigen binding fragment comprises a V_{H} comprising the amino acid sequence set forth as SEQ ID NO: 137, and specifically binds to hMPV F protein and neutralizes hMPV. In more embodiments, the antibody or antigen binding fragment comprises a V_{L} comprising the amino acid sequence set forth as SEQ ID NO: 141, and specifically binds to hMPV F protein and neutralizes hMPV. In some embodiments, the antibody or antigen binding fragment comprises a V_{H} and a V_{L} comprising the amino acid sequences set forth as SEQ ID NOs: 137 and 141, respectively, and specifically binds to hMPV F protein and neutralizes hMPV.

1. Additional Antibodies that Bind to the MP467 Epitope on MPV F Protein

It is disclosed herein that MPV467 targets the hMPV F protein and the antibody binds pre-fusion F protein with a higher affinity than post-fusion F protein. The binding pose of MPV467 is disclosed, specifically that it binds a prefusion-specific epitope overlapping antigenic sites II and V on a single protomer. The helix-turn-helix (α6-α7) located within antigenic site II that is bound by MPV467 does not undergo a conformational change between pre- and post-fusion states. Accordingly, in some embodiments, an antibody or antigen binding fragment is provided that specifically binds to an epitope on hMPV F protein that is bound by MPV467, wherein the antibodies do not cross-react with the RSV F protein. In other embodiments, an antibody or antigen binding fragment is provided that specifically binds to an epitope on hMPV F protein that is bound by any of the disclosed antibodies.

In some examples, antibodies that bind to an epitope of interest can be identified based on their ability to cross-compete (for example, to competitively inhibit the binding of, in a statistically significant manner), such as with the MPV467 antibody provided herein, in binding assays. In other examples, antibodies that bind to an epitope of interest can be identified based on their ability to cross-compete (for example, to competitively inhibit the binding of, in a statistically significant manner) with the MPV467 antibody provided herein, or with any of the disclosed antibodies, in binding assays.

Human antibodies that bind to the same epitope on hMPV F protein to which the MPV467 antibody binds, or to which any of the disclosed antibodies bind, can be produced using any suitable method. Such antibodies may be prepared, for example, by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies that bind to the same epitope on hMPV F protein to which the MPV467 antibody binds, or to which any of the disclosed antibodies bind, can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3): 185-91 (2005). Human antibodies may also be generated by isolating Fv clone variable domain sequences selected from human-derived phage display libraries. Such variable domain sequences may then be combined with a desired human constant domain.

Antibodies and antigen binding fragments that specifically bind to the same epitope on hMPV F protein to which the MPV467 antibody binds, or that bind to the same epitope on hMPV F protein to which any of the disclosed antibodies bind, can also be isolated by screening combinatorial libraries for antibodies with the desired binding characteristics. For example, by generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

### 2. Additional Description of Antibodies and Antigen Binding Fragments

The antibody or antigen binding fragment can be a human antibody or fragment thereof. Chimeric antibodies are also provided. The antibody or antigen binding fragment can include any suitable framework region, such as (but not limited to) a human framework region from another source, or an optimized framework region. Alternatively, a heterologous framework region, such as, but not limited to a mouse or monkey framework region, can be included in the heavy or light chain of the antibodies.

The antibody can be of any isotype. The antibody can be, for example, an IgM or an IgG antibody, such as IgG₁, IgG₂, IgG₃, or IgG₄. The class of an antibody that specifically binds hMPV can be switched with another. In one aspect, a nucleic acid molecule encoding V_{L} or V_{H} is isolated such that it does not include any nucleic acid sequences encoding the constant region of the light or heavy chain, respectively. A nucleic acid molecule encoding V_{L} or V_{H} is then operatively linked to a nucleic acid sequence encoding a C_{L} or C_{H} from a different class of immunoglobulin molecule. This can be achieved, for example, using a vector or nucleic acid molecule that comprises a C_{L} Or C_{H} chain. For example, an antibody that specifically binds PfCSP, that was originally IgG may be class switched to an IgM. Class switching can be used to convert one IgG subclass to another, such as from IgG₁ to IgG₂, IgG₃, or IgG₄.

In some examples, the disclosed antibodies are oligomers of antibodies, such as dimers, trimers, tetramers, pentamers, hexamers, septamers, octomers and so on.

The antibody or antigen binding fragment can be derivatized or linked to another molecule (such as another peptide or protein). In general, the antibody or antigen binding fragment is derivatized such that the binding to hMPV is not affected adversely by the derivatization or labeling. For example, the antibody or antigen binding fragment can be functionally linked (by chemical coupling, genetic fusion, noncovalent association or otherwise) to one or more other molecular entities, such as another antibody (for example, a bi-specific antibody or a diabody), a detectable marker, an effector molecule, or a protein or peptide that can mediate association of the antibody or antibody portion with another molecule (such as a streptavidin core region or a polyhistidine tag).

### (a) Binding Affinity

In several embodiments, the antibody or antigen binding fragment specifically binds hMPV F protein with an affinity (e.g., measured by K_{D}) of no more than 1.0×10⁻⁸ M, no more than 5.0×10⁻⁵ M, no more than 1.0×10⁻⁹ M, no more than 5.0×10⁻⁹ M, no more than 1.0×10⁻¹⁰ M, no more than 5.0×10⁻¹⁰ M, or no more than 1.0×10⁻¹¹ M. K_{D} can be measured, for example, by a radiolabeled antigen binding assay (RIA) performed with the Fab version of an antibody of interest and its antigen. In one assay, solution binding affinity of Fabs for antigen is measured by equilibrating Fab with a minimal concentration of (¹²⁵I) labeled antigen in the presence of a titration series of unlabeled antigen, then capturing bound antigen with an anti-Fab antibody-coated plate (see, e.g., Chen et al., J. Mol. Biol. 293(4):865-881, 1999). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (NUNC™ Catalog #269620), 100 μM or 26 μM [¹²⁵I]-antigen are mixed with serial dilutions of a Fab of interest (e.g., consistent with assessment of the anti-VEGF antibody, Fab-12, in Presta et al., Cancer Res. 57(20):4593-4599, 1997). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT™-20; PerkinEmler) is added, and the plates are counted on a TOPCOUNT™ gamma counter (PerkinEmler) for ten minutes. Concentrations of each Fab that give less than or equal to 20% of maximal binding are chosen for use in competitive binding assays.

In another assay, K_{D} can be measured using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE®, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 1/min. Association rates (kₒₙ) and dissociation rates (k_{off}) are calculated using a simple one-to-one Langmuir binding model (BIACORE®Evaluation Software version 3.2) by simultaneously fitting the association and dissociation sensorgrams. The equilibrium dissociation constant (K_{D}) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### (b) Multispecific Antibodies

In some embodiments, a multi-specific antibody, such as a bi-specific antibody, is provided that comprises an antibody or antigen binding fragment that specifically binds hMPV, as provided herein, or an antigen binding fragment thereof. Any suitable method can be used to design and produce the multi-specific antibody, such as crosslinking two or more antibodies, antigen binding fragments (such as scFvs) of the same type or of different types. Exemplary methods of making multispecific antibodies include those described in PCT Pub. No. WO2013/163427, which is incorporated by reference herein in its entirety. Non-limiting examples of suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate).

The multi-specific antibody may have any suitable format that allows for binding to hMPV F protein by the antibody or antigen binding fragment as provided herein. Bispecific single chain antibodies can be encoded by a single nucleic acid molecule. Non-limiting examples of bispecific single chain antibodies, as well as methods of constructing such antibodies are provided in U.S. Pat. Nos. 8,076,459, 8,017,748, 8,007,796, 7,919,089, 7,820,166, 7,635,472, 7,575,923, 7,435,549, 7,332,168, 7,323,440, 7,235,641, 7,229,760, 7,112,324, 6,723,538. Additional examples of bispecific single chain antibodies can be found in PCT application No. WO 99/54440; Mack et al., J. Immunol., 158(8):3965-3970, 1997; Mack et al., Proc. Natl. Acad. Sci. U.S.A., 92(15):7021-7025, 1995; Kufer et al., Cancer Immunol. Immunother., 45(3-4):193-197, 1997; Löffler et al., Blood, 95(6):2098-2103, 2000; and Bruhl et al., J. Immunol., 166(4):2420-2426, 2001. Production of bispecific Fab-scFv (“bibody”) molecules are described, for example, in Schoonjans et al. (J. Immunol., 165(12):7050-7057, 2000) and Willems et al. (J. Chromatogr. B Analyt. Technol. Biomed Life Sci. 786(1-2):161-176, 2003). For bibodies, a scFv molecule can be fused to one of the VL-CL (L) or VH-CH1 chains, e.g., to produce a bibody one scFv is fused to the C-term of a Fab chain.

### (c) Antigen Binding Fragments

Antigen binding fragments are encompassed by the present disclosure, such as Fab, F(ab′)₂, and Fv which include a heavy chain and V_{L} and specifically bind hMPV F protein. These antibody fragments retain the ability to selectively bind with the antigen and are “antigen-binding” fragments. Non-limiting examples of such fragments include:
(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule can be obtained by treating whole antibody with pepsin, followed by reduction, to yield an intact light chain and a portion of the heavy chain;
(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;
(4) Fv, a genetically engineered fragment containing the V_{L} and V_{L} expressed as two chains; and
(5) Single chain antibody (such as scFv), defined as a genetically engineered molecule containing the V_{H} and the V_{L} linked by a suitable polypeptide linker as a genetically fused single chain molecule (see, e.g., Ahmad et al., Clin. Dev. Immunol., 2012, doi:10.1155/2012/980250; Marbry and Snavely, IDrugs, 13(8):543-549, 2010). The intramolecular orientation of the V_{H}-domain and the V_{L}-domain in a scFv, is not decisive for the provided antibodies (e.g., for the provided multispecific antibodies). Thus, scFvs with both possible arrangements (V_{H}-domain-linker domain-V_{L}-domain; V_{L}-domain-linker domain-V_{H}-domain) may be used.
(6) A dimer of a single chain antibody (scFV₂), defined as a dimer of a scFV. This has also been termed a “miniantibody.”

Any suitable method of producing the above-discussed antigen binding fragments may be used. Non-limiting examples are provided in Harlow and Lane, Antibodies: A Laboratory Manual, 2^{nd}, Cold Spring Harbor Laboratory, New York, 2013.

Antigen binding fragments can be prepared by proteolytic hydrolysis of the antibody or by expression in a host cell (such as an E. coli cell) of DNA encoding the fragment. Antigen binding fragments can also be obtained by pepsin or papain digestion of whole antibodies by conventional methods. For example, antigen binding fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

### (d) Variants

In some embodiments, amino acid sequence variants of the antibodies provided herein (such as MPV467 or any of the disclosed antibodies) are provided. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some embodiments, antibody variants having one or more amino acid substitutions are provided. Sites of interest for substitutional mutagenesis include the CDRs and the framework regions. Amino acid substitutions may be introduced into an antibody of interest and the products screened for a desired activity, e.g., retained/improved antigen binding, decreased immunogenicity, or improved ADCC or CDC.

The variants typically retain amino acid residues necessary for correct folding and stabilizing between the V_{H} and the V_{L} regions, and will retain the charge characteristics of the residues in order to preserve the low pI and low toxicity of the molecules. Amino acid substitutions can be made in the V_{H} and the V_{L} regions to increase yield.

In some embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 1. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 5.

In some embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 9. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 13.

In some embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 17. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 21.

In some embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 25. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 29.

In some embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 33. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 37.

In some embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 41. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 45.

In some embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 49. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 53.

In some embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 57. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 61.

In some embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 65. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 69.

In some embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 73. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 77.

In some embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 81. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 85.

In some embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 89. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 93.

In some embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 97. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 101.

In some embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 105. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 109.

In some embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 113. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 117.

In some embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 121. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 125.

In some embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 129. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 133.

In some embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 137. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 141.

In some embodiments, the antibody or antigen binding fragment can include up to 10 (such as up to 1, up to 2, up to 3, up to 4, up to 5, up to 6, up to 7, up to 8, or up to 9) amino acid substitutions (such as conservative amino acid substitutions) in the framework regions of the heavy chain of the antibody, or the light chain of the antibody, or the heavy and light chains of the antibody, compared to known framework regions, or compared to the framework regions of the MPV86, MPV414, MPV454, MPV456, MPV464, MPV467, MPV477, MPV478, MPV481, MPV482, MPV483, MPV485, MPV486, MPV487, MPV488, MPV489, MPV491, or MPV503 antibody, and maintain the specific binding activity for hMPV F protein.

In some embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some embodiments of the variant V_{H} and V_{L} sequences provided above, each CDR either is unaltered, or contains no more than one, two or three amino acid substitutions.

To increase binding affinity of the antibody, the V_{L} and V_{H} segments can be randomly mutated, such as within HCDR3 region or the LCDR3 region, in a process analogous to the in vivo somatic mutation process responsible for affinity maturation of antibodies during a natural immune response. Thus in vitro affinity maturation can be accomplished by amplifying V_{H} and V_{L} regions using PCR primers complementary to the HCDR3 or LCDR3, respectively. In this process, the primers have been “spiked” with a random mixture of the four nucleotide bases at certain positions such that the resultant PCR products encode V_{H} and V_{L} segments into which random mutations have been introduced into the V_{H} and/or V_{L} CDR3 regions. These randomly mutated V_{H} and V_{L} segments can be tested to determine the binding affinity for hMPV F protein.

In some embodiments, an antibody (such as MPV86, MPV414, MPV454, MPV456, MPV464, MPV467, MPV477, MPV478, MPV481, MPV482, MPV483, MPV485, MPV486, MPV487, MPV488, MPV489, MPV491, or MPV503) or antigen binding fragment is altered to increase or decrease the extent to which the antibody or antigen binding fragment is glycosylated. Addition or deletion of glycosylation sites may be conveniently accomplished by altering the amino acid sequence such that one or more glycosylation sites is created or removed.

Where the antibody (such as MPV86, MPV414, MPV454, MPV456, MPV464, MPV467, MPV477, MPV478, MPV481, MPV482, MPV483, MPV485, MPV486, MPV487, MPV488, MPV489, MPV491, or MPV503) comprises an Fc region, the carbohydrate attached thereto may be altered. Native antibodies produced by mammalian cells typically comprise a branched, biantennary oligosaccharide that is generally attached by an N-linkage to Asn297 of the CH₂ domain of the Fc region. See, e.g., Wright et al. Trends Biotechnol. 15(1):26-32, 1997. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region; however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO 2002/031140; Okazaki et al., J. Mol. Biol., 336(5):1239-1249, 2004; Yamane-Ohnuki et al., Biotechnol. Bioeng. 87(5):614-622, 2004. Examples of cell lines capable of producing defucosylated antibodies include Lee 13 CHO cells deficient in protein fucosylation (Ripka et al., Arch. Biochem. Biophys. 249(2):533-545, 1986; US Pat. Appl. No. US 2003/0157108 and WO 2004/056312, especially at Example 11), and knockout cell lines, such as alpha-1,6-fucosyltransferase gene, FUT8, knockout CHO cells (see, e.g., Yamane-Ohnuki et al., Biotechnol. Bioeng., 87(5): 614-622, 2004; Kanda et al., Biotechnol. Bioeng., 94(4):680-688, 2006; and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

In several embodiments, the constant region of the antibody ((such as MPV86, MPV414, MPV454, MPV456, MPV464, MPV467, MPV477, MPV478, MPV481, MPV482, MPV483, MPV485, MPV486, MPV487, MPV488, MPV489, MPV491, or MPV503) comprises one or more amino acid substitutions to optimize in vivo half-life of the antibody. The serum half-life of IgG Abs is regulated by the neonatal Fc receptor (FcRn). Thus, in several embodiments, the antibody comprises an amino acid substitution that increases binding to the FcRn. Non-limiting examples of such substitutions include substitutions at IgG constant regions T250Q and M428L (see, e.g., Hinton et al., J Immunol., 176(1):346-356, 2006); M428L and N434S (the “LS” mutation, see, e.g., Zalevsky, et al., Nature Biotechnol., 28(2):157-159, 2010); N434A (see, e.g., Petkova et al., Int. Immunol., 18(12):1759-1769, 2006); T307A, E380A, and N434A (see, e.g., Petkova et al., Int. Immunol., 18(12):1759-1769, 2006); and M252Y, S254T, and T256E (see, e.g., Dall'Acqua et al., J. Biol. Chem., 281(33):23514-23524, 2006). The disclosed antibodies and antigen binding fragments can be linked to or comprise an Fc polypeptide including any of the substitutions listed above, for example, the Fe polypeptide can include the M428L and N434S substitutions.

In some embodiments, the constant region of the antibody comprises one or more amino acid substitutions to optimize ADCC. ADCC is mediated primarily through a set of closely related Fey receptors. In some embodiments, the antibody comprises one or more amino acid substitutions that increase binding to FcγRIIIa. Non-limiting examples of such substitutions include substitutions at IgG constant regions S239D and I332E (see, e.g., Lazar et al., Proc. Natl., Acad. Sci. U.S.A., 103(11):4005-4010, 2006); and S239D, A330L, and I332E (see, e.g., Lazar et al., Proc. Natl., Acad. Sci. U.S.A., 103(11):4005-4010, 2006).

Combinations of the above substitutions are also included, to generate an IgG constant region with increased binding to FcRn and FcγRIIIa. The combinations increase antibody half-life and ADCC. For example, such combinations include antibodies with the following amino acid substitutions in the Fc region: (1) S239D/I332E and T250Q/M428L; (2) S239D/I332E and M428L/N434S; (3) S239D/I332E and N434A; (4) S239D/I332E and T307A/E380A/N434A; (5) S239D/I332E and M252Y/S254T/T256E; (6) S239D/A330L/I332E and 250Q/M428L; (7) S239D/A330L/I332E and M428L/N434S; (8) S239D/A330L/I332E and N434A; (9) S239D/A330L/I332E and T307A/E380A/N434A; or (10) S239D/A330L/I332E and M252Y/S254T/T256E. In some examples, the antibodies, or an antigen binding fragment thereof is modified such that it is directly cytotoxic to infected cells, or uses natural defenses such as complement, ADCC, or phagocytosis by macrophages.

In some embodiments, an antibody provided herein may be further modified to contain additional nonproteinaceous moieties. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in an application under defined conditions, etc.

### B. Conjugates

The antibodies and antigen binding fragments that specifically bind to hMPV F protein, as disclosed herein, can be conjugated to an agent, such as an effector molecule or detectable marker. Both covalent and noncovalent attachment means may be used. Various effector molecules and detectable markers can be used, including (but not limited to) toxins and radioactive agents such as ¹²⁵I, ³²P, ¹⁴C, ³H and ³⁵S and other labels, target moieties and ligands, etc. The choice of a particular effector molecule or detectable marker depends on the particular target molecule or cell, and the desired biological effect.

The procedure for attaching an effector molecule or detectable marker to an antibody or antigen binding fragment varies according to the chemical structure of the effector. Polypeptides typically contain a variety of functional groups, such as carboxyl (—COOH), free amine (—NH₂) or sulfhydryl (—SH) groups, which are available for reaction with a suitable functional group on a polypeptide to result in the binding of the effector molecule or detectable marker. Alternatively, the antibody or antigen binding fragment is derivatized to expose or attach additional reactive functional groups. The derivatization may involve attachment of any suitable linker molecule. The linker is capable of forming covalent bonds to both the antibody or antigen binding fragment and to the effector molecule or detectable marker. Suitable linkers include, but are not limited to, straight or branched-chain carbon linkers, heterocyclic carbon linkers, or peptide linkers. Where the antibody or antigen binding fragment and the effector molecule or detectable marker are polypeptides, the linkers may be joined to the constituent amino acids through their side chains (such as through a disulfide linkage to cysteine) or the alpha carbon, or through the amino, and/or carboxyl groups of the terminal amino acids.

In view of the large number of methods that have been reported for attaching a variety of radiodiagnostic compounds, radiotherapeutic compounds, labels (such as enzymes or fluorescent molecules), toxins, and other agents to antibodies, a suitable method for attaching a given agent to an antibody or antigen binding fragment or other polypeptide can be determined.

The antibody or antigen binding fragment can be conjugated with a detectable marker; for example, a detectable marker capable of detection by ELISA, spectrophotometry, flow cytometry, microscopy or diagnostic imaging techniques (such as CT, computed axial tomography (CAT), MRI, magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, green fluorescent protein (GFP), and yellow fluorescent protein (YFP). An antibody or antigen binding fragment can also be conjugated with enzymes that are useful for detection, such as horseradish peroxidase, β-galactosidase, luciferase, alkaline phosphatase, glucose oxidase and the like. When an antibody or antigen binding fragment is conjugated with a detectable enzyme, it can be detected by adding additional reagents that the enzyme uses to produce a reaction product that can be discerned. For example, when the agent horseradish peroxidase is present, the addition of hydrogen peroxide and diaminobenzidine leads to a colored reaction product, which is visually detectable. An antibody or antigen binding fragment may also be conjugated with biotin, and detected through indirect measurement of avidin or streptavidin binding. It should be noted that the avidin itself can be conjugated with an enzyme or a fluorescent label.

The antibody or antigen binding fragment can be conjugated with a paramagnetic agent, such as gadolinium. Paramagnetic agents such as superparamagnetic iron oxide are also of use as labels. Antibodies can also be conjugated with lanthanides (such as europium and dysprosium), and manganese. An antibody or antigen binding fragment may also be labeled with a predetermined polypeptide epitope recognized by a secondary reporter (such as leucine zipper pair sequences, binding sites for secondary antibodies, metal binding domains, epitope tags).

The antibody or antigen binding fragment can also be conjugated with a radiolabeled amino acid, for example, for diagnostic purposes. For instance, the radiolabel may be used to detect hMPV by radiography, emission spectra, or other diagnostic techniques. Examples of labels for polypeptides include, but are not limited to, the following radioisotopes: ³H, ¹⁴C, ³⁵S, ⁹⁰Y, ^{99m}Tc, ¹¹¹In, ¹²⁵I, ¹³¹I. The radiolabels may be detected, for example, using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The average number of effector molecule or detectable marker moieties per antibody or antigen binding fragment in a conjugate can range, for example, from 1 to 20 moieties per antibody or antigen binding fragment. In some embodiments, the average number of effector molecules or detectable marker moieties per antibody or antigen binding fragment in a conjugate range from about 1 to about 2, from about 1 to about 3, about 1 to about 8; from about 2 to about 6; from about 3 to about 5; or from about 3 to about 4. The loading (for example, effector molecule per antibody ratio) of a conjugate may be controlled in different ways, for example, by: (i) limiting the molar excess of effector molecule-linker intermediate or linker reagent relative to antibody, (ii) limiting the conjugation reaction time or temperature, (iii) partial or limiting reducing conditions for cysteine thiol modification, (iv) engineering by recombinant techniques the amino acid sequence of the antibody such that the number and position of cysteine residues is modified for control of the number or position of linker-effector molecule attachments.

### C. Polynucleotides and Expression

Nucleic acid molecules (for example, cDNA or RNA molecules) encoding the amino acid sequences of antibodies, antigen binding fragments, and conjugates that specifically bind to hMPV, as disclosed herein, are provided. Nucleic acids encoding these molecules can readily be produced using the amino acid sequences provided herein (such as the CDR sequences and V_{H} and V_{L} sequences), sequences available in the art (such as framework or constant region sequences), and the genetic code. In several embodiments, nucleic acid molecules can encode the V_{H}, the V_{L}, or both the V_{H} and V_{L} (for example in a bicistronic expression vector) of a disclosed antibody or antigen binding fragment. In some embodiments, the nucleic acid molecules encode an scFv. In several embodiments, the nucleic acid molecules can be expressed in a host cell (such as a mammalian cell) to produce a disclosed antibody or antigen binding fragment.

The genetic code can be used to construct a variety of functionally equivalent nucleic acid sequences, such as nucleic acids which differ in their sequence but which encode the same antibody sequence, or encode a conjugate or fusion protein including the V_{L}, and/or V_{H} nucleic acid sequence.

Nucleic acid molecules encoding the antibodies, antigen binding fragments, and conjugates that specifically bind to hMPV F protein can be prepared by any suitable method including, for example, cloning of appropriate sequences or by direct chemical synthesis by standard methods. Chemical synthesis produces a single stranded oligonucleotide. This can be converted into double stranded DNA by hybridization with a complementary sequence or by polymerization with a DNA polymerase using the single strand as a template.

Exemplary nucleic acids can be prepared by cloning techniques. Examples of appropriate cloning and sequencing techniques can be found, for example, in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements).

Nucleic acids can also be prepared by amplification methods. Amplification methods include the polymerase chain reaction (PCR), the ligase chain reaction (LCR), the transcription-based amplification system (TAS), and the self-sustained sequence replication system (3SR).

The nucleic acid molecules can be expressed in a recombinantly engineered cell such as bacteria, plant, yeast, insect and mammalian cells. The antibodies, antigen binding fragments, and conjugates can be expressed as individual proteins including the V_{H} and/or V_{L} (linked to an effector molecule or detectable marker as needed), or can be expressed as a fusion protein. Any suitable method of expressing and purifying antibodies and antigen binding fragments may be used; non-limiting examples are provided in Al-Rubeai (Ed.), Antibody Expression and Production, Dordrecht; New York: Springer, 2011). An immunoadhesin can also be expressed. Thus, in some examples, nucleic acids encoding a V_{H} and V_{L}, and immunoadhesin are provided. The nucleic acid sequences can optionally encode a leader sequence.

To create a scFv the V_{H}- and V_{L}-encoding DNA fragments can be operatively linked to another fragment encoding a flexible linker, e.g., encoding the amino acid sequence (Gly₄-Ser)₃, such that the V_{H} and V_{L} sequences can be expressed as a contiguous single-chain protein, with the V_{L} and V_{H} domains joined by the flexible linker (see, e.g., Bird et al., Science, 242(4877):423-426, 1988; Huston et al., Proc. Nat. Acad. Sci. U.S.A., 85(16):5879-5883, 1988; McCafferty et al., Nature, 348:552-554, 1990; Kontermiann and Dlbel (Eds.), Antibody Engineering, Vols. 1-2, 2^{nd} ed., Springer-Verlag, 2010; Greenfield (Ed.), Antibodies: A Laboratory Manual, 2^{nd} ed. New York: Cold Spring Harbor Laboratory Press, 2014). Optionally, a cleavage site can be included in a linker, such as a furin cleavage site.

The single chain antibody may be monovalent, if only a single V_{H} and V_{L} are used, bivalent, if two V_{H} and V_{L} are used, or polyvalent, if more than two V_{H} and V_{L} are used. Bispecific or polyvalent antibodies may be generated that bind specifically to hMPV F protein and another antigen. The encoded V_{H} and V_{L} optionally can include a furin cleavage site between the V_{H} and V_{L} domains.

One or more DNA sequences encoding the antibodies, antigen binding fragments, or conjugates can be expressed in vitro by DNA transfer into a suitable host cell. The cell may be prokaryotic or eukaryotic. Numerous expression systems available for expression of proteins including E. coli, other bacterial hosts, yeast, and various higher eukaryotic cells such as the COS, CHO, HeLa and myeloma cell lines, can be used to express the disclosed antibodies and antigen binding fragments. Methods of stable transfer, meaning that the foreign DNA is continuously maintained in the host may be used. Hybridomas expressing the antibodies of interest are also encompassed by this disclosure.

The expression of nucleic acids encoding the antibodies and antigen binding fragments described herein can be achieved by operably linking the DNA or cDNA to a promoter (which is either constitutive or inducible), followed by incorporation into an expression cassette. The promoter can be any promoter of interest, including a cytomegalovirus promoter. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signals for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned gene, it is desirable to construct expression cassettes which contain, for example, a strong promoter to direct transcription, a ribosome binding site for translational initiation (e.g., internal ribosomal binding sequences), and a transcription/translation terminator. For E. coli, this can include a promoter such as the T7, trp, lac, or lamda promoters, a ribosome binding site, and preferably a transcription termination signal. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by any suitable method such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

Modifications can be made to a nucleic acid encoding a polypeptide described herein without diminishing its biological activity. Some modifications can be made to facilitate the cloning, expression, or incorporation of the targeting molecule into a fusion protein. Such modifications include, for example, termination codons, sequences to create conveniently located restriction sites, and sequences to add a methionine at the amino terminus to provide an initiation site, or additional amino acids (such as poly His) to aid in purification steps.

Once expressed, the antibodies, antigen binding fragments, and conjugates can be purified according to standard procedures in the art, including ammonium sulfate precipitation, affinity columns, column chromatography, and the like (see, generally, Simpson et al. (Eds.), Basic methods in Protein Purification and Analysis: A Laboratory Manual. New York: Cold Spring Harbor Laboratory Press, 2009). The antibodies, antigen binding fragment, and conjugates need not be 100% pure. Once purified, partially or to homogeneity as desired, if to be used prophylatically, the polypeptides should be substantially free of endotoxin.

Methods for expression of antibodies, antigen binding fragments, and conjugates, and/or refolding to an appropriate active form, from mammalian cells, and bacteria such as E. coli have been described and are applicable to the antibodies disclosed herein. See, e.g., Greenfield (Ed.), Antibodies: A Laboratory Manual, 2^{nd} ed. New York: Cold Spring Harbor Laboratory Press, 2014, Simpson et al. (Eds.), Basic methods in Protein Purification and Analysis: A Laboratory Manual, New York: Cold Spring Harbor Laboratory Press, 2009, and Ward et al., Nature 341(6242):544-546, 1989.

### D. Methods and Compositions

1. Inhibiting an hMPV Infection

Methods are disclosed herein for the inhibition of an hMPV infection in a subject. The methods include administering to the subject an effective amount (that is, an amount effective to inhibit the hMPV infection in the subject) of a disclosed antibody, antigen binding fragment, conjugate, or a nucleic acid encoding such an antibody, antigen binding fragment, or conjugate, to a subject at risk of an hMPV infection or having an hMPV infection. The methods can be used pre-exposure or post-exposure.

The hMPV infection does not need to be completely eliminated or inhibited for the method to be effective. For example, the method can decrease the hMPV infection by a desired amount, for example by at least 10%, at least 20%, at least 50%, at least 60%, at least 70%, at least 80%, at least 90%, at least 95%, at least 98%, or even at least 100% (elimination or prevention of detectable hMPV infection) as compared to the hMPV infection in the absence of the treatment. In some embodiments, the subject can also be treated with an effective amount of an additional agent, such as an anti-viral agent.

In some embodiments, administration of an effective amount of a disclosed antibody, antigen binding fragment, conjugate, or nucleic acid molecule, inhibits the establishment of an hMPV infection and/or subsequent disease progression in a subject, which can encompass any statistically significant reduction in hMPV activity (for example, growth or invasion) or symptoms of the hMPV infection in the subject. The antibody, antigen binding fragment, conjugate, or nucleic acid molecule, can be administered by any route of administration, including systemic or local administration. In one embodiment, the administration is intranasal administration. In another embodiment, the administration is into the lung, such as by inhalation. In a further embodiment, the administration is intra-muscular.

Methods are disclosed herein for the inhibition of an hMPV replication in a subject. The methods include administering to the subject an effective amount (that is, an amount effective to inhibit hMPV replication in the subject) of a disclosed antibody, antigen binding fragment, conjugate, or a nucleic acid encoding such an antibody, antigen binding fragment, or conjugate, to a subject at risk of an hMPV infection or having an hMPV infection. The methods can be used pre-exposure or post-exposure.

Methods are disclosed for treating an hMPV infection in a subject. Methods are also disclosed for preventing an hMPV infection in a subject. These methods include administering one or more hMPV F protein-specific antibodies, antigen binding fragments, bispecific antibodies, conjugates, or nucleic acid molecule encoding such molecules, or a composition including such molecules, as disclosed herein.

Antibodies and antigen binding fragments thereof can be administered by systemically, such as by intravenous infusion or intramuscular administration. Antibodies and antigen binding fragments thereof can be administered intranasally, intramuscularly, or into the lung, such as by inhalation. Doses of the antibody or antigen binding fragment vary, but generally range between about 0.5 mg/kg to about 50 mg/kg, such as a dose of about 1 mg/kg, about 5 mg/kg, about 10 mg/kg, about 20 mg/kg, about 30 mg/kg, about 40 mg/kg, or about 50 mg/kg. In some embodiments, the dose of the antibody or antigen binding fragment can be from about 0.5 mg/kg to about 5 mg/kg, such as a dose of about 1 mg/kg, about 2 mg/kg, about 3 mg/kg, about 4 mg/kg or about 5 mg/kg. The antibody or antigen binding fragment is administered according to a dosing schedule determined by a medical practitioner. In some examples, the antibody or antigen binding fragment is administered weekly, every two weeks, every three weeks or every four weeks.

In some embodiments, the method of inhibiting the hMPV infection in a subject further comprises administration of one or more additional agents to the subject. Additional agents of interest include, but are not limited to, anti-viral agents.

In some embodiments, the method comprises administration of a first antibody that specifically binds to hMPV F protein as disclosed herein and a second antibody that also specifically binds to hMPV F protein, such as a different epitope of hMPV F protein In some embodiments, the first antibody is one of MPV86, MPV414, MPV454, MPV456, MPV464, MPV467, MPV477, MPV478, MPV481. MPV482, MPV483, MPV485, MPV486, MPV487, MPV488, MPV489, MPV491, or MPV503. In further embodiments, the second antibody is DDS7 or MPE8. In more embodiments, the first antibody is one of MPV86, MPV414, MPV454, MPV456, MPV464, MPV467, MPV477, MPV478, MPV481, MPV482, MPV483, MPV485, MPV486, MPV487, MPV488, MPV489, MPV491, or MPV503 and the second antibody is another of MPV86, MPV414, MPV454, MPV456, MPV464, MPV467, MPV477, MPV478, MPV481, MPV482, MPV483, MPV485, MPV486, MPV487, MPV488, MPV489, MPV491, or MPV503. An effective amount of one, two, three or four, five, or six of MPV86, MPV414, MPV454, MPV456, MPV464, MPV467, MPV477, MPV478, MPV481, MPV482, MPV483, MPV485, MPV486, MPV487, MPV488, MPV489, MPV491, or MPV503 can be administered to a subject. In one non-limiting example, the method includes administering an effective amount of MPV467 to the subject. The method can include administering an effective amount of one or more additional antibodies.

In some embodiments, a subject is administered DNA or RNA encoding a disclosed antibody to provide in vivo antibody production, for example using the cellular machinery of the subject. Any suitable method of nucleic acid administration may be used; non-limiting examples are provided in U.S. Pat. Nos. 5,643,578, 5,593,972 and 5,817,637. U.S. Pat. No. 5,880,103 describes several methods of delivery of nucleic acids encoding proteins to an organism. One approach to administration of nucleic acids is direct administration with plasmid DNA, such as with a mammalian expression plasmid. The nucleotide sequence encoding the disclosed antibody, or antigen binding fragments thereof, can be placed under the control of a promoter to increase expression. The methods include liposomal delivery of the nucleic acids. Such methods can be applied to the production of an antibody, or antigen binding fragments thereof. In some embodiments, a disclosed antibody or antigen binding fragment is expressed in a subject using the pVRC8400 vector (described in Barouch et al., J. Virol., 79(14), 8828-8834, 2005, which is incorporated by reference herein).

In several embodiments, a subject (such as a human subject at risk of an hMPV infection or having an hMPV infection) can be administered an effective amount of an AAV viral vector that comprises one or more nucleic acid molecules encoding a disclosed antibody or antigen binding fragment. The AAV viral vector is designed for expression of the nucleic acid molecules encoding a disclosed antibody or antigen binding fragment, and administration of the effective amount of the AAV viral vector to the subject leads to expression of an effective amount of the antibody or antigen binding fragment in the subject. Non-limiting examples of AAV viral vectors that can be used to express a disclosed antibody or antigen binding fragment in a subject include those provided in Johnson et al., Nat. Med., 15(8):901-906, 2009 and Gardner et al., Nature, 519(7541):87-91, 2015, each of which is incorporated by reference herein in its entirety.

In one embodiment, a nucleic acid encoding a disclosed antibody, or antigen binding fragment thereof, is introduced directly into tissue. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter.

Typically, the DNA is injected into muscle, although it can also be injected directly into other sites. Dosages for injection are usually around 0.5 g/kg to about 50 mg/kg, and typically are about 0.005 mg/kg to about 5 mg/kg (see, e.g., U.S. Pat. No. 5,589,466).

Single or multiple administrations of a composition including a disclosed hMPV F protein-specific antibody, antigen binding fragment, conjugate, or nucleic acid molecule encoding such molecules, can be administered depending on the dosage and frequency as required and tolerated by the patient. The dosage can be administered once, but may be administered periodically until either a desired result is achieved or until side effects warrant discontinuation of therapy. Generally, the dose is sufficient to inhibit an hMPV infection without producing unacceptable toxicity to the patient.

Data obtained from cell culture assays and animal studies can be used to formulate a range of dosage for use in humans. The dosage normally lies within a range of circulating concentrations that include the ED₅₀, with little or minimal toxicity. The dosage can vary within this range depending upon the dosage form employed and the route of administration utilized. The effective dose can be determined from cell culture assays and animal studies.

The hMPV F protein-specific antibody, antigen binding fragment, bispecific antibody, conjugate, or nucleic acid molecule encoding such molecules, or a composition including such molecules, can be administered to subjects in various ways, including local and systemic administration, such as, e.g., by injection subcutaneously, intravenously, intra-arterially, intraperitoneally, intramuscularly, intradermally, or intrathecally. In an embodiment, the antibody, antigen binding fragment, bispecific antibody, conjugate, or nucleic acid molecule encoding such molecules, or a composition including such molecules, is administered by a single subcutaneous, intravenous, intra-arterial, intraperitoneal, intramuscular, intradermal or intrathecal injection once a day. The antibody, antigen binding fragment, bispecific antibody, conjugate, or nucleic acid molecule encoding such molecules, or a composition including such molecules, can also be administered by direct injection at or near the site of disease. A further method of administration is by osmotic pump (e.g., an Alzet pump) or mini-pump (e.g., an Alzet mini-osmotic pump), which allows for controlled, continuous and/or slow-release delivery of the antibody, antigen binding fragment, conjugate, or nucleic acid molecule encoding such molecules, or a composition including such molecules, over a pre-determined period. The osmotic pump or mini-pump can be implanted subcutaneously, or near a target site.

### 2. Compositions

Compositions are provided that include one or more of the hMPV F protein-specific antibody, antigen binding fragment, conjugate, or nucleic acid molecule encoding such molecules, that are disclosed herein in a pharmaceutically acceptable carrier. In some embodiments, the composition comprises the MPV86, MPV414, MPV454, MPV456, MPV464, MPV467, MPV477, MPV478, MPV481, MPV482, MPV483, MPV485, MPV486, MPV487, MPV488, MPV489, MPV491, or MPV503 antibody disclosed herein, or an antigen binding fragment thereof. In some embodiments, the composition comprises two, three, four or more antibodies that specifically bind the hMPV F protein. In a specific non-limiting example, the antibody is MVP467. The compositions are useful, for example, for example, for the inhibition or detection of an hMPV infection. The compositions can be prepared in unit dosage forms for administration to a subject. The amount and timing of administration are at the discretion of the administering physician to achieve the desired purposes. The antibody, antigen binding fragment, conjugate, or nucleic acid molecule encoding such molecules can be formulated for systemic or local administration. In one example, the, antigen binding fragment, conjugate, or nucleic acid molecule encoding such molecules, is formulated for parenteral administration, such as intravenous administration.

In some embodiments, the antibody, antigen binding fragment, or conjugate thereof, in the composition is at least 70% (such as at least 75%, at least 80%, at least 85%, at least 90%, at least 95%, at least 96%, at least 97%, at least 98% or at least 99%) pure. In some embodiments, the composition contains less than 10% (such as less than 5%, less than 4%, less than 3%, less than 2%, less than 1%, less than 0.5%, or even less) of macromolecular contaminants, such as other mammalian (e.g., human) proteins.

The compositions for administration can include a solution of the antibody, antigen binding fragment, conjugate, or nucleic acid molecule encoding such molecules, dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by any suitable technique. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

A typical composition for intravenous administration comprises about 0.01 to about 30 mg/kg of antibody or antigen binding fragment or conjugate per subject per day (or the corresponding dose of a conjugate including the antibody or antigen binding fragment). Any suitable method may be used for preparing administrable compositions; non-limiting examples are provided in such publications as Remington: The Science and Practice of Pharmacy, 22^{nd} ed., London, UK: Pharmaceutical Press, 2013. In some embodiments, the composition can be a liquid formulation including one or more antibodies, antigen binding fragments (such as an antibody or antigen binding fragment that specifically binds to PfCSP), in a concentration range from about 0.1 mg/ml to about 20 mg/ml, or from about 0.5 mg/ml to about 20 mg/ml, or from about 1 mg/ml to about 20 mg/ml, or from about 0.1 mg/ml to about 10 mg/ml, or from about 0.5 mg/ml to about 10 mg/ml, or from about 1 mg/ml to about 10 mg/ml.

Antibodies, or an antigen binding fragment thereof or a conjugate or a nucleic acid encoding such molecules, can be provided in lyophilized form and rehydrated with sterile water before administration, although they are also provided in sterile solutions of known concentration. The antibody solution, or an antigen binding fragment or a nucleic acid encoding such antibodies or antigen binding fragments, can then be added to an infusion bag containing 0.9% sodium chloride, USP, and typically administered at a dosage of from 0.5 to 15 mg/kg of body weight. Considerable experience is available in the art in the administration of antibody drugs, which have been marketed in the U.S. since the approval of Rituximab in 1997. Antibodies, antigen binding fragments, conjugates, or a nucleic acid encoding such molecules, can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30-minute period if the previous dose was well tolerated.

Controlled-release parenteral formulations can be made as implants, oily injections, or as particulate systems. For a broad overview of protein delivery systems see, Banga, Therapeutic Peptides and Proteins: Formulation, Processing, and Delivery Systems, Lancaster, PA: Technomic Publishing Company, Inc., 1995. Particulate systems include microspheres, microparticles, microcapsules, nanocapsules, nanospheres, and nanoparticles. Microcapsules contain the active protein agent, such as a cytotoxin or a drug, as a central core. In microspheres, the active protein agent is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Krëuter, Colloidal Drug Delivery Systems, J. Kreuter (Ed.), New York, NY: Marcel Dekker, Inc., pp. 219-342, 1994; and Tice and Tabibi, Treatise on Controlled Drug Delivery: Fundamentals, Optimization, Applications, A. Kydonieus (Ed.), New York, NY: Marcel Dekker, Inc., pp. 315-339, 1992.

Polymers can be used for ion-controlled release of the antibody compositions disclosed herein. Any suitable polymer may be used, such as a degradable or nondegradable polymeric matrix designed for use in controlled drug delivery. Alternatively, hydroxyapatite has been used as a microcarrier for controlled release of proteins. In yet another aspect, liposomes are used for controlled release as well as drug targeting of the lipid-capsulated drug.

### 2. Methods of Detection and Diagnosis

Methods are also provided for the detection of the presence of hMPV F protein in vitro or in vivo. In one example, the presence of hMPV F protein is detected in a biological sample from a subject and can be used to identify a subject with an hMPV infection. The sample can be any sample, including, but not limited to, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine. The method of detection can include contacting a cell or sample, with an antibody or antigen binding fragment that specifically binds to hMPV F protein, or conjugate thereof (e.g., a conjugate including a detectable marker) under conditions sufficient to form an immune complex, and detecting the immune complex (e.g., by detecting a detectable marker conjugated to the antibody or antigen binding fragment.

In one embodiment, the antibody or antigen binding fragment is directly labeled with a detectable marker. In another embodiment, the antibody that binds the hMPV F protein (the primary antibody) is unlabeled and a secondary antibody or other molecule that can bind the primary antibody is utilized for detection. The secondary antibody is chosen that is able to specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially. Suitable labels for the antibody, antigen binding fragment or secondary antibody are known and described above, and include various enzymes, prosthetic groups, fluorescent materials, luminescent materials, magnetic agents and radioactive materials.

In some embodiments, the disclosed antibodies or antigen binding fragments thereof are used to test vaccines. For example, to test if a vaccine composition including hMPV F protein or fragment thereof assumes a prefusion conformation including the epitope of a disclosed antibody. Thus, provided herein is a method for testing a vaccine, wherein the method comprises contacting a sample containing the vaccine, such as a hMPV F protein immunogen, with a disclosed antibody or antigen binding fragment under conditions sufficient for formation of an immune complex, and detecting the immune complex, to detect the vaccine including the epitope of interest in the sample. In one example, the detection of the immune complex in the sample indicates that vaccine component, such as an hMPV F protein immunogen assumes a conformation capable of binding the antibody or antigen binding fragment.

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

### EXAMPLES

Human metapneumovirus (hMPV) is a leading cause of morbidity and hospitalization among children worldwide, however, no vaccines or therapeutics are currently available for hMPV disease prevention and treatment. The hMPV fusion (F) protein is the sole target of neutralizing antibodies. To map the immunodominant epitopes on the hMPV F protein,18 human monoclonal antibodies (mAbs) were isolated, and the mAbs were assessed for binding avidity, neutralization potency, and epitope specificity. The majority of the mAbs target diverse epitopes on the hMPV F protein, and multiple mAb binding approaches were discovered for antigenic site III. The most potent mAb, MPV467, was examined in prophylactic and therapeutic mouse challenge studies, and MPV467 limited virus replication in mouse lungs when administered 24 hrs before or 72 hrs after viral infection. In addition, this antibody was shown to be effective in a cotton rat (Sigmodon hispidus) model. The structure of MPV467 in complex with the hMPV F protein was determined using cryo-electron microscopy to a resolution of 3.3 Å, which revealed a complex prefusion-specific epitope overlapping antigenic sites II and V on a single protomer. The data revealed new insights into the immunodominant antigenic epitopes on the hMPV F protein, identified a new mAb therapy for hMPV F disease prevention and treatment, and provided the discovery of a unique pre-fusion-specific epitope on the hMPV F protein.

### Example 1

### Human Monoclonal Antibody Sequence Determinants

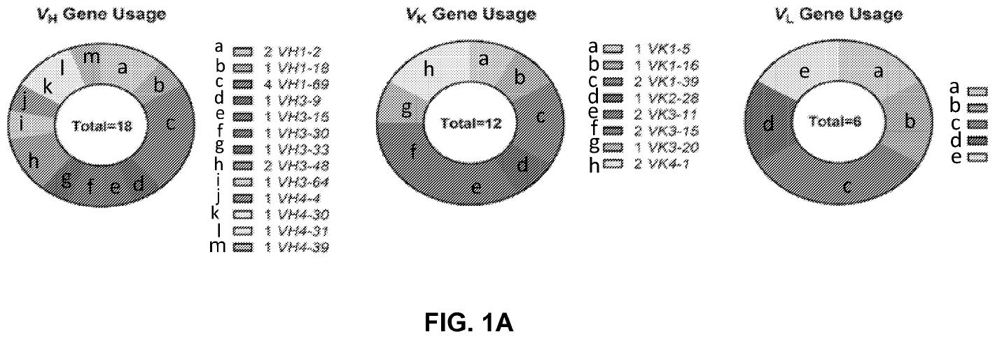

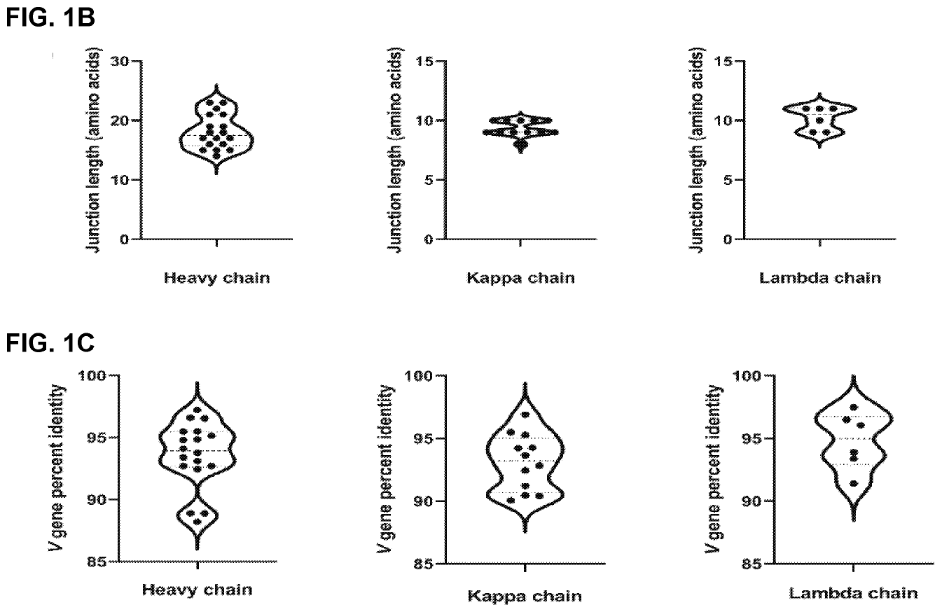

To further define the antigenic epitopes on the hMPV F protein, 18 new human mAbs were isolated using a hMPV B2 F protein (Biacchesi et al., J. Virol. 78, 12877-12887 (2004)), with sixteen mAbs generated via human hybridona technology while two were derived from antigen-specific single B cell sorting (MPV491, 503). The antibody-encoding genes were sequenced, and the results indicated the usage of a diverse set of immunoglobulin V genes across the entire panel (FIG. 1A). mAbs utilizing the VH1-69 gene were the most abundant. VH3 and VH4 gene families comprised the majority of the additional mAbs. Diversity was also present in the light chain, with eight and five unique genes utilized for the kappa and lambda mAbs, respectively. Kappa isotype mAbs utilized VK1, VK2, VK3, and VK4 gene families, while lambda isotype mAbs used VL1 and VL3 gene families. The lengths of the heavy and light chain junctions ranged from 14 to 23 amino acids for the heavy chain, 8 to 10 amino acids for the kappa chain, and 9 to 11 amino acids for the lambda chain (FIG. 1B). The percent identities of the variable genes to the germline sequence ranged 88-97% for the heavy chain (93.6% average) and 90-97% for the light chain (93.7% average) (FIG. 1C).

### Example 2

### mAb Binding and Functional Properties

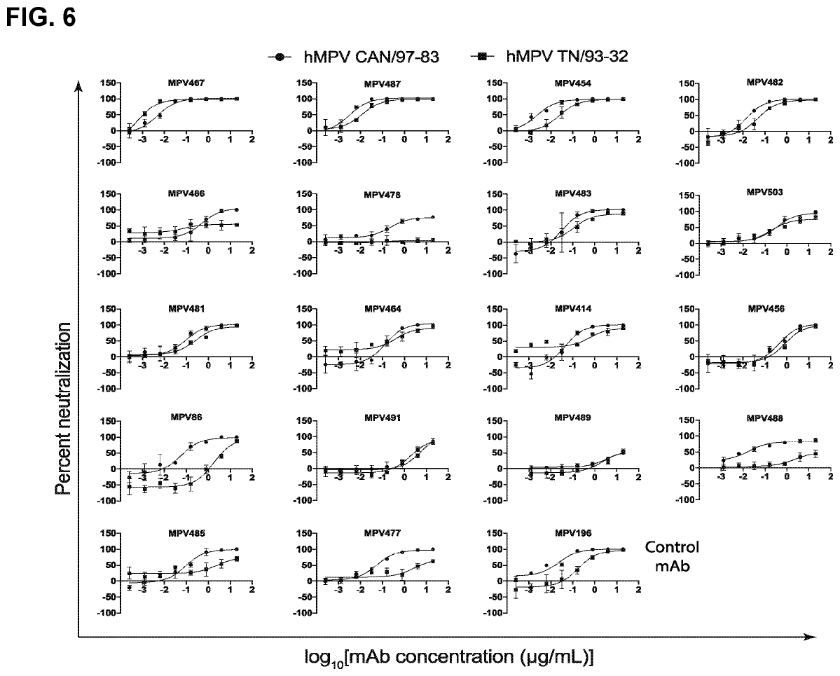

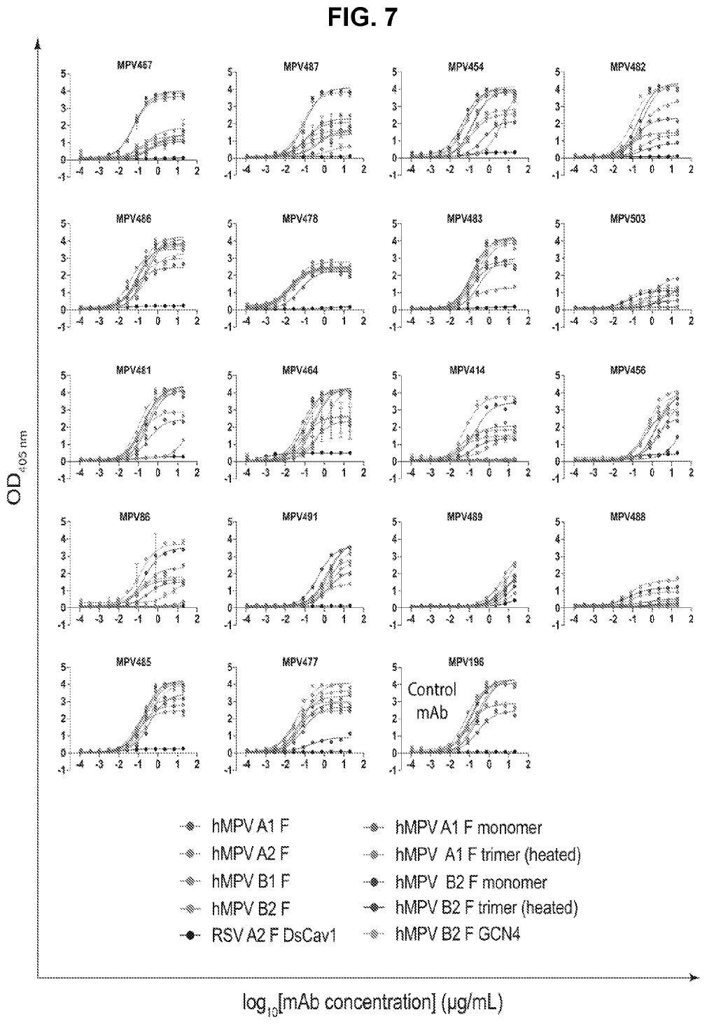

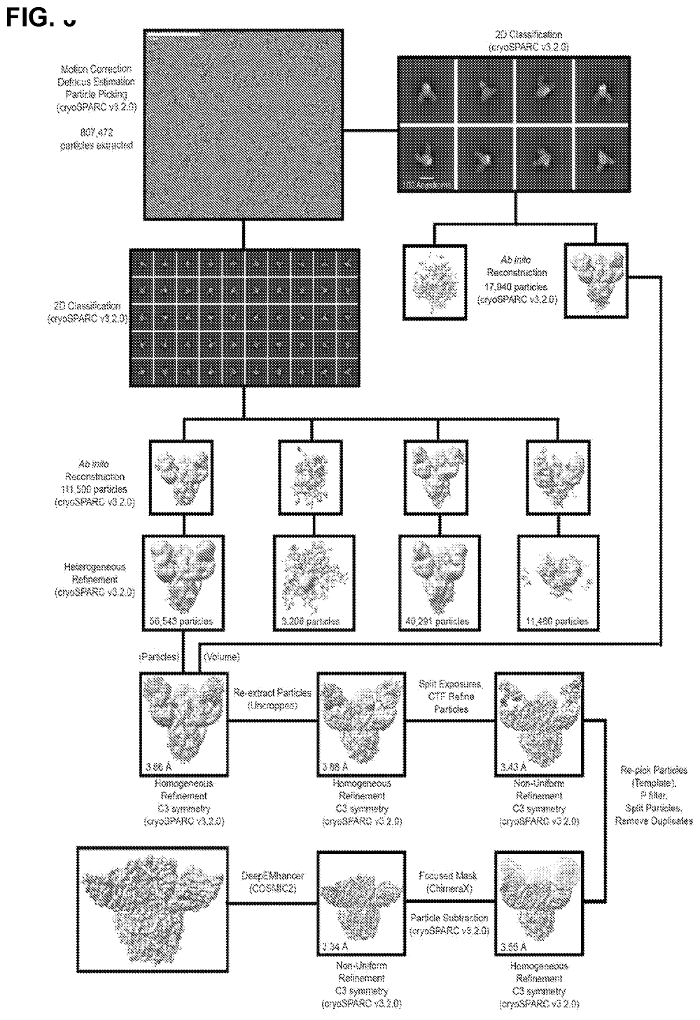

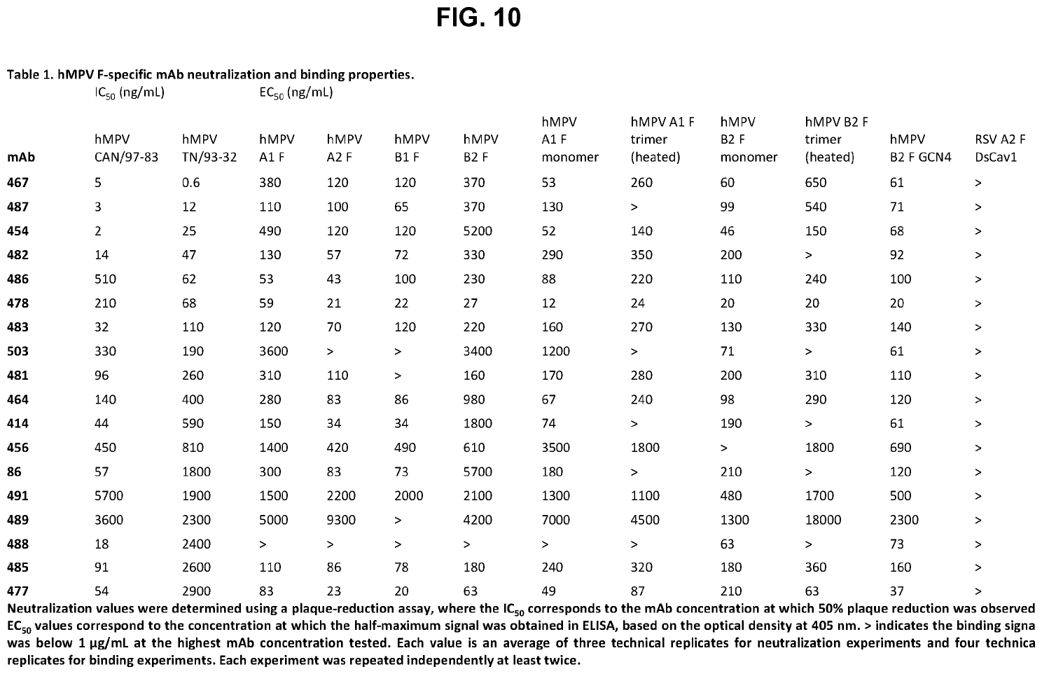

The neutralizing activity of each mAb was determined by plaque-reduction assay using representative viruses from each genotype of hMPV, i.e., hMPV CAN/97-83 (genotype A) and hMPV TN/93-32 (genotype B) (FIG. 6, FIG. 10). All mAbs had neutralizing activity against viruses from both genotypes, with mAbs MPV467, MPV487. MPV454, MPV482, and MPV488 having neutralizing activity below 20 ng/mL against hMPV CAN/97-83. mAbs MPV467, MPV487, and MPV454 have the most potent neutralizing activity against both hMPV CAN/97-83 and hMPV TN/93-32, with MPV467 reaching picomolar activity (below 1 ng/mL) against hMPV TN/93-32. mAbs MPV86, MPV488, MPV485, and MPV477 demonstrated preferential neutralization of hMPV CAN/97-83 based on at least a 20-fold lower IC₅₀ compared to hMPV TN/93-32. The binding properties of the mAbs were assessed using a panel of hMPV F proteins from each subgroup (hMPV A1 F, hMPV A2 F, hMPV B1 F, hMPV B2 F) containing mixtures of pre-fusion and post-fusion hMPV F. mAb binding to additional constructs containing exclusively monomeric pre-fusion hMPV F, post-fusion hMPV F, and a predominantly trimeric pre-fusion hMPV F (hMPV B2 F GCN4) was also assessed. Several binding patterns were observed. MPV487, MPV482, MPV503, MPV414, MPV86, and MPV488 had limited binding to post-fusion F constructs and favored binding to pre-fusion constructs. mAbs MPV467, MPV454, MPV477, MPV486, and MPV464 bound to both pre-fusion and post-fusion constructs, but had higher binding affinity for pre-fusion proteins. mAbs MPV478, MPV483, MPV481, MPV456, MPV491, MPV489, and MPV485 bound equally to both pre-fusion and post-fusion constructs.

### Example 3

### Epitope Mapping

To determine the general binding epitopes for the panel of 18 mAbs, an epitope binning experiment was conducted using biolayer interferometry as previously described (Huang and Mousa, PLOS Pathog. 16, e1008942 (2020); Bar-Peled et al., J. Virol. 93, e00342-19 (2019)). Biosensors were loaded with hMPV B2 pre-fusion F protein, associated with a test mAb, and then exposed to a control mAb with a known epitope to determine competition profiles (FIG. 2A). Control mAbs targeting known hMPV epitopes included mAbs MPE8 and MPV364 (site III), DS7 and MPV196 (DS7 epitope). 101F (site IV), and MPV458 (66-87 intratrimer epitope) (FIG. 2B). MPV481 and MPV483 were mapped to antigenic site IV, while MPV454 competed with both 101F and DS7, suggesting it binds an intermediate epitope between site IV and the DS7 site (FIG. 2B). However, MPV454 did not compete with the previously identified MPV196, which competes with DS7. MPV464, MPV491, MPV485, and MPV477 competed with both MPE8 and DS7 similar to our previous results with MPV196, MPV201, and MPV314 (Bar-Peled et al., J. Virol. 93, e00342-19 (2019)). mAbs MPV86, MPV414, MPV482, MPV487, and MPV503 were “MPV364-like,” competing with MPE8 and MPV364 but not with DS7. This differential binding mode at antigenic site III has been previously defined by competition or the lack thereof with DS7 (Bar-Peled et al., J. Virol. 93, e00342-19 (2019)). No mAbs were observed to compete with the intratrimer-targeting MPV458, although intermediate competition was observed between several mAbs and MPV458, suggesting that these may partially block binding of MPV458 or limit exposure of the intratrimer epitope bound by MPV458 centered at amino acids 66-87. MPV488 and MPV489 had partial competition with MPE8 but not MPV364, while MPV467 and MPV456 had partial competition with MPV364 but not MPE8, suggesting additional epitopes are present near antigenic site III. MPV486 and MPV478 showed partial competition with nearly all mAbs and their epitope could not be defined.

### Example 4

hMPV F-Specific mAbs Enhance Antibody-Dependent Phagocytosis of THP-1 Cells

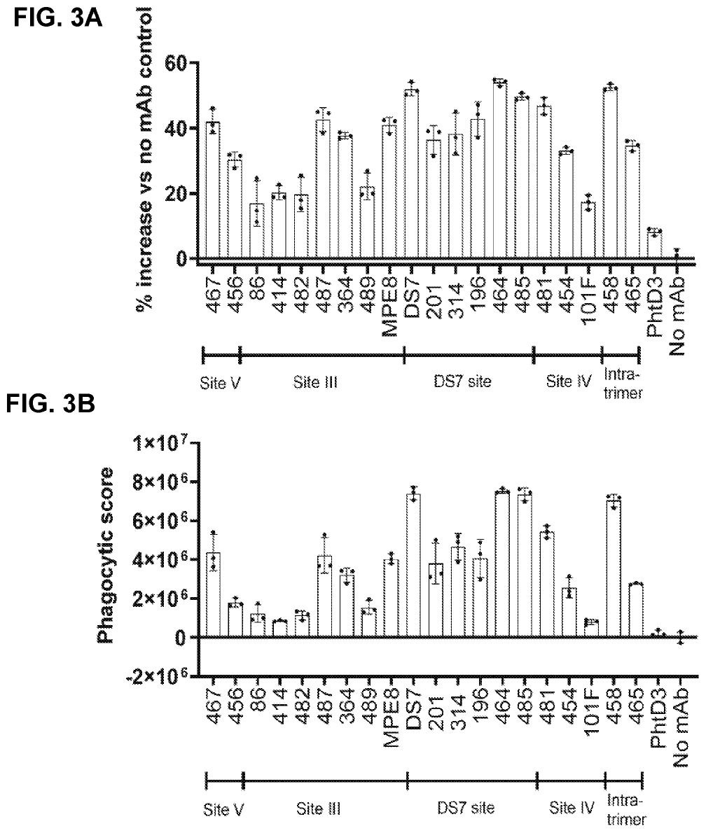

Antibodies that bind to different hMPV F antigenic sites were selected to evaluate antibody-dependent phagocytic activities (FIG. 3). All the mAbs tested significantly enhanced the phagocytosis of THP-1 cells compared to the blank and isotype control mAb (PhtD3, a mAb that binds to Streptococcus pneumoniae) controls in vitro. Antibodies that bind to the DS7-site showed overall higher phagocytic scores while the rest of the mAbs varied in ADP activity. In addition, no correlation was observed between the EC₅₀/IC₅₀ and the ADP activity of the mAbs, suggesting the ADP activities of hMPV F-specific mAbs are independent from the Fab binding epitopes. These data suggest additional protective mechanisms of anti-hMPV F mAbs beyond neutralization.

### Example 5

### Therapeutic Efficacy of MPV467

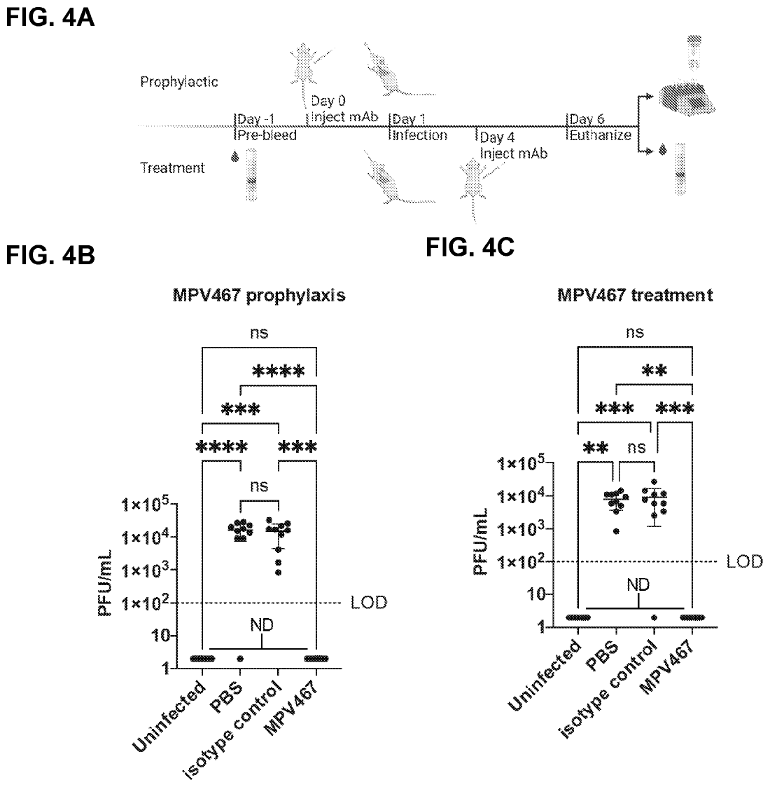

MPV467 was the most potent mAb of the panel, reaching picomolar neutralization potency against hMPV TN/93-32 and potently neutralizing hMPV CAN/97-83. This mAb has pre-fusion preference properties, as substantial binding is lost to post-fusion hMPV F proteins (FIG. 10). Based on these data, the protective efficacy of mAb MPV467 was tested in an hMPV infection model in BALB/c mice. Male and female mice were treated with PBS, an isotype control human mAb, or mAb MPV467 24 hour (hrs) prior to hMPV infection or three days after hMPV infection in both prophylactic and treatment studies (FIG. 4). On day five, viral titers in the lungs of mice were determined by plaque assay. No virus was detectable in mice treated with MPV467 in either study, while virus was present in both PBS-treated and isotype-mAb-treated mice. No difference was observed between MPV467 and uninfected mice, and no difference was observed between PBS and isotype control mice in either study.

### Example 6

Structural Definition of the hMPV F-MPV467 Complex

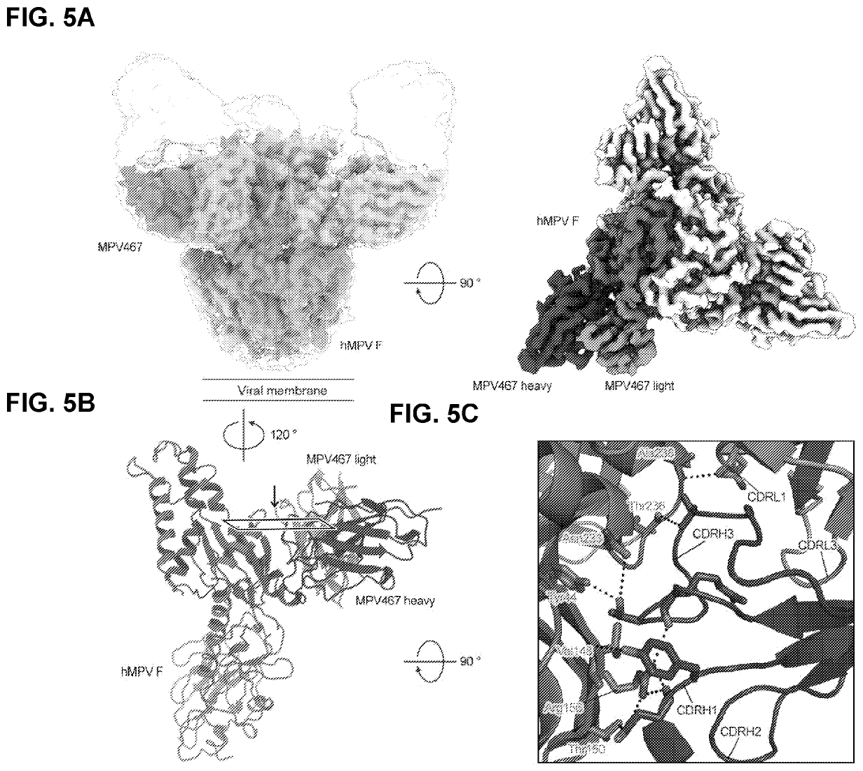

As MPV467 is the most potent hMPV F mAb described to date, is protective against and can treat hMPV infection, and targets an undefined epitope, we determined the structure of MPV467 in complex with pre-fusion hMPV F using cryo-electron microscopy (cryo-EM) to a global resolution of 3.3 Å (FIGS. 5A-5C). A final map for model building was generated by particle subtraction of the flexible Fab constant regions and sharpening via DeepEMhancer (Sanchez-Garcia et al,. Commun. Biol. 4, 874 (2021)). The structure revealed that MPV467 has an angle of approach directed down toward the viral membrane (FIGS. 5A, 5B). The heavy and light chains bury a surface area of 567 Å² and 322 Å², respectively, on hMPV F. The MPV467 epitope spans antigenic sites II and V, but also contacts a single residue in antigenic site III, Tyr44. The hMPV F site V residue Arg156 makes numerous interactions to MPV467 via hydrogen bonds to both CDRH1 and CDRH3 mainchain atoms as well as a salt bridge interaction with CDRH1 Asp31 (FIG. 5C). Additionally, CDRH1 Asp31 also forms a hydrogen bond with site V residue Thr150. The conformationally immobile antigenic site II is bound by the MPV467 CDRH3 through two hydrogen bond interactions with hMPV F Asn233 and Thr236. The only specific interaction between the light chain of MPV467 and hMPV F is through a malnchain hydrogen bond between CDRL1 Asn30 and hMPV F Ala238.

Humans have been exposed to hMPV for at least 70 years (van den Hoogen et al. Nat. Med. 7, 719-724 (2001)), yet the predominant epitopes on the hMPV F protein have remained elusive. Variable region sequence analysis showed the diversity of V_{H} and V_{K}/V_{L} gene usage in hMPV F-specific mAbs. Among these genes, V_{H}1-69 is shared by 4 out of 18 mAbs (MPV86, MPV414, MPV483, and MPV503), and three of them (except for MPV483) showed similar binning profiles that compete with MPE8 and MPV364 (FIGS. 2A, 2B), indicating they bind to the same hMPV F epitope possibly via similar binding patterns of the heavy chains. Similar correlations between binding epitopes and V gene usage were observed in light chains as well. Both mAbs MPV86 and 487 that share IGKV3-11 and mAbs MPV 414 and 503 that share IGKV3-15 bind to the MPV364 site, while mAbs MPV464 and 485 that share IGLV3-1 bind to DS7 site. mAbs MPV414 and MPV503 were identified from two different subjects using two different approaches, yet they have the same set of V_{H}/V_{K} genes, suggesting this pair of genes might be favored by hMPV F MPV364 site-specific mAbs.

The major antigenic sites on hMPV F were mapped. In addition to the known antigenic sites III, IV, the DS7-site, and the 66-87-site, site V was further characterized and a putative site II was identified. Similar to site III and site IV, the positions of both site V and site II resemble their counterparts on RSV F, suggesting the epitopes in these areas share structural features that can be recognized by human antibodies. However, no mAbs were found to bind the counterparts of RSV F site Ø on hMPV F, further suggesting mAbs to this epitope may be limited due to N-linked glycans on hMPV F at site Ø (Asn57 & Asn172) (Poor et al., PNAS 111, 2596-2605 (2014)).

The most potent mAb, MPV467, bound an epitope located across antigenic sites II and V. Structures of hMPV F in its pre- and post-fusion conformations reveal that it has structural homology with the related RSV F protein and neutralizing epitopes on RSV F can be expected to have counterparts on hMPV F. Structurally, antigenic sites Ø and V are pre-fusion specific and previously isolated RSV antibodies and structural studies have shown that antibodies targeting these regions tend to be highly potent neutralizers (Gilman et al., Sci. Immunol. 1, 1-12 (2016); Mousa et al., Nat. Microbiol. 2, 16271 (2017)). Here with the cryo-EM structure of mAb MPV467 in complex with hMPV F, it was determined that this mAb is one of the first site V-targeting antibodies discovered for hMPV F and that it does indeed share the characteristic of potent neutralization. MPV467 binds the beta hairpin (β3-β4) located within antigenic site V, which undergoes a large conformational change during the transition from pre-fusion to post-fusion. Without being bound by theory, this region of the MPV467 epitope is likely responsible for the potency of MPV467. While bound, the fusion protein cannot transition to the post-fusion conformation, which is essential for efficient viral infection. Additionally, the helix-turn-helix (α6-α7) located within antigenic site II that is bound by MPV467 does not undergo a conformational change between pre- and post-fusion states. The binding of residues in this region is likely why the ability of MPV467 to bind both conformational states was seen. Since only this part of the epitope is present in the post-fusion conformation, a drop in binding affinity was seen, compared to the pre-fusion conformation which includes the entire epitope.

An RSV/hMPV F cross-reactive mAb M1C7 (Xiao, et al., MAbs 11, 1415-1427 (2019)) was previously reported as a potent neutralizing mAb targeting site V. Similarly, an RSV F site V-specific mAb, hRSV90 (Mousa et al., Nat. Microbiol. 2, 16271 (2017)), also has a low IC₅₀ against RSV (4 ng/mL for RSV A, 10 ng/mL for RSV B) indicating site V is a vulnerable area that is favored by ultrapotent neutralizing mAbs for Pneumoviruses. The site V epitope of both RSV F and hMPV F is located on the N-terminus of the F₁ subunit, closely following the fusion peptide, which is buried in the center of the trimeric pre-fusion F protein. Hence, the conformational change of the α3 helix and β3-β4 hairpin in site V is vital to expose the fusion peptide and initiate the fusion process. Antibodies targeting site V likely lock the fusion peptide and prevent the formation of the long helical bundles that are present in the post-fusion conformation. However, based on current findings, the frequency of hMPV site V-specific antibodies is relatively low compared to antibodies targeting site III, site IV, and DS7 site. Therefore, boosting antigenic site V targeting antibodies could be boosted in hMPV F-based vaccines.

### Example 7

### Materials and Methods for Examples 1-6

Blood draws and PBMC isolation: After obtaining informed consent, 90 mL of blood was drawn by venipuncture into 9 heparin-coated tubes, and 10 mL of blood was collected into a serum separator tube. Peripheral blood mononuclear cells (PBMCs) were isolated from human donor blood samples using Ficoll-Histopaque density gradient centrifugation, and PBMCs were frozen in the liquid nitrogen vapor phase until further use.

Production and synthesis of recombinant hMPV F protein: hMPV A1, A2, B1, B2 F and hMPV B2F-GCN4 recombinant proteins were synthesized from the plasmids obtained from GENSCRIPT® cloned into pcDNA3.1+ vector. They were expanded by transforming them into DH5a cells against ampicillin (Thermo Scientific) resistance 100 μg/ml. The plasmids were purified using E.N.Z.A. plasmid maxiprep kit (Omega BioTek) following the manufacturer's instructions. 1 mg of plasmid was mixed with 4 mg of polyethyleneimine (PEI; PolySciences Inc.) in OPTI-MEM® cell culture medium (Thermo Scientific) and incubated for 30 minutes. This was followed by addition of the DNA-PEI mixture to 10⁶ cells/ml 293 cells in Freestyle 293 expression medium (Thermo Fischer). After 5 days of incubation, the cultures were centrifuged at 6000 g to pellet the cells. The supernatant was filtered through a 0.45 μm sterile filter. The recombinant proteins were purified directly by affinity chromatography, HisTrap Excel columns (GE Healthcare Life Sciences). Prior to loading the supernatant onto the column, it was washed with 5 column volumes (CV) of a wash buffer containing 20 mM Tris-HCl (pH 7.5), 500 mM NaCl and 20 mM Imidazole. After passing the supernatant through the column, it was washed with the same wash buffer (5 CV) to reduce non-specific binding and finally eluted with buffer containing 20 mM Tris-HCl pH 7.5, 500 mM NaCl and 250 mM Imidazole. After elution, the proteins were concentrated with AMICON® Ultra-15 centrifugal units with a molecular cut-off of 30 KDa (Sigma).

Trypsinization of hMPV F: In order to obtain trimeric hMPV F, TPCK (L-1-tosylamido-2-phenylethyl chloromethyl ketone)-trypsin (Thermo Scientific) was dissolved in double-distilled water (ddH₂O) at 2 mg/mL. hMPV B2 F obtained, was incubated with 5 TAME (p-toluene-sulfonyl-L-arginine methyl ester) units/mg of TPCK-trypsin for 1 hr at 37° C. The trimeric and monomeric fractions of hMPV F were separate by size exclusion chromatography on a SUPERDEX® S200, 16/600 column (GE Healthcare Life Sciences) in column buffer (50 mM Tris pH 7.5, and 100 mM NaCl). Both fractions were separated by their unique elution profiles. Once separated, they were concentrated as mentioned earlier. Post-fusion hMPV F was obtained by heating the pooled trimeric fractions at 55° C. for 20 minutes on a water bath to induce post-fusion conformation (Jiachen et al., J. Virol. 95, e00593-21 (2021)).

Generation of hMPV F-specific hybridomas: For hybridoma generation, 10 million peripheral blood mononuclear cells purified from the blood of human donors were mixed with 8 million previously frozen and gamma irradiated NIH 3T3 cells modified to express human CD40L, human interleukin-21 (IL-21), and human BAFF (Bar-Peled et al., J. Virol. 93, e00342-19 (2019)) in 80 mL STEMCELL® medium A (STEMCELL® Technologies) containing 6.3 μg/mL of CpG (phosphorothioate-modified oligodeoxynucleotide ZOEZOEZZZZZOEEZOEZZZT; Invitrogen nd 1 μg/mL of cyclosporine (Sigma). The mixture of cells was plated in four 96-well plates at 200 μl per well in StemCell medium A. After 6 days, culture supernatants were screened by ELISA for binding to recombinant hMPV B2 F protein, and cells from positive wells were electrofused as previously described (Bar-Peled et al., J. Virol. 93, e00342-19 (2019)). Cells from each cuvette were resuspended in 20 mL STEMCELL® medium A containing 1×HAT (hypoxanthine-aminopterin-thymidine; Sigma-Aldrich), 0.2×HT (hypoxanthine-thymidine; Corning), and 0.3 μg/mL ouabain (Thermo Fisher Scientific) and plated at 50 μl per well in a 384-well plate. After 7 days, cells were fed with 25 μl of StemCell medium A. The supernatant of hybridomas were screened after 2 weeks for antibody production by ELISA, and cells from wells with reactive supernatants were expanded to 48-well plates for 1 week in 0.5 mL of STEMCELL® medium E (STEMCELL® Technologies), before being screened again by ELISA. Positive hybridomas were then subjected to single-cell fluorescence-activated sorting into 384-well plates containing 75% of STEMCELL® medium A plus 25% of STEMCELL® medium E. Two weeks after cell sorting, hybridomas were screened by ELISA before further expansion of wells containing hMPV F-specific hybridomas.

RT-PCR for hybridoma mAb variable gamma chain and variable light chain: RNA was isolated from expanded hybridoma cells using the ENZA total RNA kit (Omega BioTek) according to the manufacturer's protocol. A High-Capacity cDNA Reverse Transcription Kit (Applied Biosystems) was used for cDNA synthesis. Three separate sets of primer mixes were used for nested PCR to amplify the variable regions of gamma, kappa, and lambda chains (Tiller et al., J. Immunol. Methods 329, 112-124 (2008)). The products from the second PCR were analyzed by agarose gel electrophoresis and purified PCR products (ENZA cycle pure kit; Omega Biotek) were submitted to Genewiz for sequencing. Sequences were analyzed using IMGT/V-Quest (Brochet et al., Nucleic Acids Res. 36, 503-508 (2008)).

Antigen-specific single B cell sorting and expression of recombinant mAbs: Ten million human PBMCs were washed twice with FACS buffer and then resuspended with 1 mL FACS buffer. The cells were treated with 5% Fc receptor blocker (BIOLEGEND®) for 30 minutes and then stained with following antibodies: human CD19-APC, human IgM-FITC, human IgD-FITC, GHOST DYE™ Red 710, and PE/BV605-streptavidin conjugated hMPV B2 F. Antigen specific B cells were gated with CD19+/IgM-/IgD-/Ghost dye-/PE+/BV605+ and sorted in catch buffer B (Qiagen TCL Buffer+1% beta mercaptoethanol) by one cell per well in a 96 well plate. Sorted cells were flash frozen and stored in −80° C. until they were used for RNA extraction. The RNA was extracted with Agencort RNACLEAN® XP kit, SPRI Beads (Beckman Coulter) and immediately reverse transcribed to cDNA with SUPERSCRIPT® IV Synthesis System (ThermoFisher). The variable region sequences of IgG heavy/light chains were determined by nested PCR as described above. Based on the usage of V/D/J gene alleles, cloning PCR primers were picked for cloning PCR with the first PCR products as the template. Purified cloning PCR products of heavy/light chains were cloned into expression vectors (AbVec-hIgG1, AbVec-hIgKappa, and pBR322-based Ig-lambda expression vector) and the plasmids were sent to Genewiz for sequencing. After confirming all the sequences are correct, the HC/LC plasmids were transformed into DH5a for plasmid maxiprep. Recombinant mAbs were expressed by transfecting 293 cells with HC/LC plasmids and purified from culture supernatant with Protein G column (Cytiva).

Enzyme-linked immunosorbent assay for binding to hMPV F protein: The 384 well plates (catalog number 781162; Greiner BIO-ONE®) used for ELISA were coated with 2 μg/ml (in PBS) of the recombinant protein (antigen) and incubated overnight at 4° C. This was accompanied by washing the plates once, with water followed by blocking them for 1 hr at room temperature with Block buffer comprising of 2% milk supplemented with 2% goat serum in PBS and 0.05% Tween 20 (PBS-T). The plates were washed again thrice with PBS-T. 25 μl of the serially diluted primary antibodies were added to the wells and incubated for an hour at room temperature followed by three washes with PBS-T. Goat anti-human IgG Fc secondary antibody (Southern Biotech) diluted in block buffer (1:4000) was next applied to the wells and incubated again at room temperature for an hour. The plates were washed again with PBS-T thrice and 25 μl of PNPP (p-nitrophenyl phosphate) diluted to a concentration of 1 mg/ml in a buffer containing 1M Tris base and 0.5 mM Magnesium Chloride having a pH of 9.8 was added. Prior to reading the absorbance at 405 nm on a Bio Tek plate reader, the plates were incubated one last time at room temperature for 1 hr. The binding assay data was analyzed on GraphPad Prism using a nonlinear regression curve fit and log(agonist)-versus-response function for calculating the EC₅₀ values.

hMPV plaque neutralization experiments: LLC-MK2 cells used for this experiment were grown in Opti-MEM I (Thermo Fischer Scientific) that was supplemented with 2% fetal bovine serum in T225 cell culture flasks (catalog number 82050-870) at 37° C. in a CO₂ incubator. Two days before beginning the neutralization assay, 40,000 cells/well were plated on 24-well plates. Serially diluted sterile-filtered mAbs isolated from hybridoma supernatants were added to the suspension of either of hMPV strains, CAN/97-83 and TN/93-32 in equal amounts (1:1) and incubated for 1 hour on the day of the experiment. This was followed by addition of 50 μl of the virus-antibody mixture to the LLC-MK2 cells after washing of the excess FBS from the OPTI-MEM media with PBS three times. The mixture was incubated at room temperature for an hour with constant rocking. The cells were next overlaid with 0.75% methylcellulose dissolved in Opti-MEM I supplemented with 5 g/ml of trypsin-EDTA and 100 μg/ml of CaCl₂. The cells were incubated for 4 days and fixed with 10% neutral buffered formalin. Cell monolayers were next blocked with block buffer comprising of 2% nonfat milk supplemented with 2% goat serum in PBS-T for an hour. Next, the plates were washed thrice with water and 200 μl of MPV 364 was added to a final concentration of 1 μg/ml (1:1000 dilution) in blocking solution. The plates were then washed three times with water and 200 μl of goat anti-human IgG HRP secondary antibody (Southern Biotech) diluted to a ratio of 1:2000 in block buffer was added and incubated for 1 hr at room temperature followed by an hour of incubation. Plates were washed again with water five times and 200 μl of TRUEBLUE® peroxidase substrate (SERACARE®) was added to each well. The plates were incubated for 20-30 minutes until the plaques were clearly visible. Plaques were counted manually under a microscope and compared to the virus-only control. GraphPad Prism was used to calculate the IC₅₀ values using a nonlinear regression curve fit and the log(inhibitor)-versus-response function.

Epitope Binning: 100 g/ml of the his-tagged hMPV B2F (not trypsin treated) protein was immobilized on anti-penta-His biosensor tips (Forte′ Biosciences) for 120 s after obtaining the initial baseline in running buffer (PBS, 0.5% BSA, 0.05% Tween 20 and 0.04% thimerosal). Base line was measured again with the tips immersed in wells containing 100 μg/ml of the primary antibody for 300 s. This was followed by immersing the biosensor tips again for 300 s in the secondary antibody at 100 g/ml. Binding of the second mAb in the presence of the first mAb as determined by comparing the maximal signal of the second mAb after the first mAb was added to the maximum signal of the second mAb alone. Non-competing mAbs were those whose binding was greater than or equal to 70% of the uncompeted binding. Between 30% and 60% was considered intermediate binding and anything lower that 30% was considering as competing for the same site.

Antibody-dependent phagocytic activity of mAbs: To measure antibody-dependent phagocytic activity, 2×10⁹ 1-μm Neutravidin-coated yellow-green FLUOSPHERES® (Invitrogen #F8776) were resuspended in 1 mL of 0.1% PBS. The FLUOSPHERES® were then centrifuged at 5000 rpm for 15 minutes, 900 μL supernatant was removed, and the FLUOSPHERES® were resuspended with 900 μL of 0.1% PBS. This process was repeated for a second wash, then the FLUOSPHERES® were resuspended with 20 μg of biotinylated hMPV B2 F protein. The FLUOSPHERES® were then incubated overnight at 4° C., protected from light, with end-to-end rocking. Next, hMPV F-specific antibodies were diluted in complete RPMI media (cRPMI, RPMI+10% FBS) to a final concentration of 1 μg/mL in a U-bottom 96-well plate. Then, 20 μL of antibody dilution was transferred into a clean F-bottom 96-well plate, and 10 μL of FLUOSPHERES® were added with the antibody followed by a 2 hr incubation at 37° C. for opsonization. After 1.5 hr, THP-1 cells were centrifuged at 200×g for 5 min, washed once with PBS, then resuspended in culture medium (RPMI & 10% FBS) at a concentration of 5×10⁵ cells/mL. Then, 200 μL of cells were added to each well and incubated at 37° C. with 5% CO₂ while shaking for 6 hr. Once the incubation finished, the plate was then centrifuged at 2000 rpm for 5 min. Then, 100 μL was pipetted out of each well and replaced with 100 μL of cold 4% paraformaldehyde to fix the cells. The plate was then left at room temperature for 20 min, protected from light. The plate was then stored at 4° C. in the dark. Cells were then analyzed with a NOVOCYTE® QUANTEON® flow cytometer. The percentage of fluorescent beads containing THP-1 cells in each sample (% phagocytosis) was used to calculate % increase vs. no mAb control. The phagocytic scores were calculated as previously described,⁵⁰ (geometric mean intensity−the geometric mean intensity of the no mAb control)×% phagocytosis.

Animal studies: BALB/c mice (6 to 8 weeks old; The Jackson Laboratory) were randomly selected to each group that contains 5 males and 5 females. All the mice were pre-bleed before the study to verify the mice were not pre-exposed to hMPV by ELISA. Each mouse was intranasally infected with hMPV TN/93-32 (5×10⁵ PFU) and euthanized five days post-infection. Mice were i.p. injected with PBS/MPV467/control antibodies (10 mg/kg) 24 hours prior to infection (prophylaxis) or three days post-infection (treatment). At the end point, serum was collected for ELISA to determine the presence of mAb MPV467, the lungs were collected and homogenized to determine the viral load through immunostaining as described above.

Recombinant protein production for cryo-EM studies: The prefusion hMPV F construct DS-CavEs2-IPDS (hMPV F Al NL/1/00, residues 1-490) used for structural studies includes the previously described G294E, A185P, L219K, V231I, E453Q and furin cleavage site substitutions (Battles et al., Nat. Commun. 8, 1528 (2017)). Disulfide substitutions included are L110C/N322C, T127C/N153C, A140C/A147C and T365C/V463C (Hsieh et al, in press) as well as an interprotomer disulfide at V84C/A249C (Stewart-Jones et al., Proc. Natl. Acad. Sci. 118, e2106196118 (2021)). DS-CavEs2-IPDS was cloned into the mammalian expression vector paH with a C-terminal “GGGS” linker sequence followed by the T4 fibritin trimerization motif “foldon” (Efimov et al., J. Mol. Biol. 242, 470-486 (1994); Miroshnikov et al., Protein Eng. 11, 329-332 (1998)) an HRV3C protease site, an 8×His tag, and a Strep-Tagll (Battles et al., Nat. Commun. 8, 1528 (2017)). Transient co-transfection of FreeStyle 293F cells (ThermoFisher) at a 4:1 ratio of DS-CavEs2-IPDS:furin-expressing plasmids by polyethylenimine (PEI) was used for protein expression. Kifunensine and Pluronic F-68 (Gibco) were introduced three hours post-transfection to a final concentration of 5 μM and 0.1% (v/v), respectively. Six days post-transfection, STREP-TACTIN® Sepharose resin (IBA) was used to purify soluble protein from the cell supernatant which had been filtered and buffer-exchanged into PBS by tangential flow filtration. Buffer containing 100 mM Tris pH 8.0, 150 mM NaCl, 1 mM EDTA and 2.5 mM desthiobiotin was used to elute the strep-tagged protein. After concentrating the protein using a 30 kDa molecular weight cut-off AMICON® Ultra-15 centrifugal filter unit (Millipore) the protein was further purified by size-exclusion chromatography using a SUPEROSE® 6 Increase 10/300 column (GE Healthcare) in 2 mM Tris pH 8.0, 200 mM NaCl, and 0.02% NaN₃ running buffer.

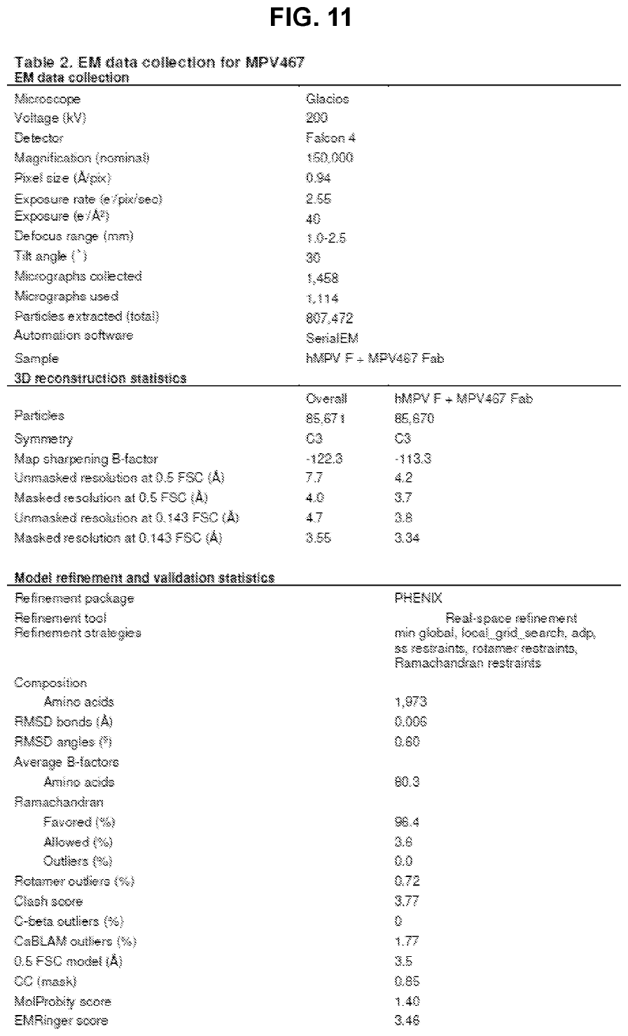

Cryo-EM Sample Preparation and Data Collection: Purified DS-CavEs2-IPDS was combined with a 1.5-fold molar excess of MPV467 Fab incubated at room temperature for 10 minutes before being moved to ice. Just before freezing, sample was diluted to a concentration of 0.66 mg/mL hMPV F in 2 mM Tris pH 8.0, 200 mM NaCl, and 0.02% NaN₃ buffer. Then 1 μL of 0.5% amphipol A8-35 was combined with 10 μL of diluted sample and 4 μL of this sample was applied to a gold 1.2/1.3 300 mesh grid (Protochips Au-Flat) that had been plasma cleaned for 180 seconds using a SOLARUS® 950 plasma cleaner (Gatan) with a 4:1 ratio of O₂/H₂. Grids were plunge-frozen using a VITROBOT® Mark IV (Thermo Fisher) with a 10° C., 100% humidity chamber. Blotting settings were 5 seconds of wait followed by 4 seconds of blotting with −2 force before plunging into nitrogen-cooled liquid ethane. Using a GLACIOS® (Thermo Scientific) equipped with a Falcon 4 direct electron detector (Thermo Scientific), a single grid was imaged to collect a total of 1,458 images. Data were collected at a 30° tilt with magnification of 150,000× corresponding to a calibrated pixel size of 0.94 Å/pix and a total exposure of 40 e⁻/Å². Data collection statistics are listed in FIG. 11.

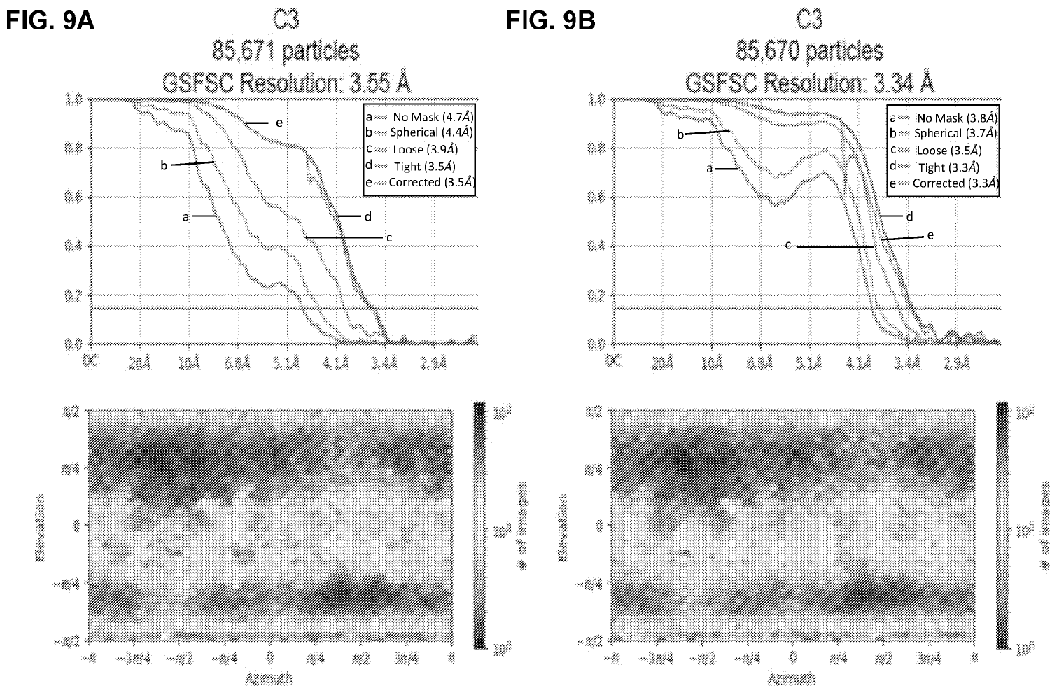

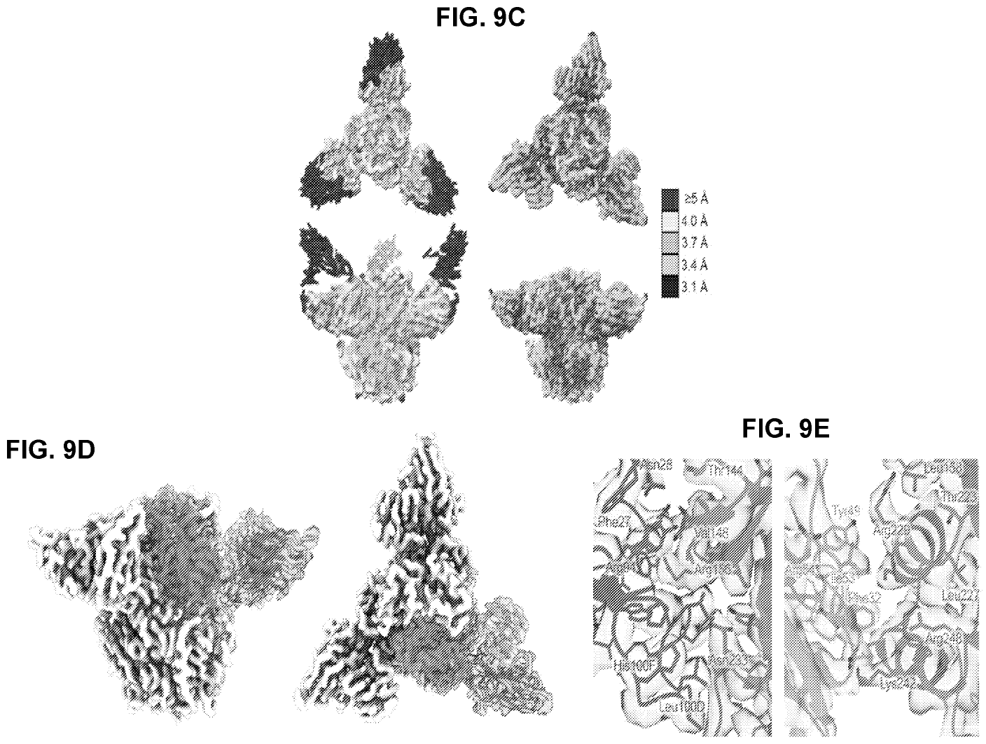

Cryo-EM data processing Micrographs were corrected for gain reference and imported into CRYOSPARC® Live v3.2.0 for initial data processing: motion correction, defocus estimation, micrograph curation, particle picking and extraction, and particle curation through iterative streaming 2D class averaging. 2D averages were used to generate templates and template-based particle picking was carried out. Curated particles were exported to CRYOSPARC® v3.2 for further processing via rounds of 2D classification, ab initio reconstruction, heterogeneous refinement, homogenous refinement, and non-uniform homogenous refinement using C3 symmetry. Masking and particle subtraction were used for further non-uniform refinement. Finally, the particle-subtracted non-uniform refinement map was sharpened using DeepEMhancer (Sanchez-Garcia et al,. Commun. Biol. 4, 874 (2021); Cianfrocco, et al., COSMIC2: A Science Gateway for Cryo-Electron Microscopy Structure Determination, in Proceedings of the Practice and Experience in Advanced Research Computing 2017 on Sustainability, Success and Impact (Association for Computing Machinery, 2017). doi:10.1145/3093338.3093390). EM processing workflows are shown in FIG. 8, and EM validation results are shown in FIG. 9. For model building, an initial hMPV F model was generated from PDB ID: 5WBO and the crystal structure of MPV467 Fab which were used to dock into the cryoEM maps using UCSF ChimeraX (Pettersen et al., Protein Sci. 30, 70-82 (2021)). Models were built further and iteratively refined using a combination of Coot (Emsley et al., Acta Crystallogr. D. Biol. Crystallogr. 66, 486-501 (2010)), PHENIX (Liebschner et al., Acta Crystallogr. Sect. D, Struct. Biol. 75, 861-877 (2019)), and ISOLDE (Croll, Acta Crystallogr. Sect. D 74, 519-530 (2018)). Model statistics are shown in FIG. 11.

### Example 8

Human Metapneumovirus (hMPV) Infection and MPV467 Treatment in Immunocompromised Cotton Rats Sigmodon hispidus

The cotton rat Sigmodon hispidus is an established model of respiratory virus infections, including those caused by RSV, influenza, adenoviruses, parainfluenza, rhinovirus, and enterovirus (Blanco et al., J Antivir Antiretrovir 2014; 6:40-42; Patent et al., PLoS One 2016; 11(11):e0166336). hMPV infection has been modeled in cotton rats S. hispidus shortly after the virus discovery (Hamelin et al., J Virol 2005; 79(14):8894-903; Williams et al., J Virol 2005; 79(17):10944-51; Wyde et al., Antiviral Res 2005; 66(1):57-66; Yim et al., Vaccine 2007; 25(27):5034-40). Cotton rats S. hispidus immunosuppressed by cyclophosphamide were infected with hMPV and viral replication and pulmonary inflammation in these animals was compared to that in normal hMPV-infected S. hispidus. Efficacy of prophylactic and therapeutic administration of the anti-hMPV antibody MPV467 was also evaluated. Immunosuppressed animals had higher pulmonary and nasal titers of hMPV on day 5 post-infection compared to normal animals and large amounts of hMPV were still present in the respiratory tract of immunosuppressed animals on days 7 and 9 post-infection, indicating prolonged viral replication. Immunosuppression was accompanied by reduced pulmonary histopathology in hMPV-infected cotton rats compared to normal animals, however, a delayed increase in pathology and pulmonary cytokine/chemokine expression was seen in immunosuppressed cotton rats. Prophylactic and therapeutic MPV467 treatments protected both upper and lower respiratory tracts against hMPV infection. Lung pathology and pulmonary expression of IP-10 and MIP-1α mRNA were reduced by therapeutic MPV467 administration. The results are provided below.

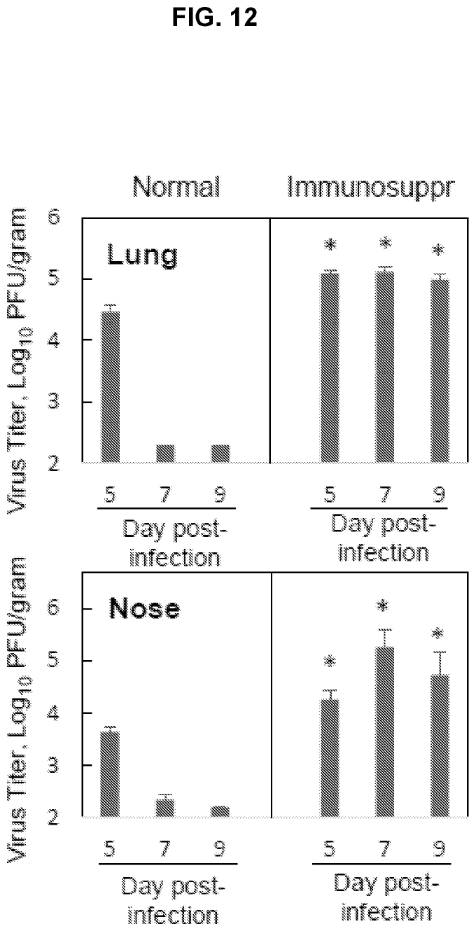

Immunosuppression results in increased hMPV replication and delayed viral clearance in cotton rats: Immunosuppression can be associated with more severe disease and prolonged replication of respiratory viruses in affected individuals (Welliver, J Pediatr 2003; 143(5 Suppl):S12-7; Lion. Clin Microbiol Rev 2014; 27(3):441-62; Hijano et al., Front Microbiol 2018; 9:3097). To assess hMPV replication and clearance in cyclophosphamide-immunosuppressed cotton rats, animals were infected with 10⁵ PFU of hMPV and viral replication was assessed at several time points after infection. Lung and nose samples were collected for viral titration on day 5 post-infection, the time of peak viral replication in normal cotton rats, on day 7, the time when virus is cleared from the lungs of infected cotton rats, and on day 9, an additional delayed time point. Comparison was made to normal age-matched animals infected in parallel with the immunosuppressed animals and sacrificed on the same days. As expected, in normal animals, hMPV was cleared from the lungs and essentially cleared from the nose of infected normal cotton rats by day 7 post-infection and was not detectable in either lower or upper respiratory tracts on day 9 (FIG. 12). In contrast, immunosuppressed cotton rats showed high levels of hMPV in the lungs and nose on all three days. The amount of hMPV present in the lungs and nose of infected animals on day 5 post-infection was significantly higher than what was detected in normal cotton rats sacrificed at the same time. Overall, these results indicate that hMPV can replicate to higher titers in immunosuppressed cotton rats and that the clearance of virus is significantly delayed by immunosuppression.

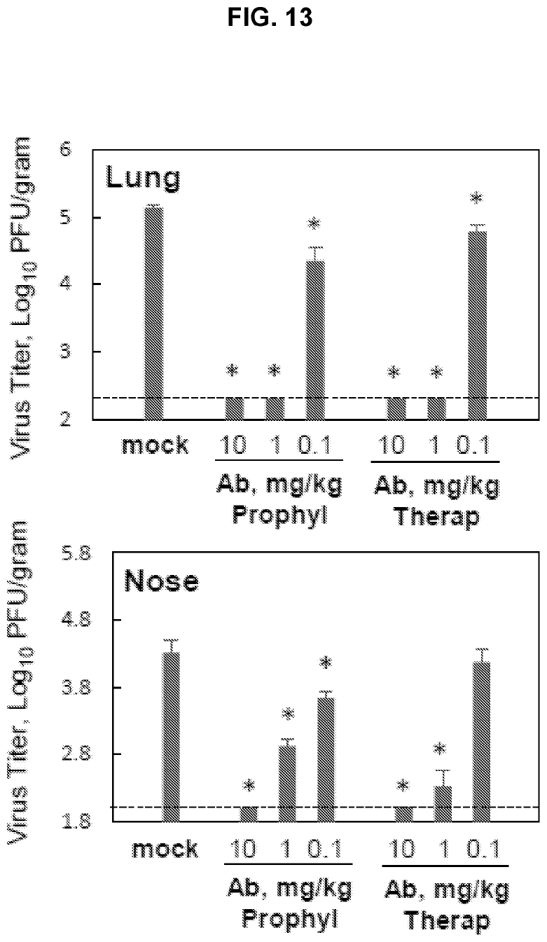

Prophylactic or therapeutic treatment with anti-hMPV antibody reduces hMPV load in immunosuppressed cotton rats in a dose-dependent manner: Monoclonal antibodies targeting viral surface proteins are among the most efficient therapeutics and prophylactics for viral infections used today (Pantaleo et al., Nature Reviews Drug Discovery 2022; 21:676-696). The prophylactic and therapeutic efficacy of MPV467 was assessed in normal and immunosuppressed cotton rats infected with hMPV and sacrificed on day 5 post-infection (FIG. 13). Three different doses of MPV467 were tested: 0.1, 1, and 10 mg/kg one day before or three days after infection. Results of the testing showed that both prophylactic and therapeutic treatments with MPV467 were effective at reducing hMPV replication in the lungs and nose of infected animals. The two highest doses of MPV467 tested, 10 and 1 mg/kg, given either before or after infection, completely protected the lungs of infected animals (undetectable viral replication). The lowest dose of MPV467 tested, 0.1 mg/kg, caused a modest, but statistically-significant reduction in lung hMPV replication, with the effect being slightly more pronounced for prophylactic treatment. In the nose, significant reduction of hMPV load was afforded by all three doses of MPV467 tested when administered prophylactically, and by the two highest doses when given therapeutically. Overall, these results indicate that MPV467 has a strong dose-dependent antiviral activity in immunosuppressed cotton rats.

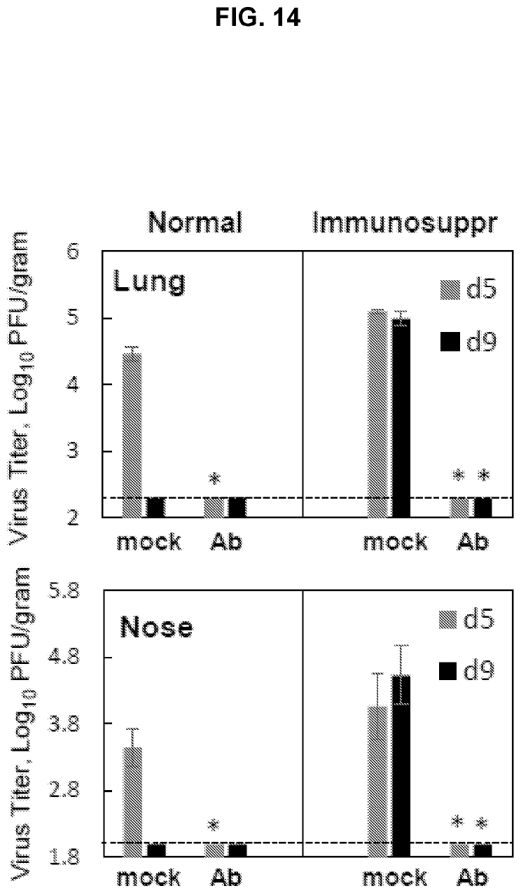

MPV467 therapy ameliorates delayed hMPV clearance in immunosuppressed cotton rats: Once antiviral efficacy of MPV467 was ascertained in immunosuppressed cotton rats by analyzing samples collected at the peak time of viral replication in the lung, it was determined whether therapeutic administration of antibodies would be able to combat delayed viral clearance in the model. To address this question, hMPV-infected immunosuppressed animals were treated with 10 mg/kg MPV467 three and seven days after infection and sacrificed on day nine post-infection for analysis of viral load in the lungs and nose. Normal cotton rats were infected and treated once, on day three. Replication on day five was assessed again in hMPV-infected animals (normal and immunosuppressed) treated with 10 mg/kg MPV467 (or mock-treated) three days after infection. MPV467 was highly efficacious in normal cotton rats, reducing viral replication to undetectable level in day five samples (FIG. 14). MPV467 was also highly efficacious in immunosuppressed animals. Mock-treated hMPV-infected immunosuppressed animals had comparable amounts of hMPV detectable on days five and nine in the lungs and noses, while animals treated with MPV467 had no detectable hMPV in either upper or lower respiratory tracts on both days of analysis.

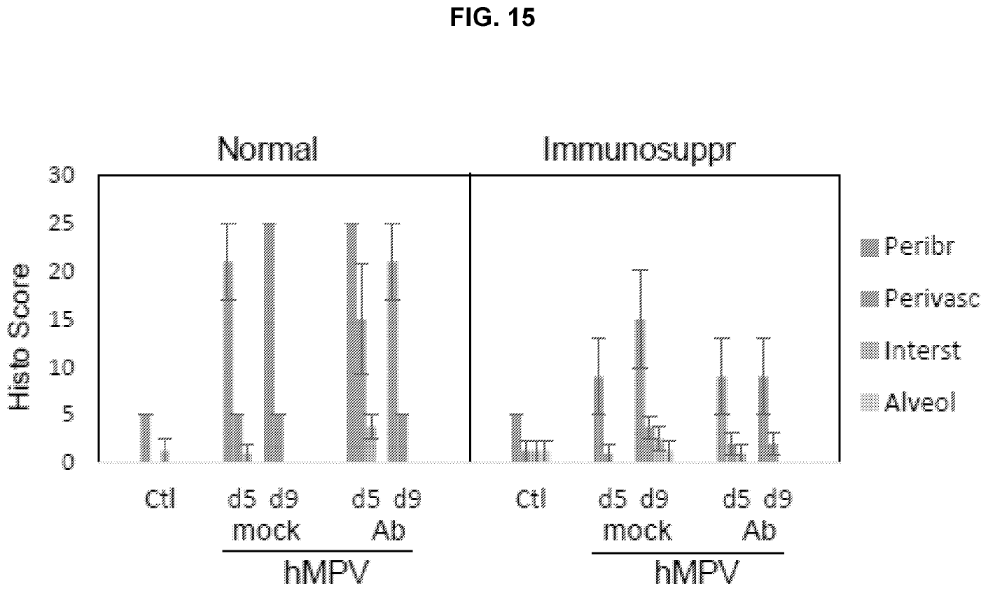

Effect of antibody therapy on lung histopathology and cytokine/chemokine expression in immunosuppressed cotton rats: Pulmonary histopathology, one of the markers of inflammatory response to respiratory infection in the cotton rat model, was used to assess differences in lung response to hMPV in normal and immunosuppressed animals in the presence or absence of antibody treatment. Cotton rats were infected with hMPV and sacrificed on days five and nine post-infection for analysis of peribronchiolitis, perivasculitis, interstitial inflammation, and alveolitis. Normal, hMPV-infected animals had moderate level of pathology characterized predominantly by peribronchiolitis and some perivasculitis (FIG. 15). The extent of pathology was largely comparable between days five and nine. Antibody treatment caused a moderate increase in perivasculitis in normal hMPV-infected cotton rats. Immunosuppressed cotton rats infected with hMPV developed reduced peribronchiolitis compared to normal animals. Pulmonary histopathology in hMPV-infected immunosuppressed animals was slightly higher on day nine compared to day five, with interstitial inflammation and alveolitis becoming evident. Effect of therapeutic antibody treatment on lung pathology was evaluated for MPV467 administered in 10 mg/kg dose. A decrease in pulmonary histopathology in hMPV-infected antibody-treated immunosuppressed animals on day nine post-infection was seen compared to hMPV-infected mock-treated immunosuppressed animals.

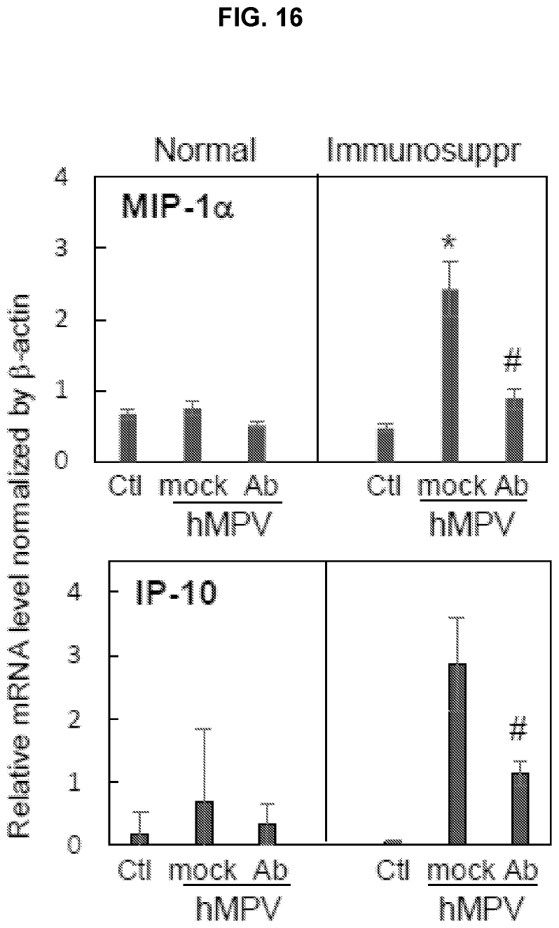

Expression of lung cytokines/chemokines is another marker of pulmonary inflammatory response to infections in the cotton rat model. To determine if a decrease in interstitial inflammation and alveolitis seen on day nine in immunosuppressed hMPV-infected animals treated with MPV467 was associated with altered pulmonary cytokine/chemokine expression, levels of pulmonary MIP-1α and IP-10 (mediators linked to lung injury and immune dysfunction (Ichikawa et al., Am J Respir Crit Care Med 2013; 187(1):65-77; Shanley et al., J Immunol 1995; 154(9):4793-802; Kameda et al., PLoS One 2020; 15(11):e0241719; Smith et al., J Immunol 1994; 153(10):4704-12) were measured. Immunosuppressed animals had elevated expression of MIP-1α and IP-10 mRNA compared to normal animals (FIG. 16). Expression of both mediators in immunosuppressed animals was significantly reduced by antibody treatment.

Normal (un-manipulated) cotton rats S. hispidus are susceptible to hMPV infection in the upper and lower respiratory tracts, with infection largely resolving within a week.²⁰⁻²¹ This is different from persistent hMPV infection that has been reported for regular BALB/c mice, where hMPV replication can sometimes last for weeks to months (Alvarez et al., J Virol 2004; 78:14003-14011; Alvarez et al., J Virol 2005; 79(10):5971-8; Moe et al., J Infect Dis 2017; 216(1):110-116). The self-limiting hMPV infection in un-manipulated cotton rats resembles the self-limiting hMPV infection in healthy humans (Moe et al., J Infect Dis 2017; 216(1):110-116; Ebihara et al., J Clin Microbiol 2004; 42:126-32) and was a beneficial feature that allowed for assessment of a potential viral clearance defect that could be caused by immunosuppression.

Similar to what has been reported for immunocompromised humans (Spahr et al., Open Forum Infect Dis 2018; 5(5):ofy077; Chu et al., J Pediatric Infect Dis Soc 2014; 3(4):286-93; Debiaggi et al., J Infect Dis 2006; 194(4):474-8; Debiaggi et al., New Microbiol 2007; 30(3):255-8), hMPV replication was significantly prolonged in immunosuppressed cotton rats, confirming a delayed viral clearance under conditions of suppressed immunity. Delayed viral clearance, in general, may impact lung function by direct cytopathic effect of prolonged virus replication, or by an indirect effect on lung inflammation. In this work, it was noted that interstitial inflammation and alveolitis were slightly increased in hMPV-infected immunosuppressed cotton rats later in infection compared to the earlier time corresponding to peak viral replication in the lung. Concurrently, expression of pulmonary cytokines/chemokines IP-10 and MIP-la in immunosuppressed animals this late in infection surpassed that detected in normal cotton rats infected with hMPV. The increase in specific parameters of pulmonary inflammation later in infection of immunosuppressed animals was similar to that seen before for RSV (Boukhvaolova et al., Bone Marrow Transplant 2016; 51(1): 119-26), with the exception that no cytokines were measured for the RSV model and no detectable epithelial damage was seen in the lungs of hMPV-infected animals. Increased levels of IP-10 and MIP-1α late in hMPV infection in immunosuppressed animals may have a detrimental effect on the lung. Both molecules were shown to promote development of lung injury of viral and non-viral origin through neutrophil-mediated mechanisms ((Ichikawa et al., Am J Respir Crit Care Med 2013; 187(1):65-77; Shanley et al., J Immunol 1995; 154(9):4793-802), and both molecules have been linked to the development of autoimmunity and pulmonary fibrosis (Kameda et al., PLoS One 2020; 15(11):e0241719; Smith et al., J Immunol 1994; 153(10):4704-12).

Therapeutic administration of anti-hMPV antibody MPV467 resulted in ablation of hMPV replication in immunosuppressed cotton rats, and it also reduced pulmonary pathology and cytokine/chemokine expression in the lungs of immunosuppressed animals. This combined suppression of viral replication and pulmonary inflammation by therapeutically-administered antiviral antibody is similar to the effect seen in the RSV-infected immunosuppressed cotton rats treated with anti-RSV Ig (Boukhvaolova et al., Bone Marrow Transplant 2016; 51(1): 119-26). For RSV, the ability of therapeutic antibody treatment to reduce lung inflammation in immunosuppressed animals contrasted with the lack of similar efficacy of antiviral antibodies when used alone in normal, non-immunosuppressed cotton rats (Prince et al., J Infect Dis 2000; 182:1326-1330; Boukhvalova et al., J Infect Dis 2007; 195(4):511-8), or in humans (Rodriguez et al., Pediatrics 1997; 100(6):937-42; Rodriguez et al., Pediatrics 1997; 99(3):454-61).

### Example 9

### Materials and Methods for Example 8

Reagents: Cyclophosphamide for injection (20 mg/ml USP, Baxter) was obtained from Blue Door Pharma.

Viruses and viral assays: The hMPV strain TN/94-49/A2 recovered from specimens collected in the Vanderbilt Vaccine Clinic (Williams et al., N Engl J Med 2004: 350(5):443-50; Williams et al., J Infect Dis 2006; 193(3):387-95), was grown on LLC-MK2 cells in minimal essential medium supplemented with 0.2% glucose, 0.1% bovine serum albumin, 0.0002% trypsin, and 1% gentamicin. A single pool of hMPV (3×10⁶ pfu/ml) was used for the studies described herein.

Animals and animal studies: Inbred S. hispidus cotton rats were obtained from a colony maintained at Sigmovir Biosystems, Inc. Six- to eight-week old male and female cotton rats were used for the studies. Animals were housed in large polycarbonate cages and were fed a standard diet of rodent chow and water. The colony was monitored for antibodies to adventitious respiratory viruses and other common rodent pathogens and no such antibodies were found (VRL Test 80221-RAT 1 Ab profile: Carbacillus CARB, Toolan's H-1 virus, Kilham Rat virus KRV, Mycoplasma pulmonis, Parvovirus generic, Pneumonia virus PVM, RCV/SDA, Sendai virus). hMPV infection in immunocompromised animals and efficacy of MPV467 treatment was verified in two consecutive experiments. Sample size of four to five animals per group was chosen based on results of previous experiments, as allowing detection of statistically-significant differences between groups. Comparison between groups was run using Student t-test for unpaired data with unequal variance (KaleidaGraph). Unless indicated, samples were not blinded prior to analysis.

Immunosuppression was induced in cotton rats by repeated treatment with cyclophosphamide (CY) based on a previously disclosed method (Boukhvalova et al., Bone Marrow Transplant 2016; 51(1):119-26). In brief, 50 mg/kg of CY solution was administered intramuscularly (i.m.) as 250 μl/100 g animal for 18 days on a Monday-Wednesday-Friday schedule. At the end of this period, whole blood was collected to verify the decline in total white blood cell and lymphocyte counts. CY treatment was continued until the end of the study. Twenty one days after the start of CY treatment, animals were infected intranasally (i.n.) with hMPV (10⁵ PFU per animal). To quantify hMPV load, groups of 5 animals were sacrificed on days 5 and 7 post-infection (the first study) or days 5 and 9 post-infection (the second study) for collection of lungs and noses for viral quantification by plaque assay. A group of normal, age-matched cotton rats was infected with hMPV and sacrificed on days 5, 7, and 9 post-infection for quantification of hMPV by plaque assay.

For the evaluation of dose-dependency of prophylactic and therapeutic efficacy of MPV467, cotton rats immunosuppressed with cyclophosphamide were inoculated i.m. with 0.1, 1, or 10 mg/kg MPV467 one day before or three days after hMPV challenge. Control animals were treated with PBS (mock) one day before hMPV infection. Animals were sacrificed 5 days after hMPV challenge and lungs and noses were collected for viral titration. For analysis of therapeutic efficacy of MPV467 at the time of delayed clearance (day 9), immunosuppressed animals were infected with hMPV, treated i.m. with 10 mg/kg MPV467 on day 3 and sacrificed on day 5 post-infection, or treated with MPV467 on days 3 and 7 and sacrificed on day 9 post-infection. Normal animals were treated with MPV467 3 days post-infection and sacrificed on day 9. Lungs were collected for histopathology and qPCR analysis. Lungs and noses were collected for viral titration.

Viral Titration: Lung and nose homogenates were clarified by centrifugation and diluted in EMEM. Confluent LLC-MK-2 monolayers were infected in duplicates with diluted homogenates in 24 well plates. After one hour incubation at 37° C. in a 5% CO₂ incubator, the wells were overlaid with 0.75% methylcellulose medium. After 7 days of incubation, the overlays were removed, the cells were fixed for one hour and air-dried for immuno-staining. Upon blocking the wells with 1% BSA in PBS, mouse anti-hMPV-N-protein antibody at a 1:1,000 dilution in 1% BSA was added to each well, followed by washes and then incubation with HRP-conjugated Rabbit anti-mouse IgG diluted 1:1,000 in 1% BSA. AEC Chromogen detection solution was added to each well and incubated at room temperature for 2 hours. Visible plaques were counted and virus titers were expressed as plaque forming units per gram of tissue. Viral titers were calculated as geomean±standard error (S.E.M.) for all animals in a group at a given time.

Lung cytokine analysis: Total RNA was extracted from homogenized lung tissue using the RNeasy purification kit (QIAGEN). One micrograms of total RNA was used to prepare cDNA using oligo dT primers and Super Script II RT (Invitrogen). Cotton rat cytokine cDNA were analyzed by qPCR using the primers and conditions previously described (Blanco et al., J Infect Dis 2002; 185:1780-5; Blanco et al. J Interferon Cytokine Res 2004; 24:21-8; Baukhvalova et al., Curr Protoc Cell Biol 2010; Chapter 26:Unit26.6). The signal obtained for each analyzed gene was normalized to the level of β-actin (“housekeeping gene”) expressed in the corresponding organ. Cytokine levels were expressed as the geometric mean±S.E.M. for all animals in a group at a given time. Differences among groups were evaluated by the Student's t-test of summary data.

Histopathology analysis: Lungs were prepared for histopathology analysis as previously described and scored blindly for peribronchiolitis (inflammatory cells around small airways), perivasculitis (inflammatory cells around small blood vessels), alveolitis (inflammatory cells within alveolar spaces), and interstitial pneumonitis (inflammatory cell infiltration and thickening of alveolar walls) (Prince et al., J Gen Virol 2001; 82:2881-8). Each parameter was scored on a 0-4 scale, with 0=0%, 1=5%, 2=25%, 3=75% and 4=100% histological score.

1. An isolated monoclonal antibody or antigen binding fragment thereof, comprising a heavy chain variable (V_{H}) region and a light chain variable region (V_{L}) comprising a heavy chain complementarity determining region (HCDR)1, a HCDR2, and a HCDR3, and a light chain complementarity determining region (LCDR)1, a LCDR2, and a LCDR3 of the V_{H} and V_{L} set forth as:
a) SEQ ID NOs: 41 and 45, respectively (MPV467);
b) SEQ ID NOs: 9 and 13, respectively (MPV414);
c) SEQ ID NOs: 17 and 21, respectively (MPV454);
d) SEQ ID NOs: 25 and 29, respectively (MPV456);
e) SEQ ID NOs: 33 and 37, respectively (MPV464);
f) SEQ ID NOs: 1 and 5, respectively (MPV86);
g) SEQ ID NOs: 49 and 53, respectively (MPV477);
h) SEQ ID NOs: 57 and 61, respectively (MPV478);
i) SEQ ID NOs: 65 and 69, respectively (MPV481);
j) SEQ ID NOs: 73 and 77, respectively (MPV482);
k) SEQ ID NOs: 81 and 85, respectively (MPV483);
l) SEQ ID NOs: 89 and 93, respectively (MPV485);
m) SEQ ID NOs: 97 and 101, respectively (MPV486);
n) SEQ ID NOs: 105 and 109, respectively (MPV487);
o) SEQ ID NOs: 113 and 117, respectively (MPV488);
p) SEQ ID NOs: 121 and 125, respectively (MPV489);
q) SEQ ID NOs: 129 and 133, respectively (MPV491); or
r) SEQ ID NOs: 137 and 141, respectively (MPV503);
wherein the monoclonal antibody specifically binds to human metapneumovirus (hMPV) F protein and neutralizes hMPV.

2. The isolated monoclonal antibody or antigen binding fragment of claim 1, wherein the HCDR1, the HCDR2, the HCDR3, the LCDR1, the LCDR2, and the LCDR3 comprise the amino acids sequences set forth as
a) SEQ ID NOs: 42, 43, 44, 46, 47 (GEN), and 48, respectively;
b) SEQ ID NOs: 10, 11, 12, 14, 15 (GAS), and 16, respectively;
c) SEQ ID NOs: 18, 19, 20, 22, 23 (VAS), and 24, respectively;
d) SEQ ID NOs: 26, 27, 28, 30, 31 (KTS), and 32, respectively;
e) SEQ ID NOs: 34, 35, 36, 38, 39 (QDN), and 40, respectively;
f) SEQ ID NOs: 2, 3, 4, 6, 7, and 8, respectively;
g) SEQ ID NOs: 50, 51, 52, 54, 55 (DNS), and 56, respectively;
h) SEQ ID NOs: 58, 59, 60, 62, 63 (WAS), and 64, respectively;
i) SEQ ID NOs: 66, 67, 68, 70, 71 (GAS), and 72, respectively;
j) SEQ ID NOs: 74, 75, 76, 78, 79 (KDK), and 80, respectively;
k) SEQ ID NOs: 82, 83, 84, 86, 87 (AAS), and 88, respectively;
l) SEQ ID NOs: 90, 91, 92, 94, 95 (QDT), and 96, respectively;
m) SEQ ID NOs: 98, 99, 100, 102, 103 (SDN), and 104, respectively;
n) SEQ ID NOs: 106, 107, 108, 110, 111 (DAS), and 112, respectively;
o) SEQ ID NOs: 114, 115, 116, 118, 119 (WAS), and 120, respectively;
p) SEQ ID NOs: 122, 123, 124, 126, 127, and 128, respectively;
q) SEQ ID NOs: 130, 131, 132, 134, 135 (RGS), and 136, respectively; or
r) SEQ ID NOs: 138, 139, 140, 142, 143 (GAS), and 144, respectively.

3. The isolated monoclonal antibody or antigen binding fragment of claim 1, wherein the V_{H} and the V_{L} comprise the amino acid sequences at least 90% identical to the amino acid sequences set forth as
a) SEQ ID NOs: 41 and 45, respectively;
b) SEQ ID NOs: 9 and 13, respectively;
c) SEQ ID NOs: 17 and 21, respectively;
d) SEQ ID NOs: 25 and 29, respectively;
e) SEQ ID NOs: 33 and 37, respectively;
f) SEQ ID NOs: 1 and 5, respectively;
g) SEQ ID NOs: 49 and 53, respectively;
h) SEQ ID NOs: 57 and 61, respectively;
i) SEQ ID NOs: 65 and 69, respectively;
j) SEQ ID NOs: 73 and 77, respectively;
k) SEQ ID NOs: 81 and 85, respectively;
l) SEQ ID NOs: 89 and 93, respectively;
m) SEQ ID NOs: 97 and 101, respectively;
n) SEQ ID NOs: 105 and 109, respectively;
o) SEQ ID NOs: 113 and 117, respectively;
p) SEQ ID NOs: 121 and 125, respectively;
q) SEQ ID NOs: 129 and 133, respectively; or
r) SEQ ID NOs: 137 and 141, respectively.

4. The isolated monoclonal antibody or antigen binding fragment of claim 1, comprising a human framework region.

5. The isolated monoclonal antibody or antigen binding fragment of claim 1, wherein the V_{H} and the V_{L} comprise the amino acid sequences set forth as:
a) SEQ ID NOs: 41 and 45, respectively;
b) SEQ ID NOs: 9 and 13, respectively;
c) SEQ ID NOs: 17 and 21, respectively;
d) SEQ ID NOs: 25 and 29, respectively;
e) SEQ ID NOs: 33 and 37, respectively;
f) SEQ ID NOs: 1 and 5, respectively;
g) SEQ ID NOs: 49 and 53, respectively;
h) SEQ ID NOs: 57 and 61, respectively;
i) SEQ ID NOs: 65 and 69, respectively;
j) SEQ ID NOs: 73 and 77, respectively;
k) SEQ ID NOs: 81 and 85, respectively;
l) SEQ ID NOs: 89 and 93, respectively;
m) SEQ ID NOs: 97 and 101, respectively;
n) SEQ ID NOs: 105 and 109, respectively;
o) SEQ ID NOs: 113 and 117, respectively;
p) SEQ ID NOs: 121 and 125, respectively;
q) SEQ ID NOs: 129 and 133, respectively; or
r) SEQ ID NOs: 137 and 141, respectively.

6. The isolated monoclonal antibody of claim 1, wherein the antibody comprises a human constant domain.

7. The isolated monoclonal antibody of claim 1, wherein the antibody is a human antibody.

8. The isolated monoclonal antibody of claim 1, wherein the antibody is an IgG.

9. The isolated monoclonal antibody of claim 1, comprising a recombinant constant domain comprising a modification that increases the half-life of the antibody.

10. The isolated monoclonal antibody of claim 9, wherein the modification increases binding to the neonatal Fc receptor.

11. The isolated monoclonal antibody or antigen binding fragment of claim 1, wherein the antibody neutralizes group A and group B hMPV.

12. The isolated monoclonal antibody or antigen binding fragment of claim 1, wherein the antibody binds pre-fusion F protein with a higher affinity than post-fusion F protein.

13. The isolated monoclonal antigen binding fragment of claim 1.

14. The isolated monoclonal antigen binding fragment of claim 13, wherein the antigen binding fragment is a Fv, Fab, F(ab′)₂, scFV or a scFV₂ fragment.

15. The isolated monoclonal antibody of claim 1, or the antigen binding fragment thereof, conjugated to an effector molecule or a detectable marker.

16. A bispecific antibody comprising the monoclonal antibody or the antigen binding fragment of claim 1.

17. An isolated nucleic acid molecule encoding the monoclonal antibody or the antigen binding fragment of claim 1, or, or a V_{H} or V_{L} of the monoclonal antibody or antigen binding fragment.

18. The nucleic acid molecule of claim 17, wherein the nucleic acid molecule is a cDNA sequence.

19. The nucleic acid molecule of claim 17, operably linked to a promoter.

20. A vector comprising the nucleic acid molecule of claim 17.

21. A host cell comprising the nucleic acid molecule of claim 17, or vector comprising the nucleic acid molecule.

22. A pharmaceutical composition for use in inhibiting an hMPV infection, comprising an effective amount of the monoclonal antibody or the antigen binding fragment of claim 1, nucleic acid molecule encoding the monoclonal antibody or the antigen binding fragment, or vector comprising the nucleic acid molecule; and
a pharmaceutically acceptable carrier.

23. A method of producing a monoclonal antibody or antigen binding fragment that specifically binds to hMPV F protein, comprising:
expressing one or more nucleic acid molecules encoding the monoclonal antibody or the antigen binding fragment of claim 1 or the or a bispecific antibody comprising the monoclonal antibody or the antigen binding fragment of in a host cell; and
purifying the antibody or antigen binding fragment.

24. A method of detecting the presence of hMPV in a biological sample from a human subject, comprising:
contacting the biological sample with an effective amount of the monoclonal antibody or antigen binding fragment of claim 1, or a bispecific antibody comprising the monoclonal antibody or antigen binding fragment, under conditions sufficient to form an immune complex; and
detecting the presence of the immune complex in the biological sample, wherein the presence of the immune complex in the biological sample indicates the presence of the hMPV in the sample.

25. The method of claim 26, wherein detecting the detecting the presence of the immune complex in the biological sample indicates that the subject has an hMVP infection.

26. A method of inhibiting an hMPV infection in a subject, comprising administering an effective amount of the monoclonal antibody or antigen binding fragment of claim 1 a bispecific antibody comprising the antibody or antigen binding fragment, a nucleic acid molecule encoding the monoclonal antibody, antigen binding fragment or bispecific antibody, a vector comprising the nucleic acid molecule, to the subject, thereby inhibiting the hMPV infection in the subject.

27. (canceled)


SEQUENCE LISTING (148 sequences)

SEQ ID NO: 1 - protein, 130 aa, Homo sapiens
QVQLVQSGAEVKKPGSSVKVSCKASGDTFNDVALSWVRQAPGQGLEWLGGIIPFFGTANYAQRFQDRVTITADASTSTAYLELGGLRSEDTAVYYCARGWSCRNITCFMMGYYFYIMDVWGQGTLVTVSS

SEQ ID NO: 2 - protein, 8 aa, Homo sapiens
GDTFNDVA

SEQ ID NO: 3 - protein, 8 aa, Homo sapiens
IIPFFGTA

SEQ ID NO: 4 - protein, 23 aa, Homo sapiens
ARGWSCRNITCFMMGYYFYIMDV

SEQ ID NO: 5 - protein, 97 aa, Homo sapiens
LSLSPGERATLSCRASQSVGNFLAWYQQKPGQTPRLLIQDASNRATGIPARFSGTGSGTDFTLTISSLEPEDFAVYYCQQRSNWPPTFGPGTKVDIK

SEQ ID NO: 6 - protein, 6 aa, Homo sapiens
QSVGNF

SEQ ID NO: 7 - skipped

SEQ ID NO: 8 - protein, 9 aa, Homo sapiens
QQRSNWPPT

SEQ ID NO: 9 - protein, 125 aa, Homo sapiens
QVQLVQSGPEVKKPGSSVMVSCKTSGGNFNNFAISWVRQAPGQGLEWMGGIVPIFGTANYAQKSQDRVTITADPSSSTAYLQLSSLTSDDTAIYYCARDLGRSSNYYYYSVFESWGQGTLVTVSS

SEQ ID NO: 10 - protein, 8 aa, Homo sapiens
GGNFNNFA

SEQ ID NO: 11 - protein, 8 aa, Homo sapiens
IVPIFGTA

SEQ ID NO: 12 - protein, 18 aa, Homo sapiens
ARDLGRSSNYYYYSVFES

SEQ ID NO: 13 - protein, 108 aa, Homo sapiens
DIVMTQSPATLSLSPGERATLSCRASQSVGDKLAWYQQKPGQAPRLLIYGASTRATDIPARFSGSGSGTEFTLTVSSLQPEDFAVYYCQQYENWPPITFGQGTRLEIK

SEQ ID NO: 14 - protein, 6 aa, Homo sapiens
QSVGDK

SEQ ID NO: 15 - skipped

SEQ ID NO: 16 - protein, 10 aa, Homo sapiens
QQYENWPPIT

SEQ ID NO: 17 - protein, 122 aa, Homo sapiens
EVQLVESGGGLVKPGGSLRLSCSTSGFVFSSYAMHWVRQAPGKGLEHVSAISSTGDNTHYADSVKGRFTISRDNSRGRLYLQMTSVRPEDAALYYCVKDLYSGRFYYFLDDWGQGTRIIVSS

SEQ ID NO: 18 - protein, 8 aa, Homo sapiens
GFVFSSYA

SEQ ID NO: 19 - protein, 8 aa, Homo sapiens
ISSTGDNT

SEQ ID NO: 20 - protein, 15 aa, Homo sapiens
VKDLYSGRFYYFLDD

SEQ ID NO: 21 - protein, 108 aa, Homo sapiens
DIQVTQSPSSLSASVGDRVTISCRASQNISNYLNWFQQRPGKAPKLLIYVASTLQSGAPSRFSGSGSGTDFTLTITDLQPEDFATYYCQQSYNTPPITFGQGTRLEIK

SEQ ID NO: 22 - protein, 6 aa, Homo sapiens
QNISNY

SEQ ID NO: 23 - skipped

SEQ ID NO: 24 - protein, 10 aa, Homo sapiens
QQSYNTPPIT

SEQ ID NO: 25 - protein, 128 aa, Homo sapiens
QVQLQESGPGVVKPSGTLSLTCGVSGGSISSSYWWSWVRQPPGKGLEWIGEIYHSGSTNYNPSLKRRVTISVDKSKNQFSLKVTSVTAADTAVYYCARGPIQYYDILTEERVGYCDYWGQGTLVTVSS

SEQ ID NO: 26 - protein, 9 aa, Homo sapiens
GGSISSSYW

SEQ ID NO: 27 - protein, 7 aa, Homo sapiens
IYHSGST

SEQ ID NO: 28 - protein, 21 aa, Homo sapiens
ARGPIQYYDILTEERVGYCDY

SEQ ID NO: 29 - protein, 98 aa, Homo sapiens
TLSASVGDRVTITCRASQSISSWLAWFQQKPGKAPKLLIYKTSSLESGVPSRFSGSGSGTEFTLTISSLQPDDFATYYCQEYNSYSRTFGQGTKVEIK

SEQ ID NO: 30 - protein, 6 aa, Homo sapiens
QSISSW

SEQ ID NO: 31 - skipped

SEQ ID NO: 32 - protein, 9 aa, Homo sapiens
QEYNSYSRT

SEQ ID NO: 33 - protein, 124 aa, Homo sapiens
QVQLQESGPGLVKPSQTLSLTCTVSGGSVRGGGDYWSWIRRPPGKGLEWIGHIYNSGTTFYNPSLKSRVTISIDTSKNQFSLKLRSVTAADTAVYYCGRVGSRATGLPDWIDPWGQGTLVTVSS

SEQ ID NO: 34 - protein, 10 aa, Homo sapiens
GGSVRGGGDY

SEQ ID NO: 35 - protein, 7 aa, Homo sapiens
IYNSGTT

SEQ ID NO: 36 - protein, 16 aa, Homo sapiens
GRVGSRATGLPDWIDP

SEQ ID NO: 37 - protein, 106 aa, Homo sapiens
SYELTQPPSVSVSPGQTATITCSGTNLGAKYSCWYQQKPGQSPVLVIFQDNKRASGIPARFSASTSGDTATLTISGTQVMDEADYFCQAWDSRTAVFGGGTTLTVL

SEQ ID NO: 38 - protein, 6 aa, Homo sapiens
NLGAKY

SEQ ID NO: 39 - skipped

SEQ ID NO: 40 - protein, 9 aa, Homo sapiens
QAWDSRTAV

SEQ ID NO: 41 - protein, 124 aa, Homo sapiens
EVQLVESGGGVVRPGTSLRVSCAAFDFNFRDYGMHWVRQAPGKGLEWVAGIWYDGSNKDYADSVKGRFTISRDNSQNTLYLQMNSLRVEDTAVYYCARDPRTHREGALSHFDSWGQGTLVTVSS

SEQ ID NO: 42 - protein, 8 aa, Homo sapiens
DFNFRDYG

SEQ ID NO: 43 - protein, 8 aa, Homo sapiens
IWYDGSNK

SEQ ID NO: 44 - protein, 17 aa, Homo sapiens
ARDPRTHREGALSHFDS

SEQ ID NO: 45 - protein, 108 aa, Homo sapiens
PNELTQDPAVSVALGQTVRITCQGDSLRNYFAGWYQQKPGQAPLLVLYGENIRPSGIPDRFSGSSSGNTVSLTITGAQAEDEADYYCNSRDNSGNHWVFGGGTRLTVL

SEQ ID NO: 46 - protein, 6 aa, Homo sapiens
SLRNYF

SEQ ID NO: 47 - skipped

SEQ ID NO: 48 - protein, 11 aa, Homo sapiens
NSRDNSGNHWV

SEQ ID NO: 49 - protein, 128 aa, Homo sapiens
EVQLVQSGAEVKKPGASVEVSCKTSGYTFTDYYLHWVRQAPGQGLEWMGWINPRSGGTKYGQKFQGRVTMTRDTSISTAYMDLRGLRSDDTAVYYCARAPLLTVYAVAHRSGENRFDPWGQGTVTVSS

SEQ ID NO: 50 - protein, 8 aa, Homo sapiens
GYTFTDYY

SEQ ID NO: 51 - protein, 8 aa, Homo sapiens
INPRSGGT

SEQ ID NO: 52 - protein, 22 aa, Homo sapiens
ARAPLLTVYAVAHRSGENRFDP

SEQ ID NO: 53 - protein, 106 aa, Homo sapiens
QPPSLSGAPGQTVTISCTGSGSNIGAGYDVNWYQCLPGTAPKLLMFDNSNRPSGVPDRFSGSRSGASASLAITGLQAEDEADYYCQSYDSGLSGWVFGGGTKLTVL

SEQ ID NO: 54 - protein, 9 aa, Homo sapiens
GSNIGAGYD

SEQ ID NO: 55 - skipped

SEQ ID NO: 56 - protein, 11 aa, Homo sapiens
QSYDSGLSGWV

SEQ ID NO: 57 - protein, 128 aa, Homo sapiens
EVQLVQSGAEVKKPGASVKVSCNASGYTFTGYYIHWVRQAPGQGLEWMGWINPRSGGTNYAQKFQGRVTLTRDTSITTAYMDVTRLRPDDTAVYYCATTREGIVLMPRGRGDDALDTWGQGTTVIVSS

SEQ ID NO: 58 - protein, 8 aa, Homo sapiens
GYTFTGYY

SEQ ID NO: 59 - protein, 8 aa, Homo sapiens
INPRSGGT

SEQ ID NO: 60 - protein, 21 aa, Homo sapiens
ATTREGIVLMPRGRGDDALDT

SEQ ID NO: 61 - protein, 113 aa, Homo sapiens
DIVMTQSPVSLTVSLGERATINCKSSQSVLSTSNNENYLAWYQQKPGQPPNLLIYWASTRESGVPDRFSGSGSGTDFTLTISSLQAEDVAVYYCQQYFATPITFGPGTKVDVK

SEQ ID NO: 62 - protein, 12 aa, Homo sapiens
QSVLSTSNNENY

SEQ ID NO: 63 - skipped

SEQ ID NO: 64 - protein, 9 aa, Homo sapiens
QQYFATPIT

SEQ ID NO: 65 - protein, 124 aa, Homo sapiens
EVQLVQSGGGLVKPGGSLRLSCAASGLPFNNAWMNWVRQAPGKGLEWVGRIKSKIDGGTTEYAAPVKGRFTISRDDSENTLYLQMNSLRTEDTAVYYCTTEYYALLSGNYSDCWGQGTLVTVSS

SEQ ID NO: 66 - protein, 8 aa, Homo sapiens
GLPFNNAW

SEQ ID NO: 67 - protein, 10 aa, Homo sapiens
IKSKIDGGTT

SEQ ID NO: 68 - protein, 15 aa, Homo sapiens
TTEYYALLSGNYSDC

SEQ ID NO: 69 - protein, 109 aa, Homo sapiens
DIVMTQSPFSLSLSPGERATLSCRASQSIKRAYLGWYQQKPGQAPRLLIYGASNRATGIPDRFSGSGSGTDFTLTISRLEPEDFAVYYCQQYGDSPGGSFGQGTKLEIR

SEQ ID NO: 70 - protein, 7 aa, Homo sapiens
QSIKRAY

SEQ ID NO: 71 - skipped

SEQ ID NO: 72 - protein, 10 aa, Homo sapiens
QQYGDSPGGS

SEQ ID NO: 73 - protein, 122 aa, Homo sapiens
EVQLVQSGGGVVQPGRSLRLSCAASGFTFSSFAMHWVRQAPGKGLEWVALISDDGNNKYHADSVKGRFTISRDSSKNTLYLQMNSLRGEDTAVYYCARDGSVVGAAAHCDHWGQGTLVTVSS

SEQ ID NO: 74 - protein, 8 aa, Homo sapiens
GFTFSSFA

SEQ ID NO: 75 - protein, 8 aa, Homo sapiens
ISDDGNNK

SEQ ID NO: 76 - protein, 15 aa, Homo sapiens
ARDGSVVGAAAHCDH

SEQ ID NO: 77 - protein, 107 aa, Homo sapiens
SYELTQPPSVSVSPGQTARITCSGDALPNQCAYWYQQKPGLAPVLLIYKDKERPSGIPERFSGSISGTTVTLTISGVQAEDEADYYCQSSDSSSTVVFGGGTKLTVL

SEQ ID NO: 78 - protein, 6 aa, Homo sapiens
ALPNQC

SEQ ID NO: 79 - skipped

SEQ ID NO: 80 - protein, 10 aa, Homo sapiens
QSSDSSSTVV

SEQ ID NO: 81 - protein, 130 aa, Homo sapiens
EVQLVQSGAELKKPGSSVKVSCKASGDTFSNYAISWVRQAPGQGLEWMGGIIPIYNTANYAQKFQGRVTLTADESTSTAYMELSSLRSEDTAVYYCARDVRNNWSVLRGARYYYYGMDVWGQGTTVTVSS

SEQ ID NO: 82 - protein, 8 aa, Homo sapiens
GDTFSNYA

SEQ ID NO: 83 - protein, 8 aa, Homo sapiens
IIPIYNTA

SEQ ID NO: 84 - protein, 23 aa, Homo sapiens
ARDVRNNWSVLRGARYYYYGMDV

SEQ ID NO: 85 - protein, 106 aa, Homo sapiens
DIVMTQTPFSLSASVGDRVTVTCRASQDISNSLAWFQQKPGKAPKSLIYAASSLQSGVPTRFSGGGSGTDFTLTISSLQPEDFATFYCLQYDSFPPTFGGGTKVEI

SEQ ID NO: 86 - protein, 6 aa, Homo sapiens
QDISNS

SEQ ID NO: 87 - skipped

SEQ ID NO: 88 - protein, 9 aa, Homo sapiens
LQYDSFPPT

SEQ ID NO: 89 - protein, 125 aa, Homo sapiens
EVQLQEPGPGLVKPSETLSLTCTVSGGSISSTNSFWGWVRQPPGKGLEWIGSIYYSGTTYYNSSLKSRVTISVDTSKNQFSLRLSSVTAADTAVYYCARQGLTSSWYDGSGLDVWGRGTKVTVSS

SEQ ID NO: 90 - protein, 10 aa, Homo sapiens
GGSISSTNSF

SEQ ID NO: 91 - protein, 7 aa, Homo sapiens
IYYSGTT

SEQ ID NO: 92 - protein, 17 aa, Homo sapiens
ARQGLTSSWYDGSGLDV

SEQ ID NO: 93 - protein, 106 aa, Homo sapiens
SYELTQPPSVSVSPGQTASITCSGDKLGNKYACWYQQRPGQSPVLVIYQDTKRPSGIPERFSGSNSGNTATLTISGTQAMDEADYYCQAWDSNTVVFGGGTKLTVL

SEQ ID NO: 94 - protein, 6 aa, Homo sapiens
KLGNKY

SEQ ID NO: 95 - skipped

SEQ ID NO: 96 - protein, 9 aa, Homo sapiens
QAWDSNTVV

SEQ ID NO: 97 - protein, 126 aa, Homo sapiens
EVQLVESGGHLVQSGGSLRLSCAASGFTFSNYILNWVRQAPGKGLEWISYITGGSSAIYYADSVKGRFTISRDDAKNSLYLQMNNLRAEDTAVYYCARSHGYDSSGYYYYFAMDVWGQGTTVTVSS

SEQ ID NO: 98 - protein, 8 aa, Homo sapiens
GFTFSNYI

SEQ ID NO: 99 - protein, 8 aa, Homo sapiens
ITGGSSAI

SEQ ID NO: 100 - protein, 19 aa, Homo sapiens
ARSHGYDSSGYYYYFAMDV

SEQ ID NO: 101 - protein, 109 aa, Homo sapiens
QSVLTQPPSASGTPGQRVTISCSGSNSNIESNTVNWYQQIPGTAPKLLIYSDNRRPSGVPDRFSGSKSGTSASLAISGLQSEDEADYYCAAWDDSLIGYVFGTGTKVTV

SEQ ID NO: 102 - protein, 8 aa, Homo sapiens
NSNIESNT

SEQ ID NO: 103 - skipped

SEQ ID NO: 104 - protein, 11 aa, Homo sapiens
AAWDDSLIGYV

SEQ ID NO: 105 - protein, 124 aa, Homo sapiens
EVQLLESGGGLVQPGRSLRLSCTASGFTFDDYAMHWVRQLPGKGLEWVSGISWNSDNIGYADSVKGRFTISRDNGKNSLYLQMNSLRAEDTAFYYCAKDVRIDYDLLIGHIDYWGPGTLVTVSS

SEQ ID NO: 106 - protein, 8 aa, Homo sapiens
GFTFDDYA

SEQ ID NO: 107 - protein, 8 aa, Homo sapiens
ISWNSDNI

SEQ ID NO: 108 - protein, 17 aa, Homo sapiens
AKDVRIDYDLLIGHIDY

SEQ ID NO: 109 - protein, 101 aa, Homo sapiens
TQSPVTLSLSPGERATLSCRASQSFGGYCAWYQQKPGQSPRLLIYDASNRATGVPARFSGSGSGTDFTLTISSLEPEDSAVYFCQQRGNWPIFGQGTRLEI

SEQ ID NO: 110 - protein, 6 aa, Homo sapiens
QSFGGY

SEQ ID NO: 111 - skipped

SEQ ID NO: 112 - protein, 8 aa, Homo sapiens
QQRGNWPI

SEQ ID NO: 113 - protein, 130 aa, Homo sapiens
MPVVHEVQLVQSGFEVKKPGASVKVSCKASGYNFNNYGISWVRQAPGQGLEWMGWISAYTGNTNEAQKFQGRVSMTTDTSTSTAYMEVRSLRSDDTAVYYCARDIGSSMFYSYFYGMDVWGQGTTVIVSS

SEQ ID NO: 114 - protein, 8 aa, Homo sapiens
GYNFNNYG

SEQ ID NO: 115 - protein, 8 aa, Homo sapiens
ISAYTGNT

SEQ ID NO: 116 - protein, 18 aa, Homo sapiens
ARDIGSSMFYSYFYGMDV

SEQ ID NO: 117 - protein, 104 aa, Homo sapiens
SLSVSLGERATINCKSSESVLYNSNNENYLDWYQQKPGQPPKLLIYWASTRASGVPDRFSGSASGTDFTLTISSLQAEDVAVYYCQQYYSTPRTFGQGTKVEIK

SEQ ID NO: 118 - protein, 12 aa, Homo sapiens
ESVLYNSNNENY

SEQ ID NO: 119 - skipped

SEQ ID NO: 120 - protein, 9 aa, Homo sapiens
QQYYSTPRT

SEQ ID NO: 121 - protein, 121 aa, Homo sapiens
EVQLLESGGGLVQPGGSLRLSCAASKFTFNNYEMNWLRQAPGKGLEWVSSISSSGDTIHNADSVKGRFIISRDNAKNSLHLQMNGLRAEDTAVYYCARAGSGWAYDAFDIWGLGTMVTVSS

SEQ ID NO: 122 - protein, 8 aa, Homo sapiens
KFTFNNYE

SEQ ID NO: 123 - protein, 8 aa, Homo sapiens
ISSSGDTI

SEQ ID NO: 124 - protein, 14 aa, Homo sapiens
ARAGSGWAYDAFDI

SEQ ID NO: 125 - protein, 97 aa, Homo sapiens
LSASVGDRVTITCRASQSISSFLNWYQQKPGKAPKLLMYAASSLPVGVPSRFSGSGSGPEYTLTISNLQPEDFATYYCQQGYSSPPTFGGGTRLEVK

SEQ ID NO: 126 - protein, 6 aa, Homo sapiens
QSISSF

SEQ ID NO: 127 - skipped

SEQ ID NO: 128 - protein, 9 aa, Homo sapiens
QQGYSSPPT

SEQ ID NO: 129 - protein, 124 aa, Homo sapiens
QVQLQESGPGLVKSSQTLSLTCTVSGGSISSVTSHWTWIRQHPGKGLEWIGYIFSSGTTYYSPSLRSRLTMSVDTSKNQFSLQLSSVTAADTAMYYCARGIYCGDSCYKGTDYWGRGTLVTVSS

SEQ ID NO: 130 - protein, 6 aa, Homo sapiens
GGSISS

SEQ ID NO: 131 - protein, 7 aa, Homo sapiens
IFSSGTT

SEQ ID NO: 132 - protein, 16 aa, Homo sapiens
ARGIYCGDSCYKGTDY

SEQ ID NO: 133 - protein, 112 aa, Homo sapiens
IRLMTQTPVTLPVTPGEPASISCRSSQSLLHSNGYNYLDWYLQKPGQSPQLLIYRGSTRASGVPDRFSGSGSGTEFTLKISRVEAEDVGVYYCMQGLQTPYTFGQGTKLESR

SEQ ID NO: 134 - protein, 11 aa, Homo sapiens
QSLLHSNGYNY

SEQ ID NO: 135 - skipped

SEQ ID NO: 136 - protein, 9 aa, Homo sapiens
MQGLQTPYT

SEQ ID NO: 137 - protein, 126 aa, Homo sapiens
EVQLVQSGAEVKKPGSSVKVSCKASGGTFSSYTFSWVRQAPGQGLEWMGGIIPIFGTANYAQKFQDRVTITADESTSTAYMELSSLRSEDTAVYYCARGRVVVIASHYYYYDMDVWGQGTTVTVSS

SEQ ID NO: 138 - protein, 8 aa, Homo sapiens
GGTFSSYT

SEQ ID NO: 139 - protein, 8 aa, Homo sapiens
IIPIFGTA

SEQ ID NO: 140 - protein, 19 aa, Homo sapiens
ARGRVVVIASHYYYYDMDV

SEQ ID NO: 141 - protein, 100 aa, Homo sapiens
VSLSVSPGERATLSCRASQSVSSNLAWYQQKPGQAPRLLIYGASTRATGIPARFSGSGSGTEFTLTISSLQSEDFAVYYCQQYDNWPPLTFGGGTKVEIK

SEQ ID NO: 142 - protein, 6 aa, Homo sapiens
QSVSSN

SEQ ID NO: 143 - skipped

SEQ ID NO: 144 - protein, 10 aa, Homo sapiens
QQYDNWPPLT

SEQ ID NO: 145 - protein, 122 aa, Homo sapiens
EVQLLESGGGLVQPGGSRRLSCAASGFTVSSSYMSWVRQTPGKGLEWISVFYSGGTTYYADAVKGRFSISMDTSKNTLHLQMNSLRVEDTAIYYCARVLSRASGMPDAFDIWGPGTMVTVSS

SEQ ID NO: 146 - protein, 108 aa, Homo sapiens
ELALIQPASVSVSPGQTASITCSGDKLGDKYASWYQQKPGQSPVLVIYQDSERPSGIPERFSGSNSGNTATLTISGTQAMDEADYYCQAWDSSTAVFGGGTTLTVLGQ

SEQ ID NO: 147 - protein, 124 aa, Homo sapiens
EVQLVESGGGLVKPGGSLRLSCAASGFTFSSYSMNWVRQAPGKGLEWVSSISASSSYSDYADSAKGRFTISRDNAKTSLFLQMNSLRAEDTAIYFCARARATGYSSITPYFDIWGQGTLVTVSS

SEQ ID NO: 148 - protein, 110 aa, Homo sapiens
QSVVTQPPSVSGAPGQRVTISCTGSSSNIGAGYDVHWYQQLPGTAPKLLIYDNNNRPSGVPDRFSASKSGTSASLAITGLQAEDEADYYCQSYDRSLSGVFGTGTKVTVL